(19) United States
(12) Patent Application Publication
(10) Pub. No.: US 2013/0096010 A1
(43) Pub. Date: Apr. 18, 2013

(54) HtSNPs FOR DETERMINING A GENOTYPE OF CYTOCHROME P450 1A2, 2A6 AND 2D6, PXR AND UDP-GLUCURONOSYLTRANSFERASE 1A GENE AND MULTIPLEX GENOTYPING METHODS USING THEREOF

(71) Applicants: Jae-Gook SHIN, Busan (KR); Yin-jin JANG, Busan (KR); Sang-seop LEE, Busan (KR); Hye-eun JEONG, Busan (KR); In-june CHA, Busan (KR); Woo-young KIM, Busan (KR); Sung-su YEA, Busan (KR); Eun-young KIM, Busan (KR); Eun-young CHA, Busan (KR); Ji-hong SHON, Busan (KR); Eun-jeong CHOI, Busan (KR); Kang-mi KIM, Busan (KR); Hyun-ju JUNG, Busan (KR)

(72) Inventors: Jae-Gook SHIN, Busan (KR); Yin-jin JANG, Busan (KR); Sang-seop LEE, Busan (KR); Hye-eun JEONG, Busan (KR); In-june CHA, Busan (KR); Woo-young KIM, Busan (KR); Sung-su YEA, Busan (KR); Eun-young KIM, Busan (KR); Eun-young CHA, Busan (KR); Ji-hong SHON, Busan (KR); Eun-jeong CHOI, Busan (KR); Kang-mi KIM, Busan (KR); Hyun-ju JUNG, Busan (KR)

(73) Assignee: INJE UNIVERSITY INDUSTRY ACADEMIC COOPERATION FOUNDATION, Kimhae City (KR)

(21) Appl. No.: 13/549,873

(22) Filed: Jul. 16, 2012

Related U.S. Application Data
(62) Division of application No. 12/440,634, filed on Mar. 10, 2009, now abandoned.

(30) Foreign Application Priority Data
Sep. 11, 2006 (KR) 10-2006-0087179
May 30, 2007 (KR) 10-2007-0052764
Jun. 18, 2007 (KR) 10-2007-0059244
Jun. 18, 2007 (KR) 10-2007-0059245
Jun. 18, 2007 (KR) 10-2007-0059247
Jun. 18, 2007 (KR) 10-2007-0059248

(51) Int. Cl. C12Q 1/68

(52) U.S. Cl. CPC C12Q 1/6869
USPC 506/2; 435/6.11

(57) ABSTRACT

The present invention relates to htSNPs for determining a genotype of cytochrome P450 1A2 (CYP1A2), 2A6 (CYP2A6) and 2D6 (CYP2D6), PXR and UDP-glucuronosyltransferase 1a (UGT1A) genes and a gene chip using the same, and more particularly, to a selection method of htSNPs for determining a haplotype of human CYP1A2, CYP2A6, CYP2D6, PXR and UGT1A genes, a method of determining a genotype of the genes by using the htSNPs and a gene chip therefor.

### CROSS-REFERENCES TO RELATED APPLICATION

This application is a Divisional application of U.S. patent application Ser. No. 12/440,634 filed Mar. 10, 2009, which is a National Stage application of PCT/KR2007/003102 filed on Jun. 26, 2012, which claims priority to Korean Patent Application Nos. KR10-2006-0087179 filed on 2006 Sep. 11, KR10-2007-0052764 filed on 2007 May 30, KR10-2007-0059244 filed on 2007 Jun. 18, KR10-2007-0059245 filed on 2007 Jun. 18, KR10-2007-0059248 2007 Jun. 18, and KR10-2007-0059247 filed 2007 Jun. 18, the contents of which are incorporated herein by reference.

### FIELD OF THE INVENTION

Apparatuses and methods consistent with the present invention relate to HTSNPs for determining a genotype of cytochrome P450 1A2, 2A6 and 2D6, PXR and UDP-glucuronosyltransferase 1a genes and a gene chip, and more particularly, to a selection method of HTSNPs for determining haplotypes of human CYP1A2, CYP2A6, CYP2D6, NR1I2 (=PXR) and UGT1A genes, a method of determining a genotype of the genes and a gene chip therefor.

### BACKGROUND ART

Individuals react to toxicity and effect of drugs differently due to genetic diversity. Thus, it is essential to consider the effects of pharmaceutically-important protein with respect to the genetic diversity in an initial development stage of drugs, since it can lower potential failure in drug development. Haplotype is one of factors to determine the genetic diversity between individuals. The haplotype refers to a combination of polymorphism of each genetic sequence in a single study population. The haplotype provides more accurate and reliable information about the genetic diversity than individual polymorphism.

Human cytochrome P450 is a part of hemoproteins which facilitate oxidation of exogenous chemical substances such as drugs, carcinogen and toxin and internal substrates such as steroid, fatty acid and vitamine (Nelson et al., Pharmacogenetics 6:1-42, 1996). Various subfamilies of cytochrome P450 are found in the liver, kidney, intestines and lung.

Human cytochrome P450 1A2 (hereinafter, to be called CYP1A2) gene is a drug-metabolizing enzyme included in CYP1 genes, together with CYP1A1 and CYP1B1. CYP1A2 is mainly produced in the liver, and accounts for 15% of the total amount of cytochrome enzymes. CYP1A2 is involved in metabolism of medically-important drugs like caffeine, clozapine, imiparamine and propranolol. Also, CYP1A2 catalyzes internal synthetic substance such as 17β-estradiol, uroporphyrinogen III and carcinogen bioactivation such as polycyclic aromatic hydrocarbon epoxidation and aromatic/heterocyclic amine N-hydroxylation (Brosen K., Clinical Parmacokinetic, 1995, 29 (suppl1): 20-25; Josephy P D., Environ. Mol. Mutagen, 2001, 38:12-18). Human cytochrome P450 2A6 (hereinafter, to be called CYP2A6) gene is located on chromosome 19, and CYP2A7, pseudogen, which has very similar genetic sequences is placed on a CYP2A6 gene. CYP2A6 enzyme is a major enzyme which converts nicotine into cotinine and is involved in approximately 80% of metabolism of nicotine. The CYP2A6 enzyme converts tegafur, anticancer drug, into 5-fluorouracil (5-FU), the active drug in vivo. The enzyme is mainly produced by liver, and expressed in a small quantity in organs such as the lung, large intestine, breast, kidney and uterus (Drus Metab Dispos., 29 (2): 91-5, 2001; Adv Drug Deliv Rev., 18; 54 (10):1245-56, 2002).

Human cytochrome P450 2D6 (hereinafter, to be called CYP2D6) gene is located in chromosome 22, and CYP2D7 and CYP2D8 genes, pseudogenes, are placed in one side of the CYP2D6 gene. Enzymes which are coded by the gene are known to be responsible for metabolism of 100 or more, clinically-important drugs including psychoactive drugs, cardiovascular drugs, morphine drugs, etc.

The enzymes which are coded by the CYP2D6 gene are mainly produced by the liver. Even though the enzymes account for approximately 2% of the total amount of cytochrome P450 enzymes, they are major enzymes involved in 30% of drug metabolism.

The activity of the enzymes is diverse in individuals, and the enzymes are classified into PM (poor metabolizers) IM (intermediate metabolizers) EM (extensive metabolizers) and UM (ultrarapid metabolizers) depending on the degree of activity. Partly, the genetic polymorphism of the genes causes diverse activities of the enzymes. It is known that a CYP1A2 gene demonstrates genetic polymorphism. Twenty-four variants or more are found in promoters, exons and introns of the CYP1A2 gene up to now. As of June, 2007, there are 36 haplotypes, combination of genetic variants, (http://www.cypalleles.ki.se/cyp1a2.htm), 50 genotypes of CYP2A6 (http://www.cypalleles.ki.se.cyp2a6.htm) and approximately 80 genetic polymorphisms of CYP2D6 gene (www.immi.ki.se.cypalleles/cyp2d6.htm), which are significantly different between species. As various types of gene variants and haplotypes have been reported, it is essential to determine an accurate haplotype through the minimum single nucleotide polymorphism (SNP) to thereby enhance time and cost efficiency.

Until various kinds of drugs are taken and discharged from the human body, metabolism and transport occur. Cytochrome P450 (CYP) and drug-transport proteins are involved in the metabolism and transport. Studies on CYP enzymes involved in the metabolism of drugs have been actively conducted. Currently, 15 CYP enzymes, particularly CYP2D6, CYP2C9, CYP3A4, CYP2B6, MDR1 and CYP2C19, have been reported to have genetic polymorphism. The genetic polymorphism serves as a major factor which has influence on clinical effect, treatment effect and side-effect of substrate drug of the enzyme. Some genetic variants cause enzyme deposition cannot metabolize drugs at all. Other genetic variants partly decrease in enzyme activity. For example, enzymes such as CYP2D6 and CYP 2C9 vary in phenotypes depending on the genetic variants and have relatively high similarities between genotypes and phenotypes. Meanwhile, it is difficult to predict phenotypes of CYP3A4, CYP2B6 and MDR1 genes depending on the presence and absence of functional genetic variants.

In terms of humans, CYP3A4 which metabolizes 50% of all taken drugs demonstrates significant differences in activity between individuals. CYP2B6 is known to represent a maximum of 270 times of differences between individuals. Despite such individual differences, activity differences between individuals are difficult to be predicted directly from genotypes, since protein expression of drug-metabolizing enzymes or drug-transport proteins which have low relevance between genotypes and phenotypes varies greatly depending on external factors. As for those genes, expression adjustment of proteins, rather than presence and absence of genetic variants, can be more important factors causing individual differences in metabolic activity. As the expression of the enzymes is induced, enzymes themselves are produced in large amounts to boost activity. The mechanism of expression induction is established by coupling external materials including drug receptors with a promoter of a target gene. A classic example of the drug receptors is pregnane X receptor (PXR), which is known to be expressed in an NR1I2 gene.

The expression amount of PXR reportedly varies depending on individuals. Interestingly, the expression amount of the receptor has high relevance to the expression amount of drug-metabolizing enzymes such as CYP3A4 and CYP2B6 (Current Drug Metabolism, 2005, 6:369-383). Accordingly, the differences of the expression amount of the drug-metabolizing enzymes between individuals result from the difference of the expression amount of the PXR gene or the difference of activity rather than from variant proteins.

In this regard, studies on genetic polymorphism of individual PXR genes or on expression differences of the PXR gene have recently drawn attention. It has been reported that some genetic variants cause individual differences to drug reaction such as increase in CYP3A4 activity by erythromycin breath test or rifampin in the body even though amino acid sequence is not changed (Pharmacogenetics, 2001, 11:555 572). Such PXR variants cause activity change due to expression change of the target gene. Thus, the PXR variants may cause difference of activity of drugs or biomolecules in vivo, and contribute greatly to individual differences of drug interaction by drug, a coupler of a PXR gene.

UDP-glucuronosyltransferase (UGT) is an enzyme which catalyzes glucuronic acid to couple with endogenous and exogenous materials in the body. The UDP-glucuronosyltransferase generates glucuronic acid coupler of materials having toxicity such as phenol, alcohol, amine and fatty acid compound, and converts such materials into hydrophilic materials to be excreted from the body via bile or urine (Parkinson A, Toxicol Pathol., 24:48-57, 1996).

The UGT is reportedly present mainly in endoplasmic reticulum or nuclear membrane of interstitial cells, and expressed in other tissues such as the kidney and skin. The UGT enzyme can be largely classified into UGT1 and UGT2 subfamilies based on similarities between primary amino acid sequences. The human UGT1A family has nine isomers (UGT1A1, and UGT1A3 to UGT1A10). Among them, five isomers (UGT1A1, UGT1A3, UGT1A4, UGT1A6 and UGT1A9) are expressed from the liver. The UGT1A gene family has different genetic polymorphism depending on people. It is known that several types of genetic polymorphism are present with respect to UGT1A1, and UGT1A3 to UGT1A10 genes (http://galien.pha.ulaval.ca/alleles/alleles.html). The polymorphism of UGT1A genes is significantly different between races. It has been confirmed that the activity of enzymes differs depending on the polymorphism, and the polymorphism is an important factor for determining sensitivity to drug treatment. UGT1A1*6 and UGT1A1*28 are related to Gilbert Syndrome (Monaghan G, Lancet, 347:578-81, 1996). Further, various functional variants which are related to various diseases have been reported.

As various types of genetic variants and haplotypes have been reported, the searching method should be efficient. The haplotypes can be analyzed by Arlequin, SNPalyze or other similar software. It would not be cost and time effective to analyze all single nucleotide polymorphism (SNP) for searching genetic variants of each haplotype.

In an effort to enhance efficiency, haplotype tagging SNPs (htSNPs) selection method can be provided. The htSNPs selection method is a method to select a minimum tagging set to accurately label each haplotype. If the selected SNPs are determined, all haplotypes can be predicted.

As for many genes, distribution of genetic polymorphism varies depending on races. Thus, it is necessary to check whether there are inherent genetic variants and haplotypes frequent in Koreans, and if so, how frequent they are, and how to select htSNPs depending on haplotypes. However, there have not been many studies on the genetic variants in the genes in Koreans, the haplotypes corresponding thereto and htSNPs selection according to each haplotype.

Recently, a method of determining 11 SNP through SNaPshot analysis centering on CYP2D6 genetic variants found mainly in Caucasians (Sistonen J et al., Clin Chem. 2005 July; 51 (7):1291-5), and CYP2D6 diagnosis chip of Roche or Jurilab Ltd. have been reported. However, they focus on CYP2D6 genetic variants found in Caucasians. Studies on diagnosing CYP2D6 genetic variants in Asians including Koreans are not sufficient.

Thus, the present inventors implemented a study to develop a method of determining variants of human CYP1A2, CYP2A6, CYP2D6, NR1I2 (=PXR) and UGT1A genes mainly found in Koreans accurately in a short time. The present invention provides a htSNP selection method for human CYP1A2, CYP2A6, CYP2D6, NR1I2 (=PXR) and UGT1A genetic variants mainly found in Koreans, to thereby prove availability of the selected htSNPs.

### DISCLOSURE OF INVENTION

Accordingly, it is an aspect of the present invention to provide an htSNP selection method for determining a haplotype of CYP1A2, CYP2A6, CYP2D6, NR1I2 (=PXR) and UGT1A genes found in Koreans.

Also, it is another aspect of the present invention to provide a method of determining a genotype of human CYP1A2, CYP2A6, CYP2D6, NR1I2 (=PXR) and UGT1A genes by using the htSNPs.

Further, it is another aspect of the present invention to provide a method of determining a genotype of a human CYP2D6 gene by using a kit including a gene chip.

Additional aspects and advantages of the general inventive concept will be set forth in part in the description which follows and, in part, will be obvious from the description, or may be learned by practice of the general inventive concept.

The foregoing and/or other aspects and advantages of the present invention are achieved by providing a method of selecting htSNPs of genes selected from human CYP1A2, CYP2A6, CYP2D6, PXR and UGT1A genes, comprising: collecting a biological sample from humans; extracting nucleic acid from the sample collected at operation (a); performing PCR (polymerase chain reaction) with a primer which amplifies a gene or a fragment thereof selected from human CYP1A2, CYP2A6, CYP2D6, PXR and UGT1A genes, by using the nucleic acid extracted at operation as a template; determining presence of variants from a genetic sequence of PCR products obtained at operation (c); determining a haplotype from the genetic sequence of the PCR products that is determined to have variants at operation (d); and sequencing the haplotype analyzed at operation (d) with SNPtagger software and selecting SNP.

The foregoing and/or other aspects and advantages of the present invention are achieved by providing a method of determining a genotype of a human CYP2A2 gene, comprising: (a) collecting a biological sample from subjects; (b) extracting a genomic DNA from the sample collected at operation (a); (c) performing PCR with a primer which amplifies a human CYP1A2 gene or a fragment thereof by using the genomic DNA extracted at operation (b) as a template; and (d) determining a presence of at least 11 variants of a CYP1A2 gene selected from −3860G>A, −3598G>T, −3594T>G, −3113G>A, −2847T>C, −2808A>C, −2603insA, −2467delT, −1708T>C, −739T>G, −163C>A, 1514G>A, 2159G>A, 2321G>C, 3613T>C, 5347C>T and 5521A>G in a genetic sequence of a PCR product obtained at operation (c).

The foregoing and/or other aspects and advantages of the present invention are achieved by providing a method of detecting a variant in a CYP1A2 promoter gene, comprising: (a) collecting a biological sample from subjects; (b) extracting a genomic DNA from the sample collected at operation (a); (c) performing PCR with a primer which amplifies a promoter region of a human CYP1A2 gene by using the genomic DNA obtained at operation (b) as a template; (d) determining a presence of CYP1A2 genetic variants including −3860G>A, −3598G>T, −3594T>G, −3113G>A, −2847T>C, −2808A>C, −2603insA, −2467delT, −1708T>C, −739T>G and −163C>A in a genetic sequence of a PCR product obtained at operation (c).

The foregoing and/or other aspects and advantages of the present invention are achieved by providing a method of determining a genotype of a human CYP2A6 gene, comprising: (a) collecting a biological sample from subjects; (b) extracting nucleic acid from the sample collected at operation (a); (c) performing PCR with a primer which amplifies a human CYP2A6 gene or a fragment thereof by using the nucleic acid obtained at operation (b) as a template; and (d) determining a presence of CYP2A6 genetic variants selected from −48T>G, 13G>A, 567C>T, 2134A>G, 3391T>C, 6458A>T, 6558T>C, 6582G>T, 6600G>T and 6091C>T in a genetic sequence of a PCR product obtained at operation (c).

The foregoing and/or other aspects and advantages of the present invention are achieved by providing a method of determining a genotype of a human CYP2D6 gene, comprising: (a) collecting a biological sample from humans; (b) extracting nucleic acid from the sample collected at operation (a); (c) performing PCR with a primer which amplifies a human CYP2D6 gene or a fragment thereof by using the nucleic acid obtained at operation (b) as a template; and (d) determining a presence of at least 11 variants a CYP2D6 gene including one from −1426C>T, 100C>T and 1039C>T; one from −1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 2988G>A; 4125-4133insGTGCCCACT; 2D6 deletion; and 2D6 duplication.

The foregoing and/or other aspects and advantages of the present invention are achieved by providing a method of determining a genotype of a PXR gene, comprising: (a) collecting a biological sample from humans; (b) extracting nucleic acid from the sample collected at operation (a); (c) performing PCR with a primer which amplifies a human PXR gene or a fragment thereof by using the nucleic acid obtained at operation (b) as a template; and (d) investigating presence of genetic variants of the PXR gene selected from −25385C>T, −24113G>A, 7635A>G, 8055C>T, 11156A>C and 11193T>C in a genetic sequence of a PCR product obtained at operation (c).

The foregoing and/or other aspects and advantages of the present invention are achieved by providing a method of determining a functional variant of UGT1A genes, comprising: (a) collecting a biological sample from humans; (b) extracting nucleic acid from the sample collected at operation (a); (c) individually amplifying human UGT1A genes by using the nucleic acid extracted at operation (b); and (d) sequencing the genes amplified at operation (c) and determining a presence of a functional variant in the UGT1A genes selected from −39(TA)6>(TA)7, 211G>A, 233C>T and 686C>A of a UGT1A1 gene; 31T>C, 133C>T and 140T>C of a UGT1A3 gene; 31C>T, 142T>G and 292C>T of a UGT1A4 gene; 19T>G, 541A>G and 552A>C of a UGT1A6 gene; 387T>G, 391C>A, 392G<A, 622T>C and 701T>C of a UGT1A7 gene; and −118T9>T10, 726T>G and 766G>A of a UGT1A9 gene.

The foregoing and/or other aspects and advantages of the present invention are achieved by providing a method of determining polymorphism of UGT1A genes related to sensitivity to irinotecan, comprising: (a) collecting a biological sample from humans; (b) extracting nucleic acid from the sample collected at operation (a); (c) amplifying human UGT1A genes by using the nucleic acid extracted at operation (b); and (d) sequencing the human UGT1A genes amplified at operation (c) and determining a presence of variants in the UGT1A genes selected from 211G>A, 233C>T and 686C>A of a UGT1A1 gene; 19T>G, 541A>G and 552A>C of a UGT1A6 gene; and −118T9>T10, 726T>G and 766G>A of a UGT1A9 gene.

The foregoing and/or other aspects and advantages of the present invention are achieved by providing a method of determining a genotype of a human CYP2D6 gene by using a gene chip, comprising: (a) extracting a gene to be investigated and obtaining PCR products including a circumference of SNP to be identified by performing multiplex PCR; (b) performing ASPE reaction by using an ASPE (allele specific primer extension) primer which identifies a specific base of allele; (c) mixing the reaction product to a gene chip; and (d) analyzing the gene chip.

The foregoing and/or other aspects and advantages of the present invention are achieved by providing a SNaPshot genotyping kit to determine a genotype of a CYP2D6 gene and a gene chip which has a Zip Code oligonucleotide chip to determine SNP.

The biological sample according to the present invention includes blood, skin cells, mucous cells and hair of subjects, and preferably blood.

The nucleic acid according to the present invention may include DNA or RNA, preferably DNA and more preferably genomic DNA.

The variants according to the present invention will be described as follows.

The term “aN>M” or “NaM” (a is a positive number, N and M are A, C, T or G individually) in the present invention refers that an N base in “a”th is replaced with an M base in genetic sequences. The term “ainsN” or “ade1N” (a is a positive number, and N is A, C, T or G) is that one more N base is inserted or deleted with respect to the “a”th in the genetic sequence.

For example, “−1548C>T” variant is that a C base is replaced with a T base in −1584^{th} of the genetic sequence.

“2573insC” variant is that a C base is inserted (added) to the 2573th in the genetic sequence. “4125-4133insGTGCCCACT” variant is that nine bases of GTGCCCACT are inserted to 4125^{th} to 4133th bases of a human CYP2D6 gene.

“2D6 deletion” variant is that the entire human CYP2D6 gene is deleted from a chromosome.

Further, “2D6 duplication” variant is that at least two human CYP2D6 genes are duplicated in the same chromosome.

As described above, the present invention provides a method of analyzing functional variants or polymorphism related to drug sensitivity of CYP1A2, CYP2A6, CYP2D6, PXR and UGT1A genes by using an optimal search set based on polymorphism of Korean CYP1A2, CYP2A6, CYP2D6, PXR and UGT1A genes that have not been checked up to now. The present invention may applicable to determine a genotype of CYP1A2, CYP2A6, CYP2D6, PXR and UGT1A genes of Asians including Japanese and Chinese similar to Koreans in genetic property, as well as Koreans.

### BRIEF DESCRIPTION OF THE DRAWINGS

The above and/or other aspects of the present invention will become apparent and more readily appreciated from the following description of the embodiments, taken in conjunction with the accompany drawings of which:

FIG. 1 illustrates a location of one variant in a CYYP1A2 gene that is determined for the first time according to the present invention, in a genetic sequence of a CYP1A2 gene;

FIGS. 2 to 6 are an example of htSNP combinations of a CYP1A2 gene selected according to the present invention;

FIGS. 7 to 14 illustrate results of variants of CYP1A2 promoter gene which are functional variants of the CYP1A2 gene selected according to the present invention (here, axis X refers to movement according to the molecule amount of primers and axis Y refers to height of each peak),

FIG. 7 illustrates a wild type SNP of a CYP1A2 promoter,

FIG. 8 illustrates −3860G>A (CYP1A2*1C), −2467delT (CYP1A2*1D) and −163C>A (CYP1A2*1F) variants in the CYP1A2 promoter which are placed in a hetero variant having one variant and one wild type among a double-stranded DNA,

FIG. 9 illustrates 3860G>A (CYP1A2*1C), −2467delT (CYP1A2*1D) and −163C>A (CYP1A2*1F) of the CYP1A2 promoter which are placed in a homo variant having two variants of a double-stranded DNA,

FIG. 10 illustrates −163C>A (CYP1A2*1F) and −2808A>C of the CYP1A2 promoter which are placed in a hetero variant,

FIG. 11 illustrates −163C>A (CYP1A2*1F) of the CYP1A2 promoter which is placed in a homo variant, and illustrates −2467delT (CYP1A2*1D), −739T>G (CYP1A2*1E), −3598G>T, −3113G>A, −2847T>C and −1708T>C which are placed in a hetero variant,

FIG. 12 illustrates −163C>A (CYP1A2*1F) of the CYP1A2 promoter which is placed in a homo variant, and illustrates −2467delT (CYP1A2*1D), −3598G>T and −2847T>C which are placed in a hetero variant,

FIG. 13 illustrates −163C>A (CYP1A2*1F), −2467delT (CYP1A2*1D) and −3594T>G of the CYP1A2 promoter which are placed in a hetero variant,

FIG. 14 illustrates −163C>A (CYP1A2*1F) of the CYP1A2 promoter which is placed in a homo variant, and illustrates −3860G>A (CYP1A2*C), −2467delT (CYP1A2*1D) and −2603insA which are placed in a hetero variant;

FIG. 15 illustrates types and frequencies of haplotypes in Koreans with respect to CYP2A6 used for selecting htSNP combinations according to the present invention;

FIGS. 16 to 21 exemplify the htSNP combinations of the CYP2A6 gene selected according to the present invention,

FIG. 16 illustrates selection of htSNP combination for determining a haplotype of CYP2A6 gene by adding six functional variants and three genetic variants having alleged functionality to targets of genetic variants examination,

FIG. 17 illustrates selection of htSNP combination for determining a haplotype of a CYP2A6 gene including eight variants having amino acid substation, three variants tagging CYP2A6 gene deletion and six frequent CYP2A6 genetic variants,

FIG. 18 illustrates selection of another htSNP combination for determining a haplotype of a CYP2A6 gene including eight variants having amino acid substation, three variants tagging CYP2A6 gene deletion and six frequent CYP2A6 genetic variants,

FIG. 19 illustrates selection of another htSNP combination for determining a haplotype of a CYP2A6 gene including eight variants having amino acid substation, three variants tagging CYP2A6 gene deletion and six frequent CYP2A6 genetic variants,

FIG. 20 illustrates selection of another htSNP combination for determining a haplotype of a CYP2A6 gene including eight variants having amino acid substation, three variants tagging CYP2A6 gene deletion and six frequent CYP2A6 genetic variants,

FIG. 21 illustrates selection of another htSNP combination for determining a haplotype of CYP2A6 gene including eight variants having amino acid substation, three variants displaying CYP2A6 gene deletion and six frequent CYP2A6 gene variants,

FIGS. 22 to 30 illustrate results of SNaPshot analysis with respect to the selected htSNP combinations and a combination of CYP2A6 functional genetic variants,

FIG. 22 illustrates −48T>G, 2134A>G and 6558T>C variants of CYP2A6 gene which are placed in a hetero variant having one variant and one wild type among a double-stranded DNA,

FIG. 23 illustrates 567C>7 variant of the CYP2A6 gene which is placed in a hetero variant having one variant and one wild type among a double-stranded DNA,

FIG. 24 illustrates 6458A>T and 6558T>C variants of a CYP2A6 gene which are placed in a hetero variant having one variant and one wild type of a double-stranded DNA,

FIG. 25 illustrates −48T>G, 13G>A and 6558T>C variants of a CYP2A6 gene which are placed in a hetero variant having one variant and one wild type among a double-stranded DNA,

FIG. 26 illustrates 3391T>C variant of a CYP2A6 gene which is placed in a hetero variant having one variant and the other one deleted among a double-stranded DNA,

FIG. 27 illustrates −48T>G and 2134A>G variants of a CYP2A6 gene which are placed in a hetero variant having one variant and the other one deleted among a double-stranded DNA,

FIG. 28 illustrates −48T>G, 6558T>C and 6600G>T variants of a CYP2A6 gene which are placed in a hetero variant having one variant and one wild type among a double-stranded DNA,

FIG. 29 illustrates 6458A>T variant of a CYP2A gene which is placed in a hetero variant having one variant and the other one deleted among a double-stranded DNA,

FIG. 30 illustrates 6558T>C and 6582G>T variants of a CYP2A6 gene which are placed in a hetero variant having one variant and one wild type among a double-stranded DNA;

FIGS. 31 and 32 illustrate SNaPshot analysis which is performed to additionally determine CYP2A6 gene deletion other than the genetic variants in FIGS. 22 to 30, together with the gene investigation in FIGS. 22 to 30,

FIG. 31 illustrates a CYP2A6 gene which is present in a homologous chromosome,

FIG. 32 illustrates a CYP2A6 gene which is not present in one chromosome and has only one gene;

FIG. 33 illustrates conjugation of a part of a CYP2A6 gene and a CYP2A7 gene;

FIGS. 34 to 39 illustrate htSNP combination of a CYP2D6 gene selected according to the present invention,

FIGS. 40 and 41 illustrate results of SNaPshot analysis of one of htSNP combinations in a CYP2D6 gene selected according to the present invention;

FIG. 42 illustrates a process of determining a genotype of a CYP2D6 gene by using a gene chip;

FIG. 43 illustrates a probe on the gene chip for a CYP2D6 gene;

FIG. 44 illustrates amplification of a CYP2D6 gene by using a long PCR;

FIG. 45 illustrates an ASPE reaction process;

FIG. 46 illustrates a gene chip which shows analysis results of variants in a CYP2D6 gene according to an exemplary embodiment 12;

FIG. 47 illustrates a htSNP combination of a PXR gene selected according to the present invention;

FIGS. 48 to 50 illustrate results of searching functional variants in a PXR gene selected according to the present invention (here, axis X refers to movement according to the molecule amount of each primer and axis Y refers to height of each peak),

FIG. 48 illustrates functional variants −25385C>T, −24113G>A, 7635A>G, 8055C>T, 11156A>C and 11193T>C of a PXR gene which are all wild types;

FIG. 49 illustrates functional variants −25385C>T, −24113G>A, 7635A>G, 8055C>T, 11156A>C and 11193T>C of a PXR gene which are placed in a hetero variant having one variant and one wild type in a double-stranded DNA;

FIG. 50 illustrates functional variants −25385C>T, −24113G>A, 7635A>G, 8055C>T, 11156A>C and 11193T>C of a PXR gene which are placed in a homo variant having two variants in a double-stranded DNA;

FIGS. 51 to 54 illustrate analysis results of functional variants of UGT1A genes of 50 Koreans (here, axis X refers to a position of SNP, axis Y refers to height of each peak, red color is T, black color C, blue color G and green color A);

FIG. 51 illustrates analysis result of functional variants in UGT1A1 (a) and UGT1A3 (b) genes;

FIG. 52 illustrates analysis result of functional variants in UGT1A4 (a) and UGT1A6 (b) genes;

FIG. 53 illustrates analysis result of functional variants in UGT1A7 gene;

FIG. 54 illustrates analysis result of functional variants in a UGT1A9 gene;

FIG. 55 illustrates analysis result of polymorphism related to sensitivity to irinotecan of UGT1A1, UGT1A6 and UGT1A9 genes of 50 Koreans;

(a) 211G>A, 233C>T and 686C>A from a UGT1A1 gene; 19T>G, 541A>G and 552A>C from a UGT1A6 gene; and 726T>G and 766G>A from a UGT1A9 gene which are all wild types,

(b) wild types 211G>A and 233C>T, a hetero type 686C>A from a UGT1A1 gene; hetero types 19T>G and 552A>C, a wild type 541A>G from a UGT1A6 gene; and wild types 726T>G and 766G>A from a UGT1A9 gene,

(c) wild types 211G>A, 233C>T and 686C>A from a UGT1A1 gene; hetero type 19T>G, 541A>G and 552A>C from a UGT1A6 gene; and a hetero type 726T>G and a wild type 766G>A from a UGT1A9 gene, and

(d) hetero types 211G>A and 233C>T, and a wild type 686C>A from a UGT1A1 gene; hetero types 19T>G, 541A>G and 552A>C from a UGT1A6 gene; and wild types 726T>G and 766G>A from a UGT1A9 gene.

### MODES FOR CARRYING OUT THE INVENTION

Hereinafter, exemplary embodiments of the present invention will be described with reference to accompanying drawings, wherein like numerals refer to like elements and repetitive descriptions will be avoided as necessary.

The present invention is provided to determine a genotype of a CYP1A2 gene found in Koreans through variant analysis of Korean CYP1A2 gene, select htSNPs as an optimal tagging set of each haplotype and confirm its availability. Also, the present invention is provided to determine a novel haplotype of a human CYP1A2 gene.

A method of selecting htSNPs of a human CYP1A2 gene according to the present invention is as follows:

(a) collecting a biological sample from subjects;

(b) extracting nucleic acid from the sample collected at operation (a);

(c) performing PCR with a primer which amplifies a human CYP1A2 gene or a fragment thereof by using the nucleic acid extracted at operation (b) as a template;

(d) determining presence of variants from a genetic sequence of PCR products obtained at operation (c);

(e) sequencing the PCR products that is determined to have variants at operation (d) with SNPtagger software.

The method of extracting the nucleic acid from the sample collected at operation (a) is not limited, and is known in the art. Alternatively, extraction kits may be used to extract the nucleic acid. For example, DNA or RNA extraction kits which are manufactured by Qiagen (USA) and Stratagene (USA) may be used. If RNAs are extracted from the kits, cDNA is manufactured by reverse transcription to be used.

The fragment of the human CYP1A2 gene at operation (c) refers to a fragment which includes known variants of a human CYP1A2 gene, e.g. single nucleotide polymorphism (SNP). The primer which amplifies the human CYP1A2 gene or the fragment thereof may be designed based on a genetic sequence of a human CYP1A2 gene or a fragment thereof, and may be selected from primers having references 2 to 31, but not limited thereto.

The variants at operation (d) include SNP, gene deletion and gene duplication, but not limited thereto. For example, the variants may include 17 variants as in Table 5.

The sequencing method is not limited, and may be known in the art. For example, an automated DNA sequencer may be used or pyrosequencing may be performed to determine the genetic sequence. The pyrosequeuncing is a known SNP determining method which is used in DNA sequencing, and is a method of detecting light expression from inorganic pyrophosphate (PPi) discharged while DNA is polymerized. The DNA sequencing may be performed by using primers from references 32 to 61, but not limited thereto. The presence of variants at operation (d) may be determined by comparing genetic sequences of a wild type CYP1A2 gene. The genetic sequences of the wild type CYP1A2 gene, e.g. genetic sequences of reference 1 (GenBank accession No.: NT_{—}010194) or each genetic sequence of CYP1A2 genotypes known in the art may be used (Drug Metab. Pharmacokinet, 2005, 20 (1):24-33).

The frequencies and types of haplotypes may be estimated by using a technical program known in the art or a program that is sold in the market. For example, Haploview which is distributed free of charge, or SNPAlyze which is a commercialized program may be used. The Haploview software are known in the art. More preferably, the software may be downloaded from http://www.broad.mit.edu/mpg/haploview.

The method according to the present invention may additionally include repetition of operations (a) to (d). To determine variant phases of a CYP1A2 gene and haplotypes thereof within a particular group such as races or patients, operation (e) may be performed after frequencies of CYP1A2 genotypes are examined and frequent CYP1A2 genotypes are selected from the group.

At operation (e), haplotype data of CYP1A2 gene are analyzed with the SNP tagger software to select htSNPs. The SNP tagger software are known in the art, e.g. Genehunter, Merlin, Allegro, SNPHAP, htSNP finder (PCA based). More preferably, the software may be downloaded from http://www.well.ox.ac.uk/˜xiayi/haplotype or http://slack.ser.man.ac.uk/progs/htsnp.html.

The selected htSNPs may be verified to improve accuracy to thereby determine diplotypes. As a genotype of humans is determined by double-stranded chromosomes, the genotype is decoded to determine two haplotype combinations. If several SNP are analyzed simultaneously, a combination of a particular haplotype may be the same as that of another haplotype. If the genotype is decoded with the diagnosis developed according to the present invention, it should be verified whether to determine the genotype accurately. Such verification may be performed by analyzing whether the genotype is correctly decoded from the gene analysis result, using Matlab (The math Works Inc., US).

According to an exemplary embodiment of the present invention, variants in a CYP1A2 gene of Koreans are investigated first to select htSNPs of CYP1A2 genotypes found in Koreans. As a result, a total of 17 SNP are found in the CYP1A2 gene of Koreans (refer to Table 5). One of 17 SNP (−2603insA) is novel.

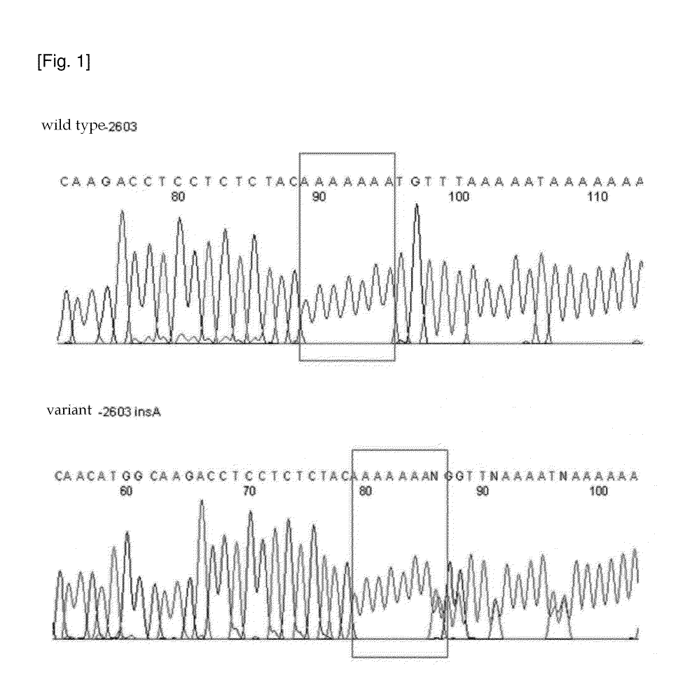

The single SNP which is provided for the first time according to the present invention includes a one variant and one wild type among a double-stranded DNA (Refer to FIG. 1).

According to another exemplary embodiment of the present invention, a haplotype of 17 SNPs found in Koreans is determined. As a result, the present invention determined a haplotype of a CYP1A2 gene never found before in Koreans (refer to Table 6) and a genotype based thereon. For example, a haplotype 2 (CYP1A2*1L) of the CYP1A2 gene in Table 6 refers to a genotype which has a SNP in bases −3860, −2467 and −163 from the genetic sequence of the CYP1A2 gene. More specifically, the genotype has a SNP of −3860G>A, −2467T>delT (−2467delT) and 163C>A.

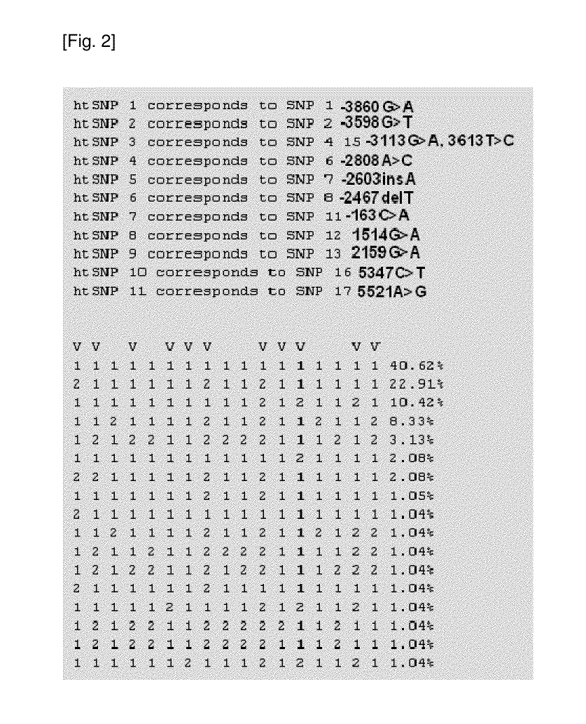

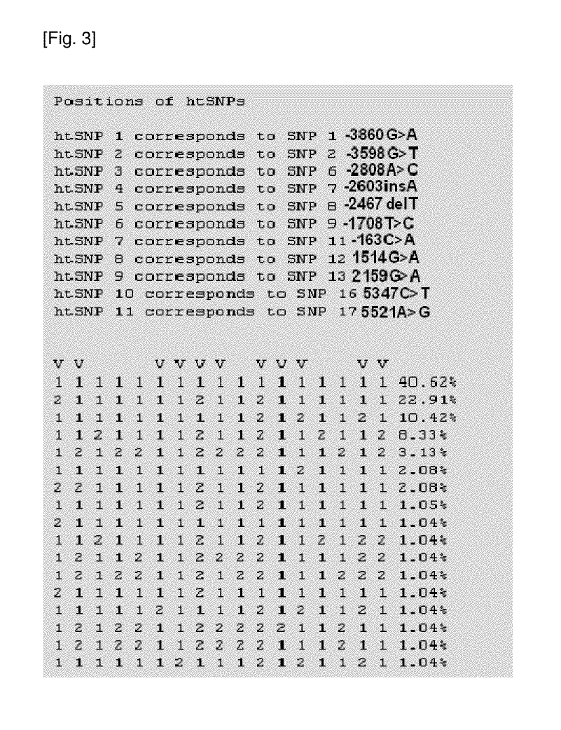

According to another exemplary embodiment of the present invention, a haplotype which is determined by genetic sequences having variants in a CYP1A2 gene is analyzed by SNPtagger software to thereby select htSNPs, a minimum marker to 17 haplotypes of variants in the CYP1A2 gene found in Koreans. An example of combination of htSNPs selected according to the present invention is shown in FIGS. 2 to 6.

The htSNP combination selected according to the present invention may be used to determine the haplotype of a human CYP1A2 gene. Thus, the present invention provides a method of determining a haplotype of a human CYP1A2 gene. The method includes following steps:

(a) collecting a biological sample from subjects;

(b) extracting nucleic acid from the sample collected at operation (a);

(c) performing PCR with a primer which amplifies a human CYP1A2 gene or a fragment thereof by using the nucleic acid obtained at operation (b) as a template; and

(d) determining presence of variants in the CYP1A2 gene selected from −3860G>A, −3598G>T, −3594T>G, −3113G>A, −2847T>C, −2808A>C, −2603insA, −2467delT, −1708T>C, −739T>G, −163C>A, 1514G>A, 2159G>A, 2321G>C, 3613T>C, 5347C>T and 5521A>G in a genetic sequence of PCR products obtained at operation (c).

The method of extracting the nucleic acid at operation (b) is the same as that described above.

As described above, the fragment of the human CYP1A2 gene refers to a fragment which includes a known SNP of the human CYP1A2 gene. The primer which may be used at operation (c) is not limited, and may be selected from references 2 to 31.

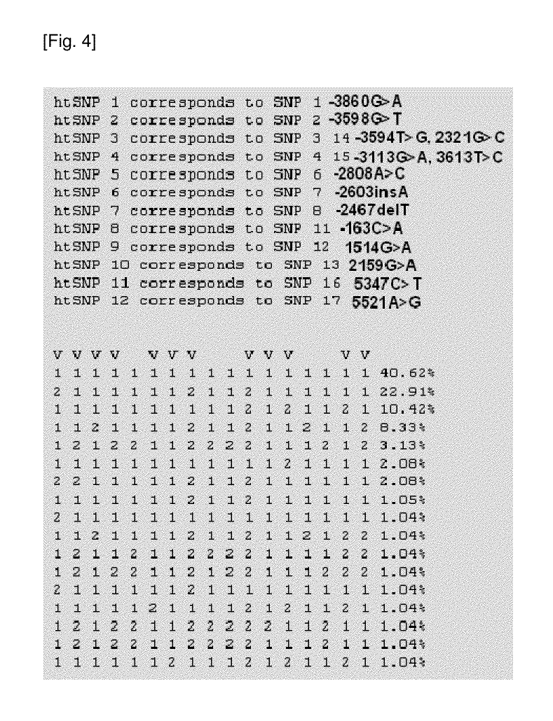

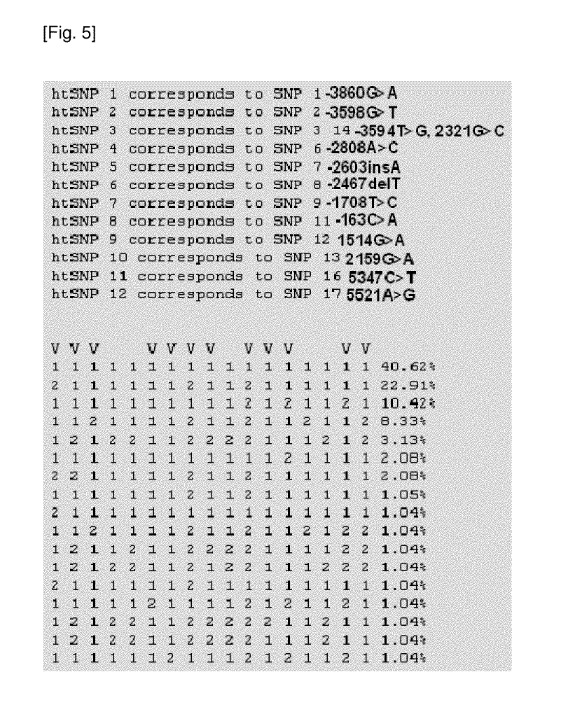

The SNP which is investigated at operation (d) may be selected from htSNPs in FIGS. 4 to 6. The presence of SNP may be investigated from −3860G>A, −3598G>T, −3113G>A, −2808A>C, −2603insA, −2467delT, −163C>A, 1514G>A, 2159G>A, 5347C>T and 5521A>G in FIG. 4; −3860G>A, −3113G>A, −2808A>C, −2603insA, −2467delT, −739T>G, −163C>A, 1514G>A, 2159G>A, 5347C>T and 5521A>G in FIG. 5; and −3860G>A, −3598G>T, −3594T>G, −3113G>A, −2808A>C, −2603insA, −2467delT, −163C>A, 1514G>A, 2159G>A, 5347C>T and 5521A>G in FIG. 6 or −3860G>A, −3598G>T, 2321G>C, −3113G>A, −2808A>C, −2603insA, −2467delT, −163C>A, 1514G>A, 2159G>A, 5347C>T and 5521A>G.

The SNP which is examined at operation (d) is based on variants in the CYP1A2 gene found in Koreans, and is very specific to determine a haplotype and a genotype of the CYP1A2 gene of Koreans.

The SNP of the CYP1A2 gene in the genetic sequence of the PCR products at operation (d) may be determined by polymorphism analysis method known in the art. Preferably, the SNP may be determined by SNaPshot analysis (refer to [Peter M. Vallone, et al., Int J Legal Med, 2004, 118:147-157]), electrophoretic analysis or a combination thereof, and more particularly by SNaPshot analysis.

The SNaPshot analysis refers to a method of determining a genotype through PCR reaction with a primer having an annealed genetic sequence (excluding SNP region) around a SNP position and ddNTP. The SNaPshot which is used in the present invention is designed and manufactured by a known method based on the SNP of the CYP1A2 gene investigated at operation (c). The SNaPshot used may vary as long as it has a base right next to the SNP position as 3′end, includes an annealed genetic sequence adjacent to the SNP position and has a T base added to a 5′end. More specifically, a primer may be selected from references 64 to 74. Preferably, the annealed genetic sequence adjacent to the SNP position is approximately 20 bp long. If several SNPs are determined simultaneously, the length of the T base at the 5′end of the SNaPshot primers is designed to vary. For example, five T bases are added to the 5′ end so that the primers differ in size, thereby varying the length of the PCR products. Then, the SNaPshot primers are coupled with ddNTP complementary to each SNP. Those composites differ in size depending on the SNP. Thus, several SNPs can be determined simultaneously.

To determine whether the genotyping results using the SNaPshot analysis are correct, another method of determining the genotype is performed. Another method is not limited, and preferably includes an automated DNA sequencing or pyrosequencing.

Eleven SNPs among 17 variants in the CYP1A2 gene found in Koreans are located in a promoter region. The eleven SNPs include −2603insA variant determined for the first time by the present invention. Thus, the present invention provides a method of determining variants in a human CYP1A2 promoter gene. The method includes following steps:

(a) collecting a biological sample from subjects;

(b) extracting a genomic DNA from the sample collected at operation (a);

(c) performing PCR with a primer which amplifies a promoter region of a human CYP1A2 gene by using the genomic DNA obtained at operation (b) as a template; and

(d) determining presence of variants in the CYP1A2 gene including −3860G>A, −3598G>T, −3594T>G, −3113G>A, −2847T>C, −2808A>C, −2603insA, −2467delT, −1708T>C, −739T>G and −163C>A in a genetic sequence of PCR products obtained at operation (c).

The method of extracting the genomic DNA at operation (b) is the same as described above.

The primer which amplifies the promoter region of the human CYP1A2 gene at operation (c) may vary as long as it amplifies SNPs from −3860G>A to −163C>A in reference 1, and more preferably from references 62 and 63.

The SNP of the CYP1A2 gene in the genetic sequence of the PCR products at operation (d) may be determined polymorphism analysis methods known in the art. Preferably, the SNP may be determined by SNaPshot analysis. The SNaPshot analysis used in the present invention may be performed by using the primer designed based on the 11 SNPs of the CYP1A2 gene. The SNaPshot primer which is used in the present invention may vary as long as it is designed to include a genetic sequence adjacent to a sequence excluding the SNP. More preferably, a primer which has a genetic sequence selected from references 64 to 74.

According to another exemplary embodiment of the present invention, based on 11 SNPs in the promoter region affecting activity of CYP1A2 enzymes, variants of the CYP1A2 promoter gene are detected with SNaPshot analysis. As a result, it was confirmed that the method according to the present invention may accurately detect the variants in the CYP1A2 promoter gene at high speed (refer to FIGS. 7 to 14).

2. CYP2A6

The present invention is provided to determine a genotype of a CYP1A2 gene found mainly in Koreans through variant analysis of Korean CYP2A6 gene, select htSNPs as an optimal tagging set of each haplotype and confirm its availability.

A method of selecting htSNPs of a human CYP2A6 gene according to the present invention is as follows:

(a) collecting a biological sample from subjects;

(b) extracting nucleic acid from the sample collected at operation (a);

(c) performing PCR with a primer which amplifies a human CYP1A2 gene or a fragment thereof by using the nucleic acid extracted at operation (b) as a template;

(d) determining presence of variants from a genetic sequence of PCR products obtained at operation (c);

(e) determining a haplotype from the genetic sequence of PCR products that is determined to have variants at operation (d); and

(f) selecting htSNPs by sequencing the haplotype determined at operation (e) with SNPtagger software (http://www.well.ox.ac.uk/˜xiayi/haplotype/).

The method of extracting the nucleic acid from the sample collected at operation (a) is not limited, and is known in the art. Alternatively, extraction kits may be used to extract the nucleic acid. For example, DNA or RNA extraction kits which are manufactured by Qiagen (USA) and Stratagene (USA) may be used. If RNAs are extracted, cDNA is manufactured by reverse transcription to be used.

The fragment of the human CYP2A6 gene at operation (c) refers to a fragment which includes known variants of a human CYP2A6 gene, e.g. single nucleotide polymorphism (SNP). The primer which amplifies a human CYP2A6 gene or a fragment thereof may be designed based on a genetic sequence of a human CYP2A6 gene or a fragment thereof, and may be selected from primers with references 76 to 89, but not limited thereto.

The variants at operation (d) includes SNP, gene deletion and gene duplication, but not limited thereto. For example, the variants may include 30 variants as in Table 15.

The method of determining the presence of the variants at operation (d) may include a variant detecting method which is known in the art. Preferably, a genetic sequence of a wild type CYP2A6 gene which is known in the art, e.g. genetic sequence of reference 75 (BenBank accession NO.: NC_{—}000019) or each genetic sequence of CYP2A6 genotypes which is known in the art may be compared with sequencing and electrophoretic analysis. Also, cut phases of a restriction enzyme of the wild type CYP2A6 gene may be compared through RFLP analysis. The deletion or duplication of the CYP2A6 gene may be determined by electrophoretic analysis of PCR products. The sequencing may be performed by automated DNA sequencer or by pyrosequencing.

The haplotype in the genetic sequence of the PCR products that is determined to have the variants at operation (e) may be determined by programs such as SNPAlyze, Haplotyper, Arlequin, etc.

The method according to the present invention may additionally include repetition of operations (a) to (d). To determine variant phases in a CYP2A6 gene within a particular group such as races or patients, operation (f) may be performed after frequencies of CYP1A2 genotypes are examined and frequent CYP1A2 genotypes are selected from the group.

At operation (f), the genetic sequence of the haplotype determined at operation (e) is analyzed by SNP tagger software to select htSNPs. The software which are used for selecting htSNPs include HapBlock, LDSelect, Haploview, htSNP, TagIT and tagSNPs as well as SNPtagger. The SNPtagger software are known in the art, and preferably may be downloaded from http://www.well.ox.ac.uk/˜xiayi/haplotype/. The selected htSNPs may be verified to improve accuracy to thereby determine diplotypes. The htSNPs may be verified by using Matlab (The Math Works Inc., USA).

According to an exemplary embodiment of the present invention, variants in the CYP2A6 gene found in Koreans are investigated first to select the htSNPs of the CYP2A6 genotype of Koreans. As a result, a total of 30 SNPs were found in the CYP2A6 gene of Koreans (refer to Table 15).

According to another exemplary embodiment of the present invention, haplotypes of 14 SNPs among the selected 30 SNPs are determined by SNPAlyze manufactured by DYNACOM, thereby determining a total of 19 haplotypes having a frequency of one percent and above. The 14 SNPs include eight variants causing amino acid substitution and having a functional genetic variant, and six frequent variants. The program used to determine the haplotypes is not limited to the SNPAlyze. Alternatively, various software known in the art may be used, e.g. Haplotyper (http://www.people.fas.harvard.edu/˜junliu/Haplo/docMain. htm), Arlequin (htt://lgb.unife.ch/arlequin) and SNP Analyzer manufactured by Istech (http://www.istech21.com/).

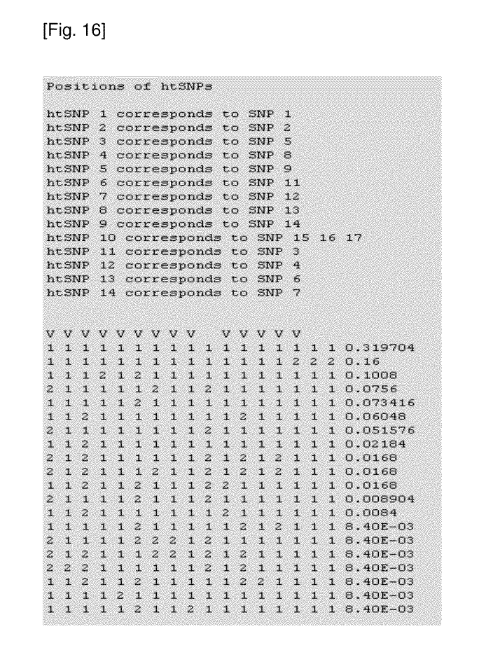

According to another exemplary embodiment of the present invention, the genetic sequence and frequency of 20 haplotypes including 19 haplotypes and one gene deletion are analyzed with SNPtagger software to select htSNPs, a minimum marker easily identifying the genotype of a CYP2A6 gene mainly found in Koreans. FIGS. 16 to 21 illustrate examples of htSNPs.

The htSNP combination selected according to the present invention may be used to determine a genotype of the human CYP2A6 gene. Thus, the present invention provides a method of determining a genotype of the human CYP2A6 gene. The method includes following steps:

(a) collecting a biological sample from subjects;

(b) extracting nucleic acid from the sample collected at operation (a);

(c) performing PCR with a primer which amplifies a human CYP2A6 gene or a fragment thereof by using the nucleic acid extracted at operation (b) as a template;

(d) determining presence of variants in the CYP2A6 gene selected from −48T>G, 13G>A, 567C>T, 2134A>G, 3391T>C, 6458A>T, 6558T>C, 6582G>T, 6600G>T, and 6091C>T in a genetic sequence of PCR products obtained at operation (c).

The method of extracting the nucleic acid at operation (b) is the same as described above.

As described above, the fragment of the human CYP2A6 gene refers to a fragment which includes known variants of a human CYP2A6 gene, e.g. single nucleotide polymorphism (SNP). The primer which may be used in operation (c) may include primers with references 90, 91, 120 and 130, but not limited thereto.

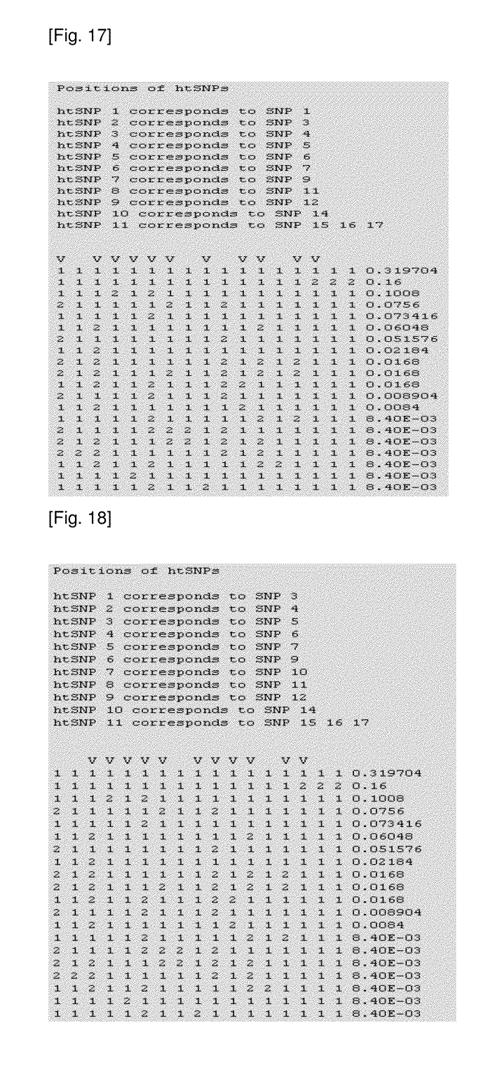

The variants at operation (d) may be selected from the htSNPs in FIGS. 16 to 21. For example, at operation (d), the variants may be determined from −48T>G; 22C>T; 567C>T; 2134A>G; 3391T>C; 6458A>T; 6558T>C; 6582G>T; 6600G>T; one from 6091C>T, 5971G>A and 5983T>G; and 13G>A; 51G>A; 1620T>C; and 1836G>T in FIG. 16. Also, the variants may be determined from −48T>G; 22C>T; 51G>A; 567C>T; 1620T>C; 1836G>T; 3391T>C; 6458A>T; 6558T>C; 6600G>T; and one from 6091C>T, 5971G>A and 5983T>G in FIG. 17.

As shown in FIG. 18, the variants may be determined from 22C>T; 51G>A; 567C>T; 1620T>C; 1836G>T; 3391T>C; 6354T>C; 6458A>T; 6558T>C; 6600G>T; and one from 6091C>T, 5971G>A and 5983T>G.

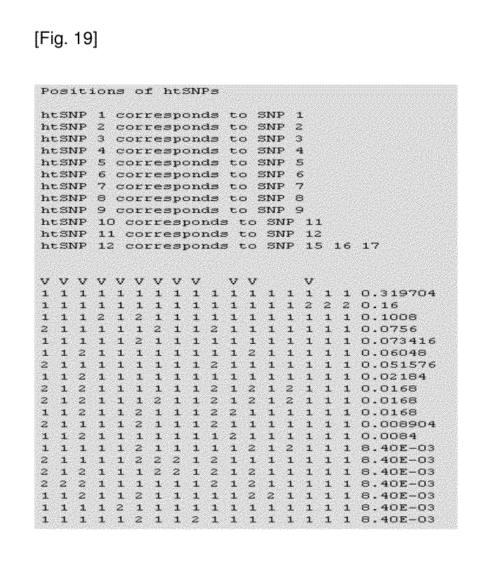

As shown in FIG. 19, the variants may be determined from −48T>G; 13G>A; 22C>T; 51G>A; 567C>T; 1620T>C; 1836G>T; 2134A>G; 3391T>C; 6458A>T; 6558T>C; and one from 6091C>T, 5971G>A and 5983T>G.

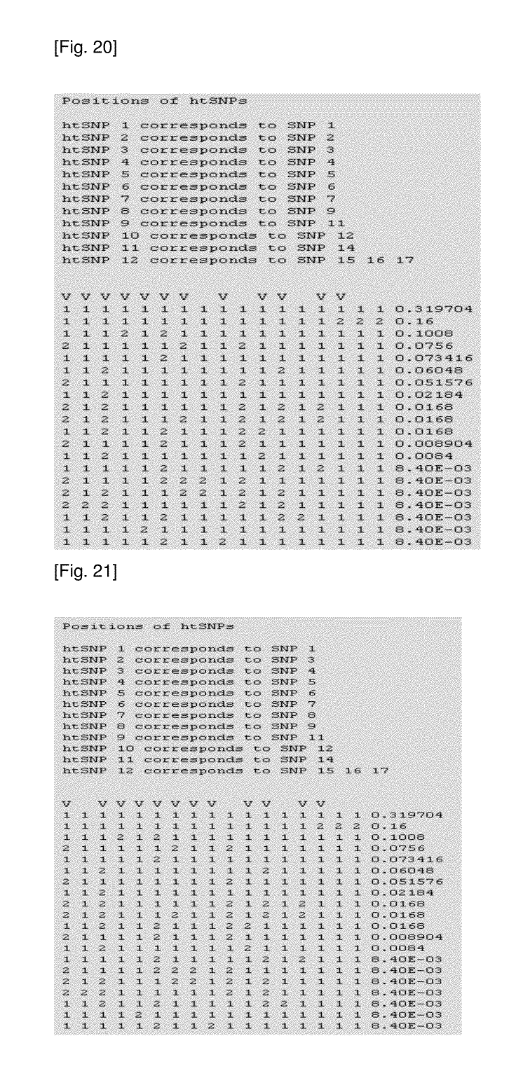

As shown in FIG. 20, the variants may be determined from −48T>G; 13G>A; 22C>T; 51G>A; 567C>T; 1620T>C; 1836G>T; 3391T>C; 6458A>T; 6558T>C; and one from 6091C>T, 5971G>A and 5983T>G.

Also, as shown in FIG. 21, the variants may be determined from −48T>G; 22C>T; 51G>A; 567C>T; 1620T>C; 1836G>T; 2134A>G; 3391T>C; 6458A>T; 6558T>C; 6600G>T; and one from 6091C>T, 5971G>A and 5983T>G. Preferably, the variants may be determined as shown in FIG. 16.

Among the variants in FIG. 16, the variant which proves functionality or has high potential functionality includes a variant which substitutes amino acid or causes gene deletion. Thus, to detect the functional CYP2A6 variant among the variants in FIG. 16, the amino acid substitution variants or gene deletion variant may be investigated among the variants in FIG. 16. The gene deletion is hardly detectable by the SNP. While the variant was searched to label the gene deletion, 6091C>T variant was found. The 6091C>T variant is a SNP which is specifically found in a chromosome deleting a CYP2A6 gene among PCR products amplified at operation (c), and can be used as a gene deletion-labeling variant. The combination of variants selected to determine functionality includes ten variants, i.e. −48T>G; 13G>A; 567C>T; 2134A>G; 3391T>C; 6458A>T; 6558T>C; 6582G>T; 6600G>T; and 6091C>T. 5971G>A and 5983T>G variants in the CYP2A6 gene may replace the 6091C>T variant to label gene deletion.

The variants which are investigated at operation (d) are based on variants in the CYP2A6 gene mainly found in Koreans. Thus, it is very specific to determine a haplotype and a genotype of the CYP2A6 gene of Koreans.

The variants in the CYP2A6 gene in the genetic sequence of the PCR products at operation (d) may be investigated by using polymorphism analysis methods known in the art. The SNaPshot analysis (refer to [Peter M. Vallone, et al., Int J Legal Med, 2004, 118:147-157]), electrophoretic analysis, or a combination thereof, and more particularly the SNaPshot analysis may be employed to investigate the variants.

The SNaPshot analysis refers to a method of determining a genotype through PCR reaction with a primer having an annealed sequence (excluding SNP region) around a SNP position and ddNTP. The SNaPshot which is used in the present invention is designed and manufactured by a known method based on the SNP of the CYP2A6 gene investigated at operation (d). The SNaPshot used may vary as long as it has a base right next to the SNP position as 3′end, includes an annealed genetic sequence adjacent to the SNP position and has a T base added to 5′end. More preferably, a primer may be selected from references 97 to 102. Preferably, the annealed genetic sequence adjacent to the SNP position is approximately 20 bp long. If several SNP are determined simultaneously, the length of the T base at 5′end of the SNaPshot primers is designed to vary. For example, five T bases are added to the 5′ end so that the primers differ in size, thereby varying the length of the PCR products. Then, the SNaPshot primers are coupled with ddNTP complementary to each SNP. Those composites differ in size depending on the SNP. Thus, several SNPs can be determined simultaneously.

Then, the genetic sequence of the PCR products which is amplified for the SNaPshot analysis may be analyzed by sequencing methods known in the art, preferably by automated DNA sequencing.

For example, a primer which has a genetic sequence selected from references 92 to 101 may be used to investigate the htSNP combination in FIG. 16 at operation (c). Preferably, all primers from references 92 to 101 may be used, but not limited thereto.

Then, the genetic sequence of the PCR products which is amplified for the SNaPshot analysis may be analyzed by sequencing methods known in the art, preferably by automated DNA sequencing.

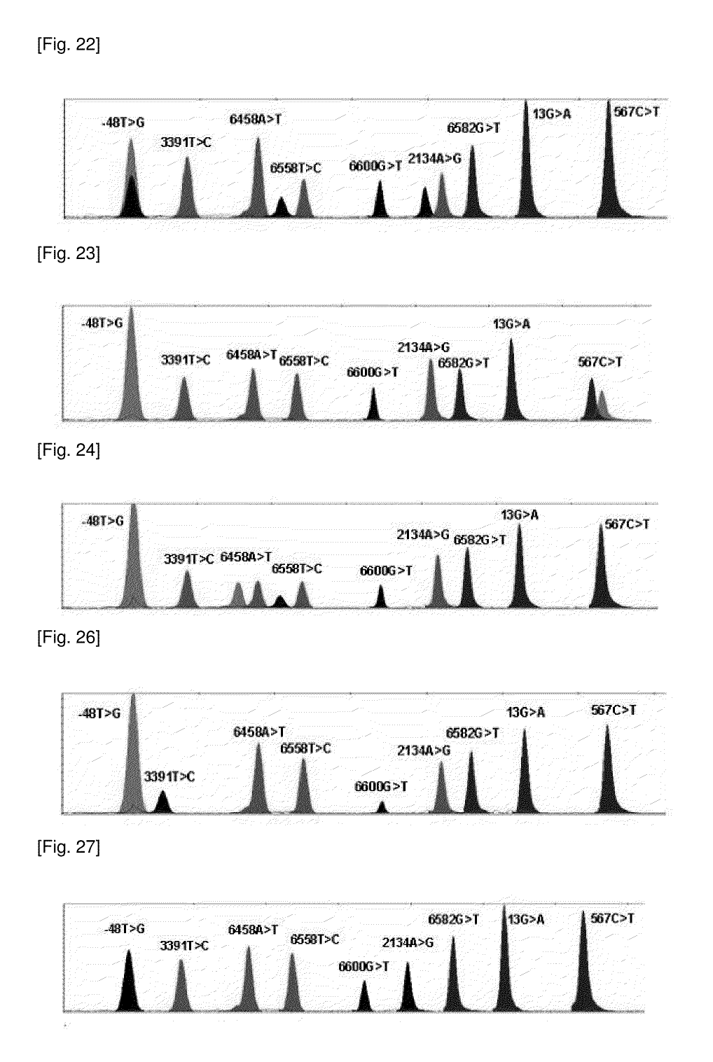

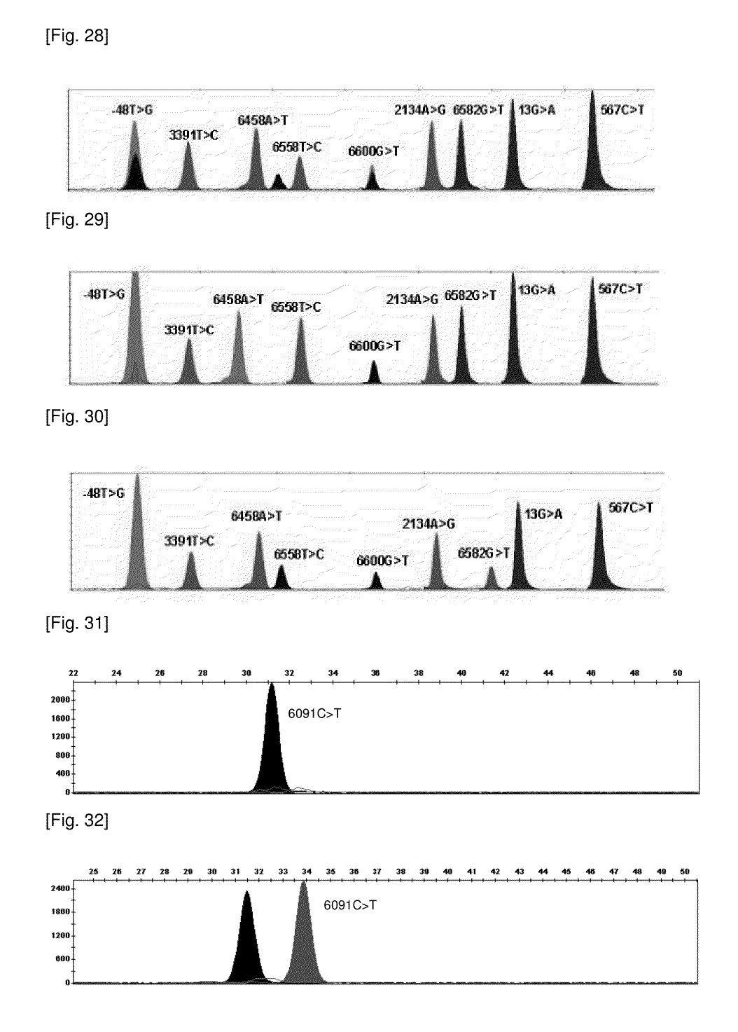

According to another exemplary embodiment of the present invention, availability of the selected htSNP combination is confirmed. The genetic sequence of the PCR products obtained at operation (c) is analyzed to perform the SNaPshot analysis by selecting ten functional or potentially-functional CYP2A6 variants among the htSNP combinations in FIG. 16. As a result, the method according to the present invention is confirmed to simultaneously determine the CYP2A6 genotypes found in Koreans at high speed (refer to FIGS. 22 to 32).

The genotypes of the CYP2A6 gene which can be determined by the method according to the present invention include −48T>G, 13G>A, 567C>T, 2134A>G, 3391T>C, 6458A>T, 6558T>C, 6582G>T, 6600G>T and 6091C>T. Each genotype and variants corresponding thereto are shown in FIGS. 22 to 32. For example, FIG. 22 illustrates a genotype which has −48T>G, 6558T>C and 2134A>G variants and seven wild types. FIG. 23 illustrates a genotype which has 567C>T variant and nine wild types. The CYP2A6*4 genotype includes 2A6 deletion variant. As the CYP2A6 gene is deleted from the human chromosomes, enzymes are not produced at all. If the CYP2A6 gene is deleted, the shape of the gene is a part of the CYP2A6 gene coupled with a part of the CYP2A7 gene. The deletion-specific variant may be determined by investigating the coupled genes described above.

3. CYP2D6

The present invention is provided to determine a genotype of a CYP2D6 gene found mainly in Koreans through variant analysis of Korean CYP2D6 gene, select htSNPs as an optimal tagging set of each haplotype and confirm its availability.

A method of selecting htSNPs of a human CYP2D6 gene according to the present invention is as follows:

(a) collecting a biological sample from humans;

(b) extracting nucleic acid from the sample collected at operation (a);

(c) performing PCR with a primer which amplifies a human CYP2D6 gene or a fragment thereof by using the nucleic acid extracted at operation (b) as a template;

(d) determining presence of variants from a genetic sequence of PCR products obtained at operation (c);

(e) determining a haplotype from the genetic sequence of PCR products that is determined to have variants at operation (d); and

(f) selecting htSNPs by sequencing the haplotype determined at operation (e) with SNPtagger software.

The method of extracting the nucleic acid from the sample collected at operation (a) is not limited, and is known in the art. Alternatively, extraction kits may be used to extract the nucleic acid. For example, DNA or RNA extraction kits which are manufactured by Qiagen (US) and Stratagene (US) may be used. If RNAs are extracted, cDNA is manufactured by reverse transcription to be used.

The fragment of the human CYP2D6 gene at operation (c) refers to a fragment which includes known variants of a human CYP2D6 gene, e.g. single nucleotide polymorphism (SNP). The primer which amplifies the human CYP2D6 gene or the fragment thereof may be designed based on a genetic sequence of a human CYP2D6 gene or a fragment thereof. For example, the primer may include a genetic sequence selected from reference 106, reference 107, references from 121 to 127, references from 129 to 136, reference 138, reference 139, reference 140 and reference 150, but not limited thereto.

The variants at operation (d) include SNP, gene deletion and gene duplication, but not limited thereto. For example, the variants may include 33 variants as in Table 34.

The method of determining the presence of the variants at operation (d) may include a variant detecting method which is known in the art. Preferably, genetic sequencing, electrophoretic analysis and RFLP analysis may be performed to determine the variants. The genetic sequencing may be performed by an automated DNA sequencer or pyrosequencing. The pyroseqeuncing is a known SNP determining method which is used in DNA sequencing, and is a method of detecting light expression from inorganic pyrophosphate (PPi) discharged while DNA is polymerized.

The presence of the variants at operation (d) may be determined by comparing genetic sequences of a wild type CYP2D6 gene. The genetic sequences of the wild type CYP2D6 gene are known in the art. For example, a genetic sequence of a reference 105 (GenBank accession No. AY545216) or each genetic sequence of CYP2D6 genotypes known in the art may be used (GenBank accession NO. M33388, http://www.cypalleles.ki.se/cyp2d6.htm). Also, cut phases of a restriction enzyme of the wild type CYP2D6 gene may be compared by performing RFLP analysis. The deletion or duplication of the CYP2D6 gene may be determined by electrophoretic analysis of PCR products.

The haplotype in the genetic sequence of the PCR products that is confirmed to have variants at operation (d) may be determined by full sequencing.

The method according to the present invention may additionally include repetition of operations (a) to (d). To determine variant phases of a CYP2D6 gene within a particular group such as races or patients, operation (f) may be performed after frequencies of CYP2D6 genotypes are examined and frequent CYP2D6 genotypes are selected from the group.

At operation (f), the genetic sequence of the haplotype determined at operation (e) is analyzed with the SNPtagger software to select htSNPs. The SNPtagger software are known in the art, e.g. Genehunter, Merlin, Allegro, SNPHAP, htSNP finder (PCA based), and more preferably, downloaded from http://www.well.ox.ac.uk/˜xiayi/haplotype or http://slack.ser.man.ac.uk/progs/htsnp.html.

The selected htSNPs may be verified to improve accuracy to thereby determine diplotypes. As a genotype of humans is determined by double-stranded chromosomes, the genotype is decoded to determine two haplotype combinations. If several SNP are determined simultaneously, a combination of a particular haplotype may be the same as that of another haplotype. If the genotype is decoded with the diagnosis developed according to the present invention, it should be verified whether to determine the genotype accurately. Such verification may be performed by analyzing whether the genotype is decoded from the gene analysis result, using Matlab (The math Works Inc., US).

According to an exemplary embodiment of the present invention, variants in the CYP2D6 gene found in Koreans are investigated first to select the htSNPs of the CYP2D6 genotype of Koreans. As a result, 33 variants and 12 haplotypes (genotypes) corresponding thereto were found in the CYP2D6 gene of Koreans (refer to Tables 34 and 35).

According to another exemplary embodiment of the present invention, 12 CYP2D6 genotypes are sequenced by SNPtagger software to select htSNPs, a minimum marker to easily determine CYP2D6 genotypes mainly found in Koreans. Examples of the htSNP combinations selected according to the present invention are shown in FIGS. 34 to 39.

The htSNP combination selected according to the present invention may be used to determine a genotype of a human CYP2D6 gene. Thus, the present invention provides a method of determining a genotype of a human CYP2D6 gene. The method includes following steps:

(a) collecting a biological sample from humans;

(b) extracting nucleic acid from the sample collected at operation (a);

(c) performing PCR with a primer which amplifies a human CYP2D6 gene or a fragment thereof by using the nucleic acid extracted at operation (b) as a template;

(d) determining presence of at least 11 variants in a CYP2D6 gene including one from −1426C>T, 100C>T and 1039C>T; one from −1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 2988G>A; 4125-4133insGTGCCCACT; 2D6 deletion; and 2D6 duplication in the genetic sequence of the PCR products obtained at operation (c).

The method of extracting the nucleic acid at operation (b) is the same as described above.

As described above, the fragment of the human CYP2D6 gene refers to a fragment which includes known variants of a human CYP2D6 gene, e.g. single nucleotide polymorphism (SNP). The primer which may be used in operation (c) may include genetic sequences selected from references 106 and 107, references 121 to 127, references 129 to 136, references 138, 139, 149 and 150.

The variants at operation (d) may be selected from the htSNPs in FIGS. 34 to 39. For example, as shown in FIG. 34, the presence of the variants including one from −1426C>T, 100C>T and 1039C>T; one from −1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 2988G>A; 4125-4133insGTGCCCACT; 2D6 deletion; and 2D6 duplication may be determined.

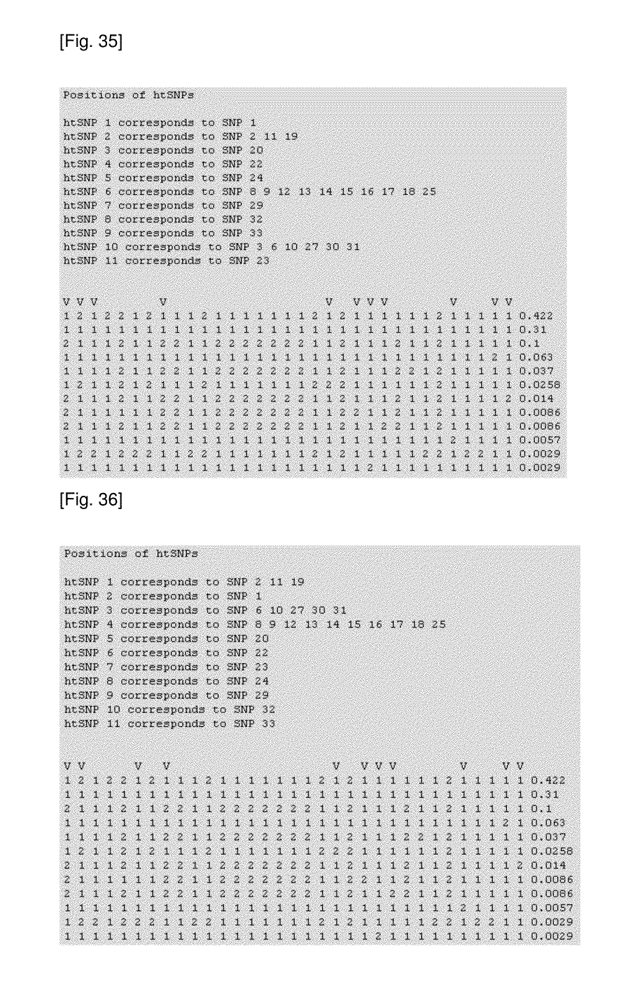

As shown in FIG. 35, the presence of the variants including −1584C>G; one selected from −1426C>T, 100C>T and 1039C>T; 1611T>A; 1758G>A; 2573insC; one from −740C>T, −678G>A, 214G>C, 221C>A, 223C>G, 227T>C, 232G>C, 233A>C, 245A>G and 2850C>; one from −1245insGA, −1028T>C, −377A>C, 3877G>A, 4388C>T and 4401C>T; 4125-4133insGTGCCCACT; 2D6 depletion; and 2D6 duplication may be determined.

Further, as shown in FIG. 36, the presence of the variants including one from −1426C>T, 100C>T and 1039C>T; −1584C>G; one from −1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 4125-4133insGTGCCCACT; 2D6 depletion; and 2D6 duplication may be determined.

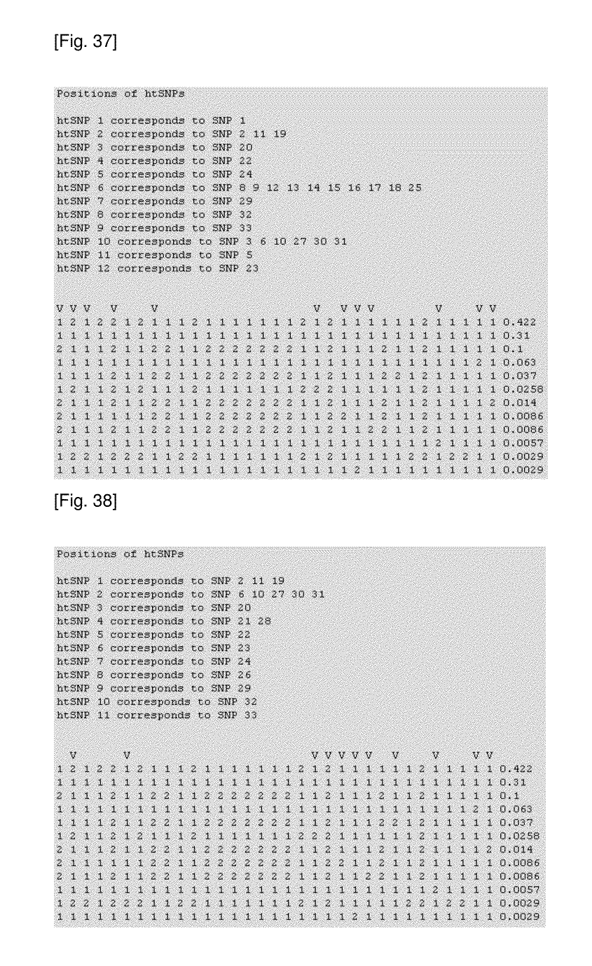

Further, as shown in FIG. 37, the presence of the variants including −1584C>G; one from −1426C>T, 100C>T and 1039C>T; 1611T>A; 1758G>A; 2573insC; one selected from −740C>T, −678G>A, 214G>C, 221C>A, 223C>G, 227T>C, 232G>C, 233A>C, 245A>G and 2850C>T; one from −1245insGA, −1028T>C, −377A>G, 3877G>A, 4388C>T and 4401C>T; 4125-4133insGTGCCCACT; −1235A>G; 1887insTA; 2D6 depletion; and 2D6 duplication may be determined.

Further, as shown in FIG. 38, the presence of the variants including one from −1426C>T, 100C>T and 1039C>T; one from −1028T>C, −377A>G, 3877G>A, 4388C>T and 4401C>T; 1611T>A; one from 1661G>C and 4180G>C; 1758G>A; 1887insTA; 2573insC; 2988G>A; 4125-4133insGTGCCCACT; −1235A>G; 1887insTA; 2D6 depletion; and 2D6 duplication may be determined.

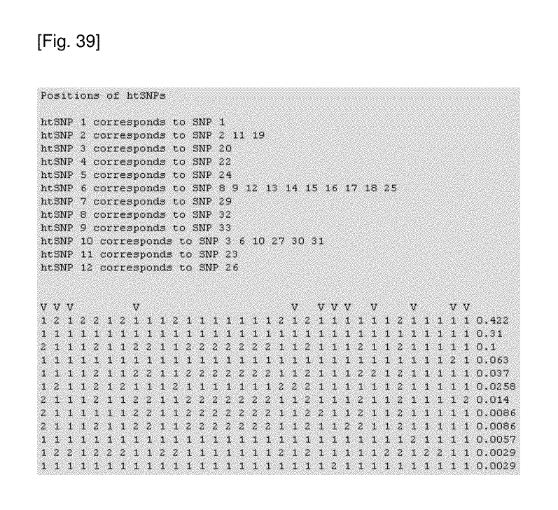

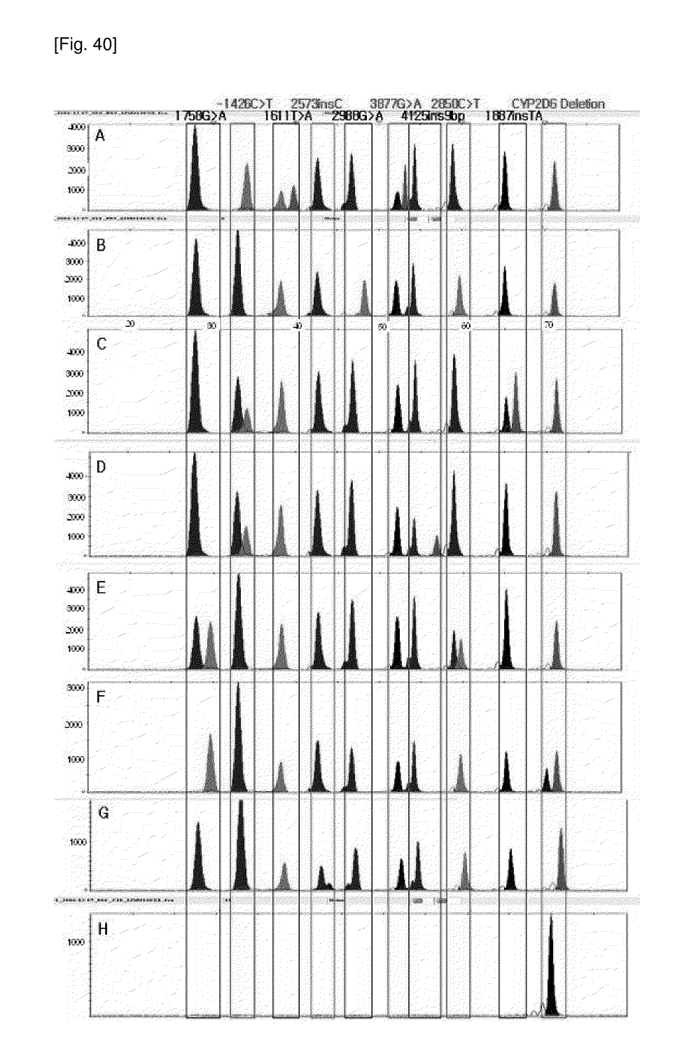

Further, as shown in FIG. 39, the presence of the variants including −1584C>G; one from −1426C>T, 100C>T and 1039C>T; 1611T>A; 1758G>A; 2573insC; one from −740C>T, −678G>A, 214G>C, 221C>A, 223C>G, 227T>C, 232G>C, 233A>C, 245A>G and 2850C>T; one from −1245insGA, −1028T>C, −377A>G, 3877G>A, 4388C>T and 4401C>T; 1887insTA; 2988G>A; 4125-4133insGTGCCCACT; 2D6 depletion; and 2D6 duplication may be determined.

Preferably, the presence of the variants in FIG. 34 may be determined. The variants which are determined at operation (d) are based on variants in the CYP2D6 gene mainly found in Koreans. Thus, it is very specific to determine a haplotype and a genotype of the CYP2D6 gene of Koreans.

The variants in the CYP2D6 gene in the genetic sequence of the PCR products at operation (d) may be determined by using polymorphism analysis methods known in the art. Preferably, the SNaPshot analysis (refer to [Peter M. Vallone, et al., Int J Legal Med, 2004, 118:147-157]), electrophoretic analysis, or a combination thereof may be employed to determine the variants. If the variant in the CYP2D6 includes a SNP, the SNaPshot analysis may be employed.

The SNaPshot analysis refers to a method of determining a genotype through PCR reaction with a primer having an annealed genetic sequence (excluding SNP region) around a SNP position and ddNTP. The SNaPshot analysis which is used in the present invention is designed and manufactured by a known method based on the SNP of the CYP2D6 gene determined at operation (c). The SNaPshot used may vary as long as it has a base right next to the SNP position as 3′end, includes an annealed genetic sequence adjacent to the SNP position and has a T base added to 5′end. Preferably, the annealed genetic sequence adjacent to the SNP position is approximately 20 bp long. If several SNPs are determined simultaneously, the length of a T base at the 5′end of the SNaPshot primers is designed to vary. For example, five T bases are added to the 5′ end so that the primers differ in size, thereby varying the length of the PCR products. Then, the SNaPshot primers are coupled with ddNTP complementary to each SNP. Those composites differ in size depending on the SNP. Thus, several SNPs can be determined simultaneously.

For example, the primer which has a genetic sequence selected from references 141 to 148, and references 152 and 153 may be used to investigate the htSNP combination in FIG. 34 at operation (c). More preferably, all primers which have genetic sequences selected from references 141 to 148, and references 152 and 153 may be used. Then, the genetic sequence of the PCR products that are amplified by the SNaPshot analysis may be analyzed by known genetic sequencing methods. The genetic sequencing methods may vary as long as they are known in the art, and preferably include an automated DNA sequencing.

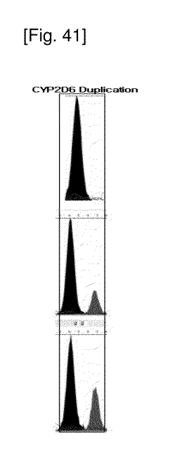

According to another exemplary embodiment of the present invention, availability of the htSNP combinations selected according to the present invention is confirmed. After the SNaPshot analysis is performed by using the htSNP combination in FIG. 34, the genetic sequence of the obtained PCR products is analyzed. As a result, the method according to the present invention has been confirmed to simultaneously determine the CYP2D6 genotypes found in Koreans at high speed (refer to FIGS. 40 and 41).

The genotypes of the CYP2D6 gene which can be determined by the method according to the present invention include CYP2D6*1A, CYP2D6*2A, CYP2D6*5, CYP2D6*2N, CYP2D6*10B, CYP2D6*14B, CYP2D6*18, CYP2D6*21B, CYP2D6*41A, CYP2D6*49, CYP2D6*52 and CYP2D6*60. Each of the genotypes and variants corresponding thereto are shown in Table 34. Referring to Table 34, for example, the CYP2D6*1A genotype includes a wild type, and the CYP2D6*2A genotype includes variants in SNP 1, SNP 5, SNP 8, SNP 9, SNP 12-SNP 18, SNP 21, SNP 25 and SNP 28 positions in the genetic sequence of the wild type CYP2D6 gene. The CYP2D6*5 genotype includes 2D5 deletion variant. As the CYP2D6 gene is completely deleted from human chromosomes, enzymes are not produced at all. The CYP2D6*2N genotype includes 2D6 duplication variant. That is, at least two CYP2D6 genes are present in the same chromosome.

The present invention provides a method of determining a genotype of a human CYP2D6 gene by using a gene chip. The method includes following steps:

(a) extracting a gene to be investigated, performing multiplex PCR to the gene and obtaining PCR products including a circumference of aSNP;

(b) performing ASPE reaction by using an ASPE (allele specific primer extension) primer which identifies a specific base of allele;

(c) mixing the reaction product to a gene chip; and

(d) analyzing the gene chip.

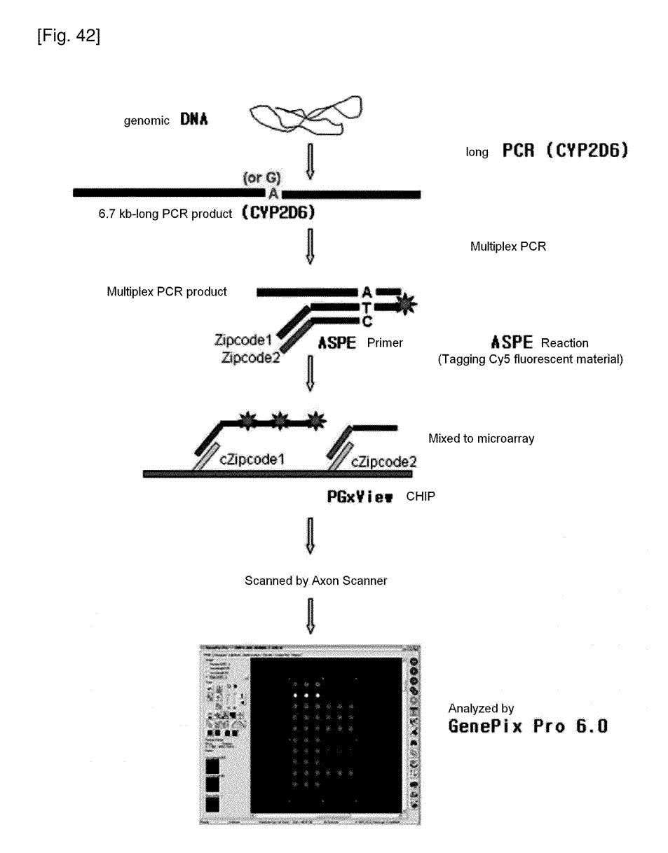

The present invention provides a genotype analysis chip which has a Zip Code oligonucleotide-based chip for determining SNPs (refer to FIG. 42).

A pair of primers is manufactured for each of SNPs to perform ASPE reaction at operation (b). The ASPE primer is manufactured as a genetic sequence which includes a SNP site at 3′end and is specifically coupled with an allele. The ASPE primer includes Zip Code, i.e. oligonucleotide with 24 bp toward 5′. The Zip Code is manufactured to have different genetic sequences in each allele.

The present invention selected the optimal Zip Code sequence which does not have crossing-over reaction to other samples through experimental verification, among genetic sequences disclosed by papers and genetic sequences designed by bioinformatics technology. Tm of the selected sequence is 61° C. The Zip codes are manufactured not to interrupt each other. The selected genetic sequences have a secondary structure of a hair pin whose ΔG value is −2 and above.

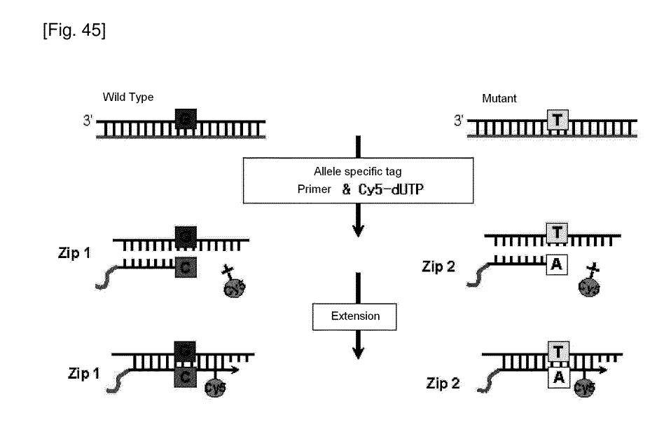

If the ASPE reaction is performed by using the ASPE primers, samples having allele corresponding to the 3′end of the primers react to the primers to generate allele specific extension reaction. If dUTP (Cy5-dUTP) which covalent-bonds with Cyanine 5 (Cy5), fluorescent material, is used to perform the extension reaction, only samples having respective allele label Cy5 fluorescent material (refer to FIG. 45). The fluorescent material is not limited to Cy5, and may employ other materials such as Cy3, TAMRA, TexasRed, Cy3.5, Rhodamin 6G, SyBR Green, etc.

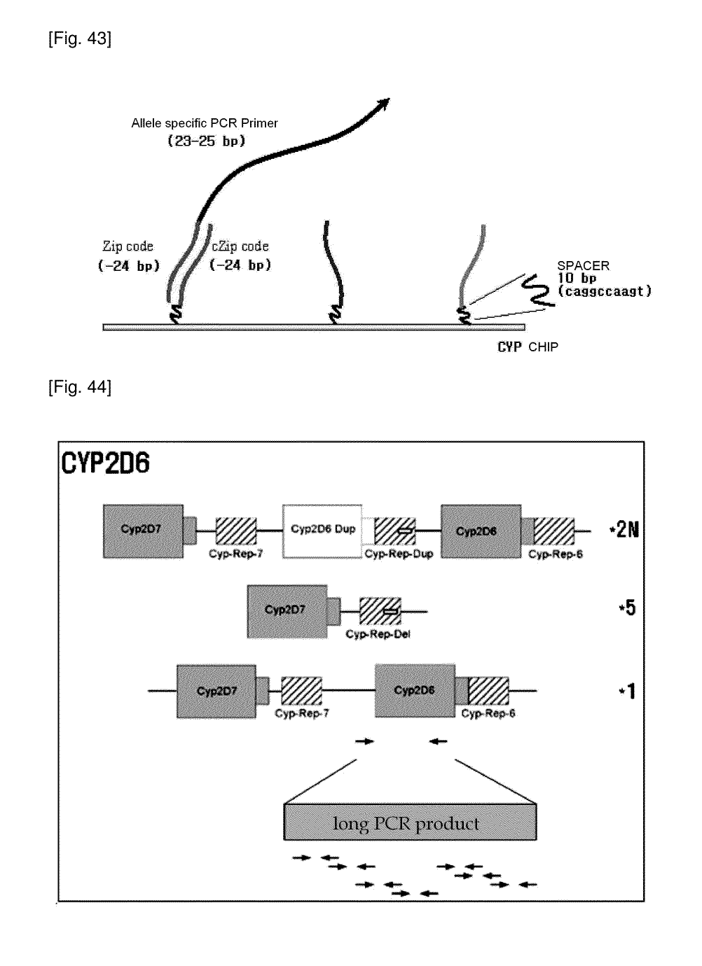

Oligonucleotide probe (cZip Code) which is complementarily coupled with the Zip Code is provided on the analysis chip of the present invention. Thus, each allele included in the samples extended with the Zip Code primers may be identified (refer to FIG. 43).

In the probe, genetic sequences having 10 bp are inserted to 3′ as a spacer to induce hybridization with targets. For example, the spacer sequence is preferably 5′-CAG GCC AAGT-3′.

The probe according to the present invention preferably includes genetic sequences with references 158 to 184.

The method of mixing the reaction product with the gene chip and analyzing the mixed chip at operations (c) and (d) may include a method known in the art. A DNA chip scanner used may vary. More preferably, GenePix 4100B scanner which is manufactured by Axon is used. The scanned images may be analyzed by GenePix Pro 6.0 software.

If the variants in the CYP2D6 gene are analyzed by the gene chip according to the present invention, the results are the same as those confirmed by the sequence analysis. Thus, the gene chip according to the present invention may be cost-effective to analyze the variants of various genes.

4. PXR

The method according to the present invention determines functional variants in a PXR gene by using htSNPs selected based on variants in a PXR gene of Koreans.

The method of selecting htSNPs of a human PXR gene according to the present invention includes following steps:

(a) collecting a biological sample from humans;

(b) extracting nucleic acid from the sample collected at operation (a);

(c) performing PCR with a primer which amplifies a human PXR gene or a fragment thereof by using the nucleic acid extracted at operation (b) as a template;

(d) determining presence of variants by sequencing a PCR product obtained at operation (c);

(e) determining a haplotype in the genetic sequence of the PCR products that is confirmed to have the variants in operation (d); and

(f) sequencing the haplotype determined at operation (e) with SNPtagger software and selecting htSNPs.

The method of extracting the nucleic acid from the sample collected at operation (a) is not limited, and is known in the art. Alternatively, extraction kits may be used to extract the nucleic acid. For example, DNA or RNA extraction kits which are manufactured by Qiagen (USA) and Stratagene (USA) may be used. If RNAs are extracted, cDNA is manufactured by reverse transcription to be used.

The fragment of the human PXR gene at operation (c) refers to a fragment which includes known variants in a human PXR gene, e.g. single nucleotide polymorphism (SNP). The primer which amplifies the human PXR gene or the fragment thereof may be designed based on a genetic sequence of a human PXR gene or a fragment thereof, and may be selected from primers with references 221 to 240, but not limited thereto.

The variants at operation (d) include SNP, gene deletion and gene duplication, but not limited thereto. For example, the variants may include 22 variants as in Table 48.

The method of determining the presence of the variants at operation (d) may include a variant detecting method which is known in the art. Preferably, genetic sequencing, electrophoretic analysis, and RFLP analysis may be performed to determine the presence of the variants. The genetic sequencing may be performed by an automated DNA sequencer or by pyrosequencing.

The presence of the variants at operation (d) may be determined by comparing genetic sequences of a wild type PXR gene. The genetic sequence of the wild type PXR gene, e.g. genetic sequences of reference 2200 (GenBank accession No.: NT_{—}005612) or each genetic sequence of PXR genotypes known in the art may be used. Also, cut phases of a restriction enzyme of the wild type PXR gene may be compared through RFLP analysis. The deletion or duplication of the PXR gene may be determined by electrophoretic analysis of PCR products.

The frequencies and types of haplotypes in the genetic sequence of the PCR products that are confirmed to have variants at operation (d) may be analyzed by using a technical program known in the art or a program sold in the market. For example, Haploview which is distributed free of charge, or SNPAlyze which is a commercialized program may be used. The Haploview software are known in the art, and more preferably downloaded from http://www.broad.mit.edu/mpg/haploview.

The method according to the present invention may additionally include repetition of operations (a) to (e). To determine variant phases of a PXR gene and haplotypes thereof within a particular group such as races or patients, operation (f) may be performed after frequencies of PXR genotypes are examined and frequent PXR genotypes are selected from the group.

At operation (f), the htSNPs are selected by sequencing the haplotypes determined at operation (e) with SNPtagger software. The SNPtagger software are known in the art, e.g. Genehunter, Merlin, Allegro, SNPHAP, htSNP finder (PCA based), and more preferably downloaded from http://www.well.ox.ac.uk/˜xiayi/haplotype or http://slack.ser.man.ac.uk/progs/htsnp.html.

The selected htSNPs may be verified to improve accuracy to thereby determine diplotypes. As a genotype of humans is determined by double-stranded chromosomes, the genotype is decoded to determine two haplotype combinations. If several SNP are determined simultaneously, a combination of a particular haplotype may be the same as that of another haplotype. If the genotype is decoded with the diagnosis developed according to the present invention, it should be verified whether to determine the genotype accurately. Such verification may be performed by analyzing whether the genotype is decoded from the gene analysis result, using Matlab (The math Works Inc., US).

According to an exemplary embodiment of the present invention, variants in the PXR gene of Koreans are investigated first to select htSNPs, functional variants of the PXR gene of Koreans. As a result, a total of 22 SNPs were found in the PXR gene of Koreans (refer to Table 48).

According to another exemplary embodiment of the present invention, a haplotype of six functional variants among the 22 selected SNPs is determined by SNPAlyze program manufactured by DYNACOM to determine a total of 14 haplotypes (refer to Table 49).

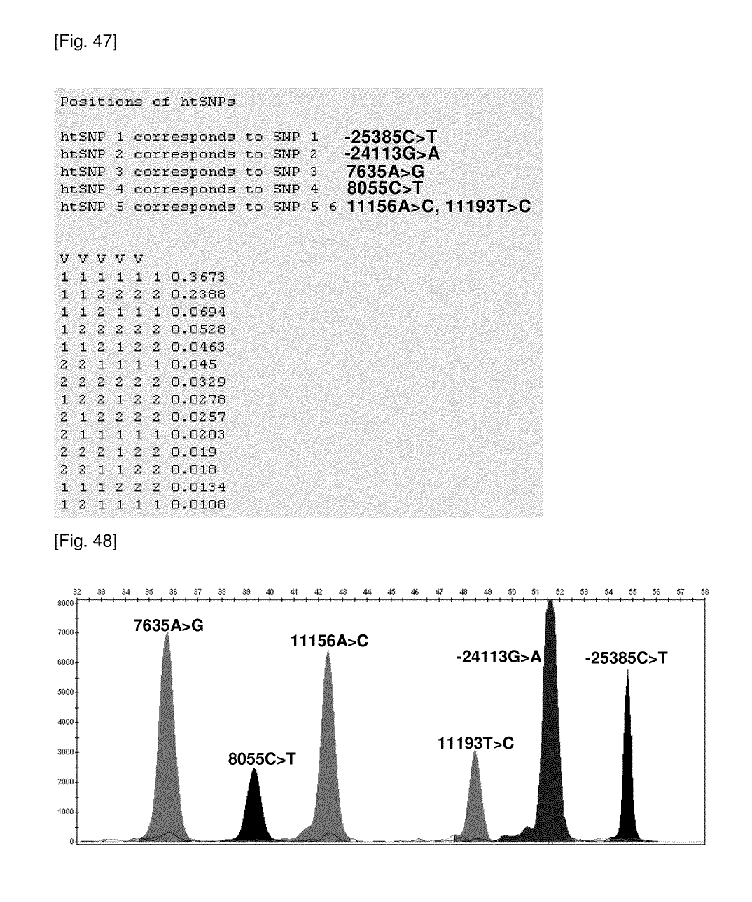

According to another exemplary embodiment of the present invention, the 14 haplotypes are sequenced with SNPtagger software to select htSNPs, a minimum marker which easily determines functional variants in the PXR gene found in Koreans (refer to FIG. 47).

The htSNP combination selected according to the present invention may be used to determine the functional variants of a human PXR gene. Thus, the present invention provides a method of determining functional variants of a human PXR gene. The method includes following steps:

(a) collecting a biological sample from humans;

(b) extracting nucleic acid from the sample collected at operation (a);

(c) performing PCR with a primer which amplifies a human PXR gene or a fragment thereof by using the nucleic acid extracted at operation (b) as a template;

(d) determining a presence of functional variants in the PXR gene selected from −25385C>T, −24113G>A, 7635A>G, 8055C>T, 11156A>C and 11193T>C in the genetic sequence of the PCR products obtained at operation (c).

The method of extracting the nucleic acid from the sample collected at operation (b) is the same as that described above.

The fragment of the human PXR gene refers to a fragment which includes known variants in a human PXR gene, e.g. single nucleotide polymorphism (SNP). The primer which can be used at operation (c) may be selected primers with references 242 to 247, but not limited thereto.

The SNPs which are investigated at operation (d) are based on the functional variants of the PXR gene found in Koreans, and are very specific to determine the haplotype of the functional variants and the functional variants of the PXR gene of Koreans.

The presence of the variants in the PXR gene in the genetic sequence of the PCR products at operation (d) may be determined by polymorphism analysis methods known in the art. Preferably, the presence of the variants may be determined by SNaPShot analysis (refer to [Peter M. Vallone, et al., Int J Legal Med, 2004, 118:147-157]), electrophoretic analysis or a combination thereof, and more preferably by SNaPshot analysis.

The SnaPShot analysis refers to a method of determining a genotype through PCR reaction with a primer having an annealed genetic sequence (excluding SNP region) around a SNP position and ddNTP. The SNaPshot analysis which is used in the present invention is designed and manufactured by a known method based on the SNP of the PXR gene investigated at operation (d). The SNaPshot used may vary as long as it has a base right next to the SNP position as 3′end, includes an annealed genetic sequence adjacent to the SNP position and has a T base added to 5′end. More preferably, a primer may be selected from primers with references 242 to 2457. Preferably, the annealed genetic sequence adjacent to the SNP position is approximately 20 bp long. If several SNP are determined simultaneously, the length of the T base at 5′end of the SNaPshot primers is designed to vary. For example, five T bases are added to 5′ end so that the primers differ in size, thereby varying the length of the PCR products. Then, the SNaPshot primers are coupled with ddNTP complementary to each SNP. Those composites differ in size depending on the SNP. Thus, several SNPs can be determined simultaneously.

To determine whether the genotyping results using the SNaPshot analysis are correct, another genotyping method is performed. Another genotyping method is not limited, and preferably includes an automated DNA sequencing or pyrosequencing.

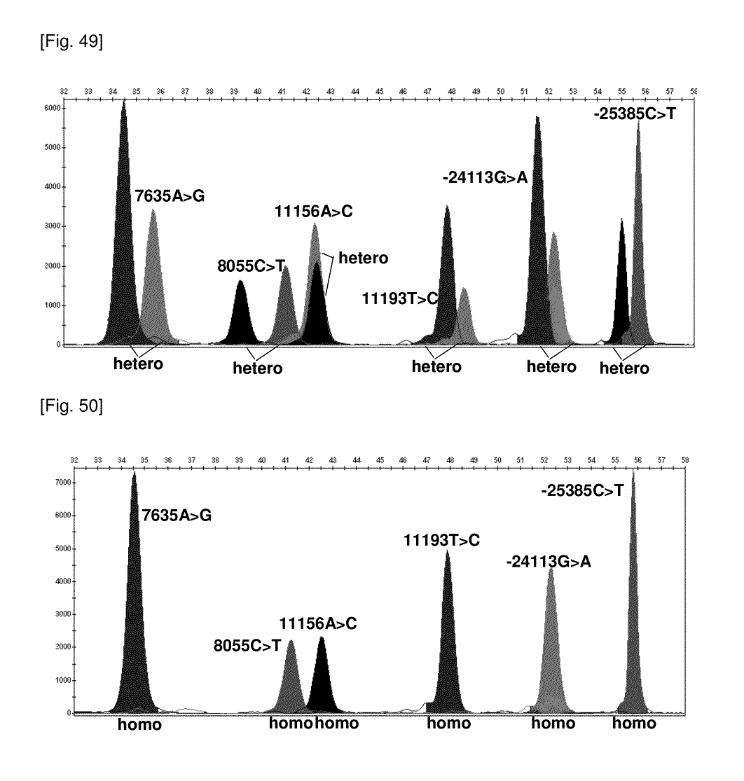

According to another exemplary embodiment of the present invention, availability of the htSNP combinations selected according to the present invention was confirmed. The SNaPshot analysis is performed by using the htSNP combination in FIG. 47, and genetic sequences of the obtained PCR products are analyzed. As a result, the method according to the present invention was confirmed to simultaneously determine the functional variants in the PXR gene found in Koreans, at high speed (refer to FIGS. 48 to 50).

The functional variants in the PXR gene which can be determined by the method according to the present invention include −25385C>T, −24113G>A, 7635A>G, 8055C>T, 11156A>C and 11193T>C.

5. UGT1A

A method of determining functional variants in human UGT1A genes according to the present invention includes following steps:

(a) collecting a biological sample from humans;

(b) extracting nucleic acid from the sample collected at operation (a);

(c) individually amplifying human UGT1A genes by using the nucleic acid extracted at operation (b); and

(d) sequencing the genes amplified at operation (c) and determining presence of functional variants of UGT1A genes selected from −39(TA)6>(TA)7, 211G>A, 233C>T and 686C>A in a UGT1A1 gene; 31T>C, 133C>T and 140T>C in a UGT1A3 gene; 31C>T, 142T>G and 292C>T in a UGT1A4 gene; 19T>G, 541A>G and 552A>C in a UGT1A6 gene; 387T>G, 391C>A, 392G<A, 622T>C and 701T>C in a UGT1A7 gene; and −118T9>T10, 726T>G and 766G>A in a UGT1A9 gene.

The method of determining polymorphisms of UGT1A genes related to sensitivity to irinotecan according to the present invention includes following steps;

(a) collecting a biological sample from humans;

(b) extracting nucleic acid from the sample collected at operation (a);

(c) individually amplifying human UGT1A genes by using the nucleic acid extracted at operation (b); and

(d) sequencing the genes amplified at operation (c) and determining presence of UGT1A genetic variants selected from 211G>A, 233C>T and 686C>A in a UGT1A1 gene; 19T>G, 541A>G and 552A>C in a UGT1A6 gene; and −118T9>T10, 726T>G and 766G>A in a UGT1A9 gene.

The method according to the present invention employs an optimal polymorphism tagging set which is selected based on polymorphism of UGT1A genes mainly found in Koreans, and determines functional variants in UGT1A genes or drug sensitivity. The method according to the present invention is cost and time-effective to analyze the UGT1A genes of Koreans, compared with existing methods.

At operation (a) according to the present invention, the biological sample is collected from humans, preferably Asians including Koreans, Chinese and Japanese, and more preferably Koreans. The biological sample may include blood, skin cells, mucous cells or hair, and more preferably blood.

At operation (b) according to the present invention, the nucleic acid is extracted from the biological sample collected at operation (a). The nucleic acid may include DNA or RNA, preferably DNA, and more preferably genomic DNA. The process of extracting the nucleic acid from the collected sample is not limited, and may be performed according to skills known in the art. Alternatively, DNA or RNA extraction kits, e.g. kits manufactured by Quiagen (USA) of Stratagene (USA) may be used.

At operation (c) according to the present invention, the UGT1A genes are amplified with primers by using the nucleic acid extracted at operation (b) as a template. If the nucleic acid extracted at operation (b) is RNA, it is converted into cDNA by reverse transcription to be used as a template. The primers are designed and manufactured by a known method, based on genetic sequences of human UGT1A genes or a fragment thereof.

At operation (c) according to the present invention, UGT1A1, UGT1A3, UGT1A4, UGT1A6, UGT1A7 and UGT1A9 genes are preferably amplified to determine the functional variants in the UGT1A genes. Preferably, UGT1A1, UGT1A6 and UGT1A9 genes are amplified to determine polymorphisms of UGT1A genes determining sensitivity to irinotecan.

At operation (d) according to the present invention, the functional variants or polymorphism related to drug sensitivity of the UGT1A genes are analyzed by using the UGT1A genes amplified at operation (c). Polymorphism analysis methods which are known in the art may be used to analyze the functional variants or polymorphism. For example, SNaPshot analysis, electrophoretic analysis, pyrosequencing or a combination thereof may be performed.

More specifically, if the variants in the UGT1A gene to be analyzed include SNPs, the SNaPshot analysis is preferable. In the SNaPshot analysis, primers and ddNTP which can anneal a region adjacent to the SNP positions are used to perform PCR reaction. The primers which are used in the SNaPshot analysis are designed and manufactured by known methods based on SNPs of UGT1A genes. For example, the primers are designed and manufactured so that a base right next to the SNP position is 3′end, includes an annealed genetic sequence adjacent to the SNP position and has a T base added to 5′end. Preferably, the annealed genetic sequence is approximately 20 bp long. If several SNP are determined simultaneously, the length of a T base at the 5′end of the SNaPshot primers is designed to differ, thereby varying the length of the PCR products.

Primers which have genetic sequences with references from 2905 to 314 may be used to perform the SNaPshot analysis determining the functional variants in UGT1A genes. Primers which have genetic sequences with references from 315 to 322 may be used to perform the SNaPshot analysis determining polymorphism related to sensitivity to irinotecan of UGT1A genes.

The genetic sequences of the PCR products which are amplified by the SNaPshot analysis may be analyzed by known sequencing methods. Preferably, the genetic sequences of the PCR products may be analyzed by automated sequencing methods, but not limited thereto.

If the variants of the UGT1A genes to be analyzed are not SNPs (e.g. −39(TA)6>(TA)7 in a UGT1A1 gene), known pyrosequencing may be performed instead of the SNaPshot analysis. The pyrosequencing estimates expression of PPi (inorganic pyrophosphate) discharged while DNA is polymerized. According to an exemplary embodiment of the present invention, primers which have genetic sequences with references 292 to 294 may be used to perform pyrosequencing determining −39(TA)6>(TA)7 of a UGT1A1 gene.

Hereinafter, exemplary embodiments of the present invention will be described in detail. The following exemplary embodiments exemplify the present invention, and the present invention is not limited to the following exemplary embodiments.

<CYP1A2>

### Exemplary Embodiment 1

### Determining Genotype of CYP1A2 Gene in Koreans

<1-1> Amplification of CYP1A2 Gene

After blood was collected from 48 healthy subjects, DNA was separated from blood by using a genomic DNA separating kit manufactured by Qiagen. The CYP1A2 gene includes seven exons, and is approximately 11 kb long. The CYP1A2 gene was divided into 15 fragments to perform PCR. Primers which are used in each PCR are shown in Table 1. A, T, G and C in genetic sequences written in the present specification refer to adenine, thymine, guanine and cytosine.

TABLE 1
primers for amplifying CYP1A2 gene and genetic sequences thereof

| PCR | | | |
|---|---|---|---|
| products | Primer name | Genetic sequences (5′→3′) | References |
| CYP1A2p7 | CYP1A2p7_F | gctacacatgatcgagctatac | 2 |
| | CYP1A2p7_R | caggtctcttcactgtaaagtta | 3 |
| CYP1A2p6 | CYP1A2p6_F | caggaaacagctatgaccttgtcatgccccagcttc | 4 |
| | CYP1A2p6_R | tgtaaaacgacggccagtccactattggaatgtgcctga | 5 |
| CYP1A2p5 | CYP1A2p5_F | caggaaacagctatgacctccaaggtcttcccacca | 6 |
| | CYP1A2p5_R | tgtaaaacgacggccagtcccaagcaatccttctgc | 7 |
| CYP1A2p4 | CYP1A2p4_F | caggaaacagctatgaccgcacagtggctcacacct | 8 |
| | CYP1A2p4_R | tgtaaaacgacggccagttcaaaggtttatccttgcttga | 9 |
| CYP1A2p3 | CYP1A2p3_F | caggaaacagctatgacctcctcacgtaagtccatgaatatc | 10 |
| | CYP1A2p3_R | tgtaaaacgacggccagtccccacaacctccttttg | 11 |
| CYP1A2p2 | CYP1A2p2_F | caggaaacagctatgaccccatctcggcctctcaaa | 12 |
| | CYP1A2p2_R | tgtaaaacgacggccagtctaggccaaccaggctca | 13 |
| CYP1A2p1e1a | CYP1A2p1e1a_F | caggaaacagctatgaccggttttgcaggttgttgga | 14 |
| | CYP1A2p1e1a_R | tgtaaaacgacggccagtaggctccccgtctttctg | 15 |
| CYP1A2p1e1b | CYP1A2p1e1b_F | gccaagagttgatccttcca | 16 |
| | CYP1A2p1e1b_R | gctggctctctcctccaca | 17 |
| CYP1A2e2a | CYP1A2e2a_F | caggaaacagctatgaccggagagagccagcgttca | 18 |
| | CYP1A2e2a_R | tgtaaaacgacggccagtccacaccggtccagagtc | 19 |
| CYP1A2e2b | CYP1A2e2b_F | caggaaacagctatgacccagggcgacgatttcaag | 20 |
| | CYP1A2e2b_R | tgtaaaacgacggccagttcctaggccttggcaaca | 21 |
| CYP1A2e3 | CYP1A2e3_F | caggaaacagctatgacctcacgttgcttccctgtg | 22 |
| | CYP1A2e3_R | tgtaaaacgacggccagtgcatagcccaggctcaaa | 23 |
| CYP1A2e4 | CYP1A2e4_F | caggaaacagctatgacctttgagcctgggctatgc | 24 |
| | CYP1A2e4_R | tgtaaaacgacggccagtccctaactgccccatgaa | 25 |
| CYP1A2e5 | CYP1A2e5_F | caggaaacagctatgaccgtgcctgctgtgtgcaag | 26 |
| | CYP1A2e5_R | tgtaaaacgacggccagttggaggccaatagggtca | 27 |
| CYP1A2e6 | CYP1A2e6_F | caggaaacagctatgaccccaggcgcaaagagaagt | 28 |
| | CYP1A2e6_R | tgtaaaacgacggccagtataggcgcaccaccatgt | 29 |
| CYP1A2e7 | CYP1A2e7_F | cttcccacctacccttcatt | 30 |
| | CYP1A2e7_R | tggggtcttgctctgtcact | 31 |

Positions of the primers and sizes of the PCR products are shown in Table 2. Positions of nucleotide are written according to naming method of Cytochrome P450 (CYP) Allele Nomenclature Committee (http://www.cypalleles.ki.se/cyp1a2.htm).

TABLE 2
Positions of primers and sizes of PCR products

| | | | | Size of PCR |
|---|---|---|---|---|
| PCR products | Primer name | References | Positions | products |
| CYP1A2p7 | CYP1A2p7_F | 2 | −3994?−3972 | 597 |
| | CYP1A2p7_R | 3 | −3420?−3397 | |
| CYP1A2p6 | CYP1A2p6_F | 4 | −3848?−3830 | 668 |
| | CYP1A2p6_R | 5 | −3237?−3216 | |
| CYP1A2p5 | CYP1A2p5_F | 6 | −3297?−3276 | 671 |
| | CYP1A2p5_R | 7 | −2667?−2659 | |
| CYP1A2p4 | CYP1A2p4_F | 8 | −2772?−2704 | 673 |
| | CYP1A2p4_R | 9 | −2107?−2085 | |
| CY1A2p3 | CYP1A2p3_F | 10 | −2298?−2274 | 631 |
| | CYP1A2p3_F | 11 | −1721?−1702 | |
| CYP1A2p2 | CYP1A2p2_F | 12 | −1807?−1788 | 586 |
| | CYP1A2p2_R | 13 | −1274?−1252 | |
| CYP1A2p1e1a | CYP1A2p1e1a_F | 14 | IVS1 − 434?IVS1 − 415 | 611 |
| | CYP1A2p1e1a_R | 15 | IVS1 + 68?IVS + 86 | |
| CYP1A2p1e1b | CYP1A2p1e1b_F | 16 | IVS1 − 119?IVS − 99 | 758 |
| | CYP1A2p1e1b_F | 17 | IVS2 − 247?IVS2 − 228 | |
| CYP1A2e2a | CYP1A2e2a_F | 18 | IVS2 − 241?IVS2 − 223 | 685 |
| | CYP1A2e2a_R | 19 | Exon2 + 390?Exon2 + 48 | |
| CYP1A2e2b | CYP1A2e2b_F | 20 | Exon2 + 309?Exon2 + 327 | 674 |
| | CYP1A2e2b_R | 21 | IVS2 + 90?IVS2 + 108 | |
| CYP1A2e3 | CYP1A2e3_F | 22 | IVS3 − 277?IVS3 − 259 | 592 |
| | CYP1A2e3_R | 23 | IVS3 + 140?IVS3 + 158 | |
| CYP1A2e4 | CYP1A2e4_F | 24 | IVS4 − 331?IVS4 − 313 | 673 |
| | CYP1A2e4_R | 25 | IVS + 214?IVS4 + 232 | |
| CYP1A2e5 | CYP1A2e5_F | 26 | IVS5 − 189?IVS − 171 | 642 |
| | CYP1A2e5_R | 27 | IVS5 + 276?IVS5 + 295 | |
| CYP1A2e6 | CYP1A2e6_F | 28 | IVS6 − 219?IVS6 − 201 | 683 |
| | CYP1A2e6_R | 29 | IVS6 + 324?IVS6 + 342 | |
| CYP1A2e7 | CYP1A2e7_F | 30 | IVS7 − 132?IVS7 − 112 | 689 |
| | CYP1A2e7_R | 31 | Exon7 + 536?Exon7 + 556 | |

Reaction conditions with respect to PCR fragments are as shown in Table 3.

TABLE 3
PCR reaction conditions

| PCR products | Reaction conditions |
|---|---|
| CYP1A2p7 | 94° C. 4 min, (94° C. 30 sec, 55° C. 30 sec, 72° C. |
| | 40 sec) 35 cycles, 72° C. 5 min |
| CYP1A2p6 | 94° C. 4 min, (94° C. 30 sec, 60° C. 30 sec, 72° C. |
| | 40 sec) 35 cycles, 72° C. 5 min |
| CYP1A2p5 | 94° C. 4 min, (94° C. 30 sec, 60° C. 30 sec, 72° C. |
| | 40 sec) 35 cycles, 72° C. 5 min |
| CYP1A2p4 | 94° C. 4 min, (94° C. 30 sec, 60° C. 30 sec, 72° C. |
| | 40 sec) 35 cycles, 72° C. 5 min |
| CYP1A2p3 | 94° C. 4 min, (94° C. 30 sec, 60° C. 30 sec, 72° C. |
| | 40 sec) 35 cycles, 72° C. 5 min |
| CYP1A2p2 | 94° C. 4 min, (94° C. 30 sec, 68.5° C. 30 sec, 72° C. |
| | 40 sec) 35 cycles, 72° C. 5 min |
| CYP1A2p1e1a | 94° C. 4 min, (94° C. 30 sec, 60° C. 30 sec, 72° C. |
| | 40 sec) 35 cycles, 72° C. 5 min |
| CYP1A2p1e1b | 94° C. 4 min, (94° C. 30 sec, 60° C. 30 sec, 72° C. |
| | 45 sec) 35 cycles, 72° C. 5 min |
| CYP1A2e2a | 94° C. 4 min, (94° C. 30 sec, 60° C. 30 sec, 72° C. |
| | 40 sec) 35 cycles, 72° C. 5 min |
| CYP1A2e2b | 94° C. 4 min, (94° C. 30 sec, 60° C. 30 sec, 72° C. |
| | 40 sec) 35 cycles, 72° C. 5 min |
| CYP1A2e3 | 94° C. 4 min, (94° C. 30 sec, 60° C. 30 sec, 72° C. |
| | 40 sec) 35 cycles, 72° C. 5 min |
| CYP1A2e4 | 94° C. 4 min, (94° C. 30 sec, 60° C. 30 sec, 72° C. |
| | 40 sec) 35 cycles, 72° C. 5 min |
| CYP1A2e5 | 94° C. 4 min, (94° C. 30 sec, 60° C. 30 sec, 72° C. |
| | 40 sec) 35 cycles, 72° C. 5 min |
| CYP1A2e6 | 94° C. 4 min, (94° C. 30 sec, 60° C. 30 sec, 72° C. |
| | 40 sec) 35 cycles, 72° C.5 min |
| CYP1A2e7 | 94° C. 4 min, (94° C. 30 sec, 58° C. 30 sec, 72° C. |
| | 40 sec) 35 cycles, 72° C. 5 min |

<1-2> Sequencing PCR Products

The PCR products which are obtained according to the exemplary embodiment <1-1> were sequenced by an automated DNA sequencer. The primers used are as shown in Table 4.

TABLE 4
Primers used for sequencing

| PCR product | Primer name | Genetic sequences (5′→3′) | Reference |
|---|---|---|---|
| CYP1A2p7 | CYP1A2p7_F | gctacacatgatcgagctatac | 32 |
| | CYP1A2p7_R | caggtctcttcactgtaaagtta | 33 |
| CYP1A2p6 | CYP1A2p6_F | caggaaacagctatga | 34 |
| | CYP1A2p6_R | tgtaaaacgacggccagt | 35 |
| CYP1A2p5 | CYP1A2p5_F | caggaaacagctatga | 36 |
| | CYP1A2p5_R | tgtaaaacgacggccagt | 37 |
| CYP1A2p4 | CYP1A2p4_F | caggaaacagctatga | 38 |
| | CYP1A2p4_R | tgtaaaacgacggccagt | 39 |
| CYP1A2p3 | CYP1A2p3_F | caggaaacagctatga | 40 |
| | CYP1A2p3_R | tgtaaaacgacggccagt | 41 |
| CYP1A2p2 | CYP1A2p2_F | caggaaacagctatga | 42 |
| | CYP1A2p2_R | tgtaaaacgacggccagt | 43 |
| CYP1A2p1e1a | CYP1A2p1e1a_F | caggaaacagctatga | 44 |
| | CYP1A2p1e1a_R | tgtaaaacgacggccagt | 45 |
| CYP1A2p1e1b | CYP1A2p1e1b_F | gccaagagttgatccttcca | 46 |
| | CYP1A2p1e1b_R | gctggctctctcctccaca | 47 |
| CYP1A2e2a | CYP1A2e2a_F | caggaaacagctatga | 48 |
| | CYP1A2e2a_R | tgtaaaacgacggccagt | 49 |
| CYP1A2e2b | CYP1A2e2b_F | caggaaacagctatga | 50 |
| | CYP1A2e2b_R | tgtaaaacgacggccagt | 51 |
| CYP1A2e3 | CYP1A2e3_F | caggaaacagctatga | 52 |
| | CYP1A2e3_R | tgtaaaacgacggccagt | 53 |
| CYP1A2e4 | CYP1A2e4_F | caggaaacagctatga | 54 |
| | CYP1A2e4_R | tgtaaaacgacggccagt | 55 |
| CYP1A2e5 | CYP1A2e5_F | caggaaacagctatga | 56 |
| | CYP1A2e5_R | tgtaaaacgacggccagt | 57 |
| CYP1A2e6 | CYP1A2e6_F | caggaaacagctatga | 58 |
| | CYP1A2e6_R | tgtaaaacgacggccagt | 59 |
| CYP1A2e7 | CYP1A2e7_F | cttcccacctacccttcatt | 60 |
| | CYP1A2e7_R | tggggtcttgctctgtcact | 61 |

The entire genetic sequences of the CYP1A2 gene which was amplified according to the exemplary embodiment <1-1> were analyzed by an automated DNA sequencer. After being compared with genetic sequences of a wild type CYP1A2 (reference 1), a total of 17 SNPs were found. The results are shown in Table 5. It was determined that SNP −2603insA is novel.

TABLE 5
variants of CYP1A2 gene found in Koreans

| | | rs | Amino acid | Frequency |
|---|---|---|---|---|
| SNP | Naming | number | variants | (%) |
| −3860G > A | *1C | | | 27.08 |
| −3598G > T | | 2069519 | | 9.38 |
| −3594T > G | | 2069520 | | 9.38 |
| −3113G > A | | 2069521 | | 12.50 |
| −2847T > C | | 2069522 | | 11.46 |
| −2808A > C | | 12592480 | | 1.04 |
| −2603insA | | — | | 1.04 |
| −2467de1T | *1D | — | | 43.75 |
| −1708T > C | | 2069525 | | 6.25 |
| −739T > G | *1E | | | 7.29 |
| −163C > A | *1F | 762551 | | 55.21 |
| 1514G > A | *13 | — | G299S | 1.04 |
| 2159G > A | | 2472304 | | 14.58 |
| 2321G > C | | 3743484 | | 9.38 |
| 3613T > C | | 4646427 | | 6.25 |
| 5347C > T | *1B | 2470890 | N516N | 15.63 |
| 5521A > G | | | | 14.58 |

Then, the present inventors performed PCR with the primers having references 38 and 39 and analyzed the genetic sequences of the amplified products with the same method described above, by using DNA of subjects including genetic variants found in the SNPs as a template. The aim was to determine whether the novel SNP is positioned in a single strand of the CYP1A2 gene, whether other variants are present in the same strand, whether the novel SNP is resulted from similar genes positioned in other part of the chromosome.

As a result, it was found that the one SNP is positioned in −2603insA in a promoter. One of the double-stranded DNA was a variant and the other one was wild type (FIG. 1).

### Exemplary Embodiment 2

### Determining Haplotypes of CYP1A2 Variant

The 17 CYP1A2 gene variants found in the exemplary embodiment of the present invention may possibly affect activity of CYP1A2 enzymes depending on combination thereof. The variants of enzyme activity with respect to some haplotypes have already been reported. Thus, the present inventors analyzed the haplotypes due to variants determined in the exemplary embodiment 1, by using SNPAlyze manufactured by DYNACOM. As a result, new haplotypes of Koreans which are not found in other races were found as shown in Table 6.

TABLE 6

| Nucleotide | | | | | | | | | | | |
|---|---|---|---|---|---|---|---|---|---|---|---|
| variant | | | −3860G > A | −3598G > T | −3594T > G | −3113G > A | −2847T > C | −2808A > C | −2603nsA | −2467T > delT | −1708T > C |
| Amino acid | | | | | | | | | | | |
| variant | | | | | | | | | | | |
| Nomenclature | | | *1C | | | | | | | *1D | |
| Haplo- | 1 | *1A | G | G | T | G | T | A | — | T | T |
| type | 2 | *1L | 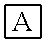 | G | T | G | T | A | — | 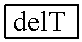 | T |
| | 3 | *1M | G | G | T | G | T | A | — | T | T |
| | 4 | *1N | G | G | 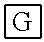 | G | T | A | — | 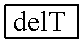 | T |
| | 5 | | G | 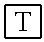 | T | 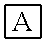 | 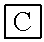 | A | — | 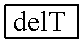 | 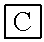 |
| | 6 | | G | G | T | G | T | A | — | T | T |
| | 7 | | 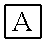 | 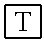 | T | G | T | A | — | 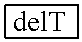 | T |
| | 8 | | G | G | T | G | T | A | — | 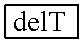 | T |
| | 9 | *1C | 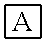 | G | T | G | T | A | — | T | T |
| | 10 | | G | G | 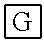 | G | T | A | — | 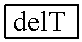 | T |
| | 11 | | G | 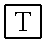 | T | G | 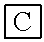 | A | — | 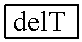 | 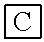 |
| | 12 | | G | 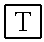 | T | 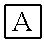 | 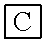 | A | — | 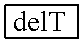 | T |
| | 13 | *1aa | 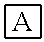 | G | T | G | T | A | — | 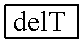 | T |
| | 14 | *1Q | G | G | T | G | T | 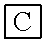 | — | T | T |
| | 15 | | G | 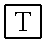 | T | 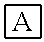 | 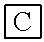 | A | — | 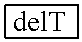 | 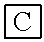 |
| | 16 | | G | 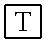 | T | 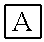 | 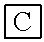 | A | — | 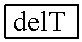 | 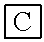 |
| | 17 | | G | G | T | G | T | A | — | T | T |
| Nucleotide | | | | | | | | | | | |
| variant | | | −739T > G | −163C > A | 1514G > A | 2159G > A | 2321G > C | 3613T > C | 5347C > T | 5521A > G | |
| Amino acid | | | | | | | | | | | |
| variant | | | | | G299S | | | | N516N | | |
| Nomenclature | | | *1E | *1F | *13 | | | | *1B | | freq. (%) |
| Haplo- | 1 | *1A | T | C | G | G | G | T | C | A | 40.62 |
| type | 2 | *1L | T | 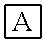 | G | G | G | T | C | A | 22.91 |
| | 3 | *1M | T | 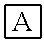 | G | 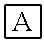 | G | T | 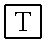 | A | 10.42 |
| | 4 | *1N | T | 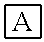 | G | G | 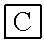 | T | C | 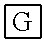 | 8.33 |
| | 5 | | 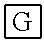 | 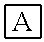 | G | G | G | 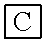 | C | 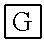 | 3.13 |
| | 6 | | T | C | G | 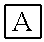 | G | T | C | A | 2.08 |
| | 7 | | T | 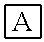 | G | G | G | T | C | A | 2.08 |
| | 8 | | T | 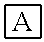 | G | G | G | T | C | A | 1.05 |
| | 9 | *1C | T | C | G | G | G | T | C | A | 1.05 |
| | 10 | | T | 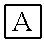 | G | G | 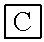 | T | 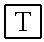 | 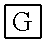 | 1.04 |
| | 11 | | 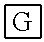 | 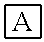 | G | G | G | T | 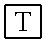 | 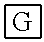 | 1.04 |
| | 12 | | 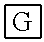 | 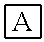 | G | G | G | 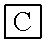 | 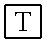 | 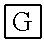 | 1.04 |
| | 13 | *1aa | T | C | G | G | G | T | C | A | 1.04 |
| | 14 | *1Q | T | 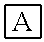 | G | 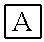 | G | T | 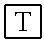 | A | 1.04 |
| | 15 | | 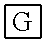 | 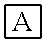 | 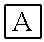 | G | G | 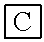 | C | A | 1.04 |
| | 16 | | 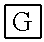 | 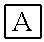 | G | G | G | 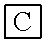 | C | A | 1.04 |
| | 17 | | T | 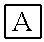 | G | 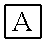 | G | T | 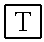 | A | 1.04 |

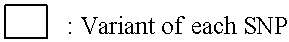

### Exemplary Embodiment 3

### Selection and Verification of htSNPs

It has been reported that several haplotypes, combination of SNPs of the CYP1A2 gene, possibly affect activity of CYP1A2 enzymes. Detailed information on the produced haplotypes can be checked by a minimum marker. The minimum marker is called htSNPs which is required to mark the haplotypes accurately and includes several combinations. The htSNP combinations, an optimal tagging set were selected by SNPtagger software (http://www.well.ox.ac.uk/˜xiayi/haplotype). Examples of the selected htSNP combinations are shown in FIGS. 2 to 6. The selected htSNP combinations are one of optimal tagging sets, in which “1” refers to a wild type, “2” is a variant and ‘V’ means selected htSNPs. The selection of htSNP combinations may vary other than the htSNP combinations in FIGS. 2 to 6.

The found combinations were analyzed by Matlab software (version 7.1, The Math Works Inc., US) to determine diplotypes and genotypes without overlapping each other. The analysis results were used to determine the combinations.

According to the verification results, diplotype and genotypes can be determined without overlapping each other. That means, the htSNP combinations selected according to the present invention are not the same and the analysis for determining the genotypes was not incorrect at all.

### Exemplary Embodiment 4

### Rapid Search of Genetic Variants in CYP1A2 Promoter

Among the 17 SNPs in the CYP1A2 gene found in Koreans and determined in the exemplary embodiment 1, the SNaPshot analysis was performed to search 11 SNPs of promoters affecting activity of CYP1A2 enzymes at high speed. The PCR was performed by using DNA of subjects as a template, and the amplified products were SNaPshot-analyzed. The promoters of the CYP1A2 gene are approximately 4,000 bases, and the primers used for the PCR are as shown in Table 7.

TABLE 7
Primer name and genetic sequences

| PCR product | Primer name | Genetic sequences (5′→3′) | references |
|---|---|---|---|
| CYP1A2_promoter | CYP1A2*1C_F | gctacacatgatcgagctatac | 62 |
| | CYP1A2*1F_R | gggttgagatggagacattc | 63 |

The reaction conditions with respect to the PCR product are as shown in Table 8.

TABLE 8
PCR reaction conditions

| PCR product | Reaction conditions |
|---|---|
| CYP1A2_promoter | 94° C. 1 min, (98° C. 10 sec, 55° C. 30 sec, 68° C. |
| | 4 min) 35 cycles, 72° C. 5 min |

The remaining primers and dNTP which do not react to the amplified PCR product may affect the SNaPshot analysis. To remove the remaining primers and dNTP, 5 μl PCR product was mixed with 2 μl ExoSAP-IT (manufactured by USB) to react at 37° C. for 30 minutes, and then at 80° C. for another 15 minutes to deactivate the remaining enzymes. The product was used to make multiplex SNaPshot reactant by using the primers in Table 9 to perform PCR thereto. The multiplex SNaPshot reactant and the PCR reaction conditions are shown in Tables 10 and 11.

TABLE 9
Primer names and genetic sequences thereof

| Primer names | Genetic sequences (5′→3′) | reference |
|---|---|---|
| −163C/A_F(24) | TTTTAAAGGGTGAGCTCTGTGGGC | 64 |
| −739T/G_F(20) | GCCTGGGCTAGGTGTAGGGG | 65 |
| −2847T/C_F(32) | TTTTTTTTTTTTGCCTTCAAACATGCTCTGTT | 66 |
| −2808A/C_R(36) | TTTTTTTTTTTTTTTTAAAACTGTGGGATCAACCTG | 67 |
| −1708T/C_F(40) | TTTTTTTTTTTTTTTTTTTTAACCATTCAAAAGGAGGTTG | 68 |
| −3860G/A_R(44) | TTTTTTTTTTTTTTTTTTTTTTTTGCATGACAATTGCTTGAATC | 69 |
| −3113G/A_F(48) | TTTTTTTTTTTTTTTTTTTTTTTTTTTTCAAGAGGAATCCAAAG | 70 |
| | AGAC | |
| −2603A7/A8_R2(52) | TTTTTTTTTTTTTTTTTTTTTTTTTTTTTTTTTTATTTTTAAAC | 71 |
| | ATTTTTTT | |
| −3594T/G_R(56) | TTTTTTTTTTTTTTTTTTTTTTTTTTTTTTTTTTTTTTTATTTT | 72 |
| | TAATGTTTTCTT | |
| −3598G/T_F(60) | TTTTTTTTTTTTTTTTTTTTTTTTTTTTTTTTTTTTTTTTCTGT | 73 |
| | AATTTAATTTTTTTAA | |
| −2467delT_F(64) | TTTTTTTTTTTTTTTTTTTTTTTTTTTTTTTTTTTTTTTTTTTT | 74 |
| | TGAGCCATGATTGTGGCACA | |

TABLE 10
Multiplex SNaPshot reactant

| | | | Volume |
|---|---|---|---|
| Composition | | | (/sample) |
| SNaPshot Multiplex Ready Reaction Mix by ABI | | | 2 |
| ½ term buffer solution | | | 3 |
| Enzyme-processed PCR products | | | 3 |
| | Primer names | concentration | |
| Primers | −163C/A_F(24) | 7 mM | 0.1 |
| | −739T/G_F(20) | 3 mM | 0.1 |
| | −2847T/C_F(32) | 5 mM | 0.1 |
| | −2808A/C_R(36) | 5 mM | 0.1 |
| | −1708T/C_F(40) | 20 mM | 0.1 |
| | −3860G/A_R(44) | 20 mM | 0.1 |
| | −3113G/A_F(48) | 7 mM | 0.1 |
| | −2603A7/A8_R2(52) | 100 mM | 0.1 |
| | −3594T/G_R(56) | 70 mM | 0.1 |
| | −3598G/T_F(60) | 100 mM | 0.1 |
| | −2467delT_F(64) | 50 mM | 0.1 |

| | |
|---|---|
| Distilled water | 0.9 |
| Total | 10 |

TABLE 11

| PCR product | Reaction conditions |
|---|---|
| SNaPshot product | (96° C. 10 min, 50° C. 5 sec, 60° C. 30 sec) 30 cycles |

After the reaction was completed, 2 μl SAP (USB) was mixed with 5 μl SNaPshot product to react at 80° C. for 15 minutes, to thereby remove [F]ddNTP. Then, 0.5 μl reactant, 9.25 μl Hi-Di formamide (ABI) and 0.25 μl GeneScan-LIZ size standard material (ABI) were mixed to denature at 95° C. for minutes. Then, the compound was analyzed by 3130XL Genetic Analyzer (ABI). The analysis results are shown in FIGS. 7 to 14.

As shown therein, colors and positions of peaks are displayed differently depending on the variants of the CYP1A2 gene, thereby easily identifying wild types, variants (hetero) having hetero allele and variants (homo) having homo allele. The analysis method according to the present invention is cost and time effective and analyzes the variants of the CYP1A2 gene without difficulty.

<CYP2A6>

### Exemplary Embodiment 5

### Determining Genotype of 2A6 Gene in Koreans

<5-1> Amplification of 2A6 Gene

After blood was collected from 50 healthy subjects, DNA was separated from the blood with a genomic DNA kit manufactured by Qiagen. The CYP2A6 gene includes nine exons, and is approximately 6.9 kb long. The CYP2A6 gene was divided into seven fragments to perform PCR thereto. The primers which are used for the PCR are as shown in Table 12. A, T, G and C in genetic sequences written in the present specification refer to adenine, thymine, guanine and cytosine.

TABLE 12
primers for amplifying CYP2A6 gene and genetic sequences thereof

| PCR products | Primer names | Genetic sequences (5′→3′) | references |
|---|---|---|---|
| CYP2A6_exon1 | exon1F* | GGTCTTCCTCCCCTTCCCAAT | 76 |
| | exon1.R* | CCCAAGATCCTGTCTTTCT | 77 |
| CYP2A6_exon2 | exon2F | TGTGTCCCAAGCTAGGCAGG | 78 |
| | exon2R | GGGAAGACCAGACTGGGGAC | 79 |
| CYP2A6_exon3,4 | exon3, 4F* | CTCTGACTGAGTTTGCAGCTCTG | 80 |
| | exon3, 4R* | GGGACACTGTCTGGAGGGC | 81 |
| CYP2A6_exon5 | exon5F* | GCCCCACTGAAATACCTAAACAAC | 82 |
| | exon5R* | GGGCCTGTGTCATCTGCCT | 83 |
| CYP2A6_exon6 | exon6F* | CCCTCTTTCCACCTTTGGTCTGA | 84 |
| | exon6R* | AAGTCACGTCTCAGGGTCCC | 85 |
| CYP2A6_exon7, 8 | exon7, 8F* | GCTCTGAGACCCCTAGATACC | 86 |
| | exon7, 8R* | GCCCCTGCTGGTGTGAGCC | 87 |
| CYP2A6_exon9 | exon9F* | GCAAGTGTACCTGGCAGGAAA | 88 |
| | exon9R* | TGTAAAATGGGCATGAACGCCC | 89 |

*Primers which are cited from article [Drug Metab. Pharmacokin, 17(5):SNP18 (482)-SNP23 (487) (2002)]

Positions of the primers and sizes of the PCR products are as shown in Table 13. Positions of nucleotide are written according to naming method of Cytochrome P450 (CYP) Allele Nomenclature Committee (http://www.cypalleles.ki.se/cyp2a6.htm).

TABLE 13
Positions of primers and sizes of PCR products

| | | | | Size |
|---|---|---|---|---|
| | Primer | | | of PCR |
| PCR products | name | References | Positions | products |
| CYP2A6_exon1 | exon1F | 76 | −764~744 | 1088 bp |
| | exon1.R | 77 | 306~324 | |
| CYP2A6_exon2 | exon2F | 78 | −151~132 | 884 bp |
| | exon2R | 79 | 713~732 | |
| CYP2A6_exon3~4 | exon3, 4F | 80 | 1504~1526 | 812 bp |
| | exon3, 4R | 81 | 2297~2315 | |
| CYP2A6_exon5 | exon5F | 82 | 3238~3261 | 402 bp |
| | exon5R | 83 | 3621~3639 | |
| CYP2A6_exon6 | exon6F | 84 | 4217~4239 | 364 bp |
| | exon6R | 85 | 4561~4580 | |
| CYP2A6_exon7, 8 | exon7, 8F | 86 | 4899~4919 | 947 bp |
| | exon7, 8R | 87 | 5827~5845 | |
| CYP2A6_exon9 | exon9F | 88 | 6082~6102 | 898 bp |
| | exon9R | 89 | 6958~6979 | |

The reaction conditions with respect to PCR fragments are as shown in Table 14.

TABLE 14
PCR reaction conditions

| PCR products | Reaction conditions |
|---|---|
| CYP2A6_exon1 | 94° C. 5 min, (94° C. 30 sec, 63° C. 30 sec, 72° C. |
| | 65 sec) 35 cycles, 72° C. 5 min |
| CYP2A6_exon2 | 94° C. 4 min, (94° C. 30 sec, 63° C. 30 sec, 72° C. |
| | 50 sec) 35 cycles, 72° C. 5 min |
| CYP2A6_exon3, 4 | 94° C.4 min, (94° C. 30 sec, 60° C. 30 sec, 72° C. |
| | 40 sec) 35 cycles, 72° C.5 min |
| CYP2A6_exon5 | 94° C.4 min, (94° C. 30 sec, 63° C. 30 sec, 72° C. |
| | 40 sec) 35 cycles, 72° C.5 min |
| CYP2A6_exon6 | 94° C. 4 min, (94° C. 30 sec, 63° C. 30 sec, 72° C. |
| | 40 sec) 35 cycles, 72° C. 5 min |
| CYP2A6_exon7, 8 | 94° C. 5 min, (94° C. 30 sec, 66° C. 30 sec, 72° C. |
| | 60 sec) 35 cycles, 72° C.5 min |
| CYP2A6_exon9 | 94° C. 4 min, (94° C. 30 sec, 66° C. 30 sec, 72° C. |
| | 60 sec) 35 cycles, 72° C. 5 min |

<5-2> Sequencing PCR Products

The PCR products which are obtained according to the exemplary embodiment <5-1> were sequenced with an automated DNA sequencer and primers having references 76 to 89.

After being compared with genetic sequences of a wild type CYP2A6 gene (reference 75), a total of 27 SNPs were found. Two of the 27 SNPs were novel. Three SNPs were discovered by structure analysis of gene deletion. A total of 30 SNPs are as shown in Table 15.

TABLE 15
variants of CYP2A6 gene found in Koreans

| | | | Amino acid | Frequency |
|---|---|---|---|---|
| SNP | Allele | Position | variants | (%) |
| −495A > G# | | Promoter | | 1.19 |
| −48T > G | | Promoter | | 28.57 |
| 13G > A | | exon 1 | G5R | 1.19 |
| 22C > T | | exon 1 | L8L | 25.00 |
| 51G > A | *1B12 | exon 1 | V17V | 14.29 |
| 144G > A | | exon 1 | Q48Q | 1.19 |
| 237G > A | | intron | | 1.19 |
| 411C > T | | intron | | 1.19 |
| 567C > T | | exon 2 | R101X | 1.19 |
| 1620T > C | | intron | | 84.52 |
| 1836G > T | | intron | | 15.48 |
| 1890G > C | | intron | | 1.19 |
| 2134A > G | | exon 4 | K194E | 2.38 |
| 3391T > C | *11 | exon 5 | S224P | 1.19 |
| 3492C > T | | exon 5 | R257R | 1.19 |
| 3570C > G | | intron | | 1.19 |
| 5336G > A | | intron | | 1.19 |
| 5628C > T | | intron | | 2.38 |
| 5636A > C | | intron | | 2.38 |
| 6218A > G | | intron | | 1.19 |
| 6282A > G | | intron | | 2.38 |
| 6293T > C | | intron | | 2.38 |
| 6354T > C | | intron | | 30.95 |
| 6458A > T# | | exon 9 | N438Y | 3.57 |
| 6558T > C | *7 | exon 9 | I471T | 20.23 |
| 6582G > T | *5 | exon 9 | G479V | 1.19 |
| 6600G > T | *8 | exon 9 | R485L | 5.95 |
| 5971G > A+ | *4 | gene deletion | | 16.00 |
| 5983T > G+ | *4 | gene deletion | | 16.00 |
| 6091C > T+ | *4 | gene deletion | | 16.00 |

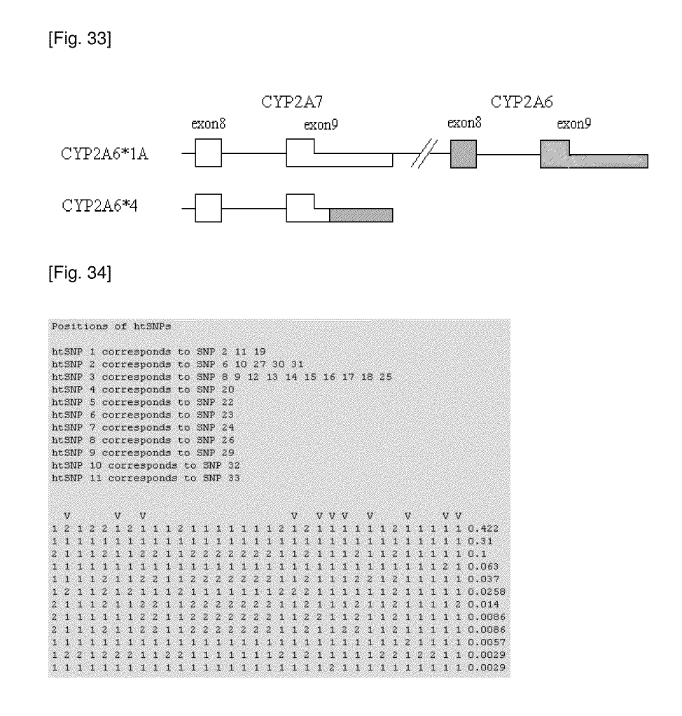

In Table 15, “+” marks variants found in a gene coupled with a part of a CYP2A6 gene and a CYP2A7 due to CYP2A6 gene deletion (refer to FIG. 33, exons 1 to 8 in the CYP2A6 gene are removed, and exon 9 of the CYP2A6 gene partly substitutes for exon 9 end of a CYP1A7 gene). The SNPs are numbered according to genetic sequences on the assumption that the CYP2A6 gene is present as a whole (since CYP2A6 gene is deleted, the SNPs are not for CYP2A6 gene).
To determine the deleted haplotypes by using the SNPs, a forward primer is designed to 5′ site within the same genetic sequences of CYP2A6 and CYP2A7 genes, and a reverse primer is designed in an exon 9 which is specific to a CYP2A6 gene and does not amplify a CYP2A7 gene. Thus, the whole CYP2A6 gene or a gene coupling with the CYP2A7 gene as the CYP2A6 gene is deleted may be amplified while the CYP2A7 is not amplified.
Bases which are specific to CYP2A6 and CYP2A7 genes are selected from the amplified PCR products. Based on translation initiation codon ATG of the CYP2A6 gene, a circumference of 6091C/T base of the CYP2A6 (reference 75) gene is similar to that of 6521T of CYP2A7 (reference 104) gene. Not only 6091, 5971G and 5983T in CYP2A6 genetic sequences are different from the CYP2A7 gene.
“*” refers to variants which are approved as allele by Internal CYP Nomenclature Committee. For example, *11 refers to a haplotype which has a variant having 224^{th} amino acid changed from serine to proline, compared with a wild type. The Internal naming method is referred to from http://www.cypalleles.ki.se/cyp2a6.htm. In the table, “#” refers to novel variants.

### Exemplary Embodiment 6

### Determining Haplotypes of Genotype of CYP2A6 Gene

The 27 CYP2A6 genetic variants and three CYP2A6 deletion tagging variants according to the exemplary embodiment 5 may possibly affect activity of CYP2A6 enzymes depending on combination thereof. Thus, the present inventors analyzed the haplotypes of the variants determined according to the exemplary embodiment 5, with SNPAlyze manufactured by DYNACOM. Typically, variants which have 5% or 10% or more frequencies are selected to predict the distribution of the haplotypes since low-frequent variants hardly secure statistical significance. However, variants which cause amino acid substitution have significant functionality even though frequencies are low. Thus, according to the present invention, six highly-frequent variants −48T>G, 22C>T, 51G>A, 1620T>C, 1836G>T, and 6354T>C, and eight variants 13G>A, 567C>T, 2134A>G, 3391T>C, 6458A>T, 6558T>C, 6582G>T and 6600G>T which cause amino acid substitution were used to determine haplotypes thereof. Variants 5971G>A, 5983T>G and 6091C>T which can label gene deletion were added to the analysis. A total of 17 variants were used to determine the haplotypes. Thus, distribution of 20 haplotypes in Koreans is as shown in FIG. 15.

### Exemplary Embodiment 7

### Selection and Verification of htSNPs

It has been reported that several haplotypes, combination of SNPs of the CYP2A6 gene, possibly affect activity of CYP2A6 enzymes. Detailed information on the produced haplotypes can be checked by a minimum marker. The minimum marker is called htSNPs which is required to mark the haplotypes accurately and includes several combinations. To select the htSNP combination, an optimal tagging set, genetic sequences of the 20 haplotypes selected according to the exemplary embodiment 6 were analyzed by SNPtagger software (http://www.well.ox.ac.uk/˜xiayi/haplotype).

As a result, the htSNP combinations were selected as shown in FIGS. 16 to 21. The selected htSNP combinations are optimal tagging sets, in which “1” refers to a wild type, “2” is a variant and “V” means selected htSNPs.

If the genotypes of the variants are determined by the htSNP analysis, the haplotypes thereof can be predicted from the analysis result. However, a combination of different haplotypes may have an identical genotype. The found htSNP combinations were analyzed by Matlab software (version 7.1, The Math Works Inc., USA) to determine diplotypes and genotypes without overlapping each other.

According to the results, the htSNPs selected according to the present embodiment may determine haplotypes without overlapping each other. That means the htSNP combinations selected according to the present invention are not identical to each other and the analysis for determining the genotypes was not incorrect at all.

### Exemplary Embodiment 8

### Rapid Search of Functional Variants in CYP2A6 Gene

Among the 27 variants in the CYP2A6 gene and three variants labeling CYP2A6 gene deletion found in Koreans and determined according to the exemplary embodiment 5, a genotype of the CYP2A6 gene which changes functionality may be used in determining gene. SNaPshot analysis, which is one of high speed genotyping technology of CYP2A6 gene, was performed to search ten functional variants at high speed. The ten functional variants include nine variants −48T>G, 13G>A, 567C>T, 2134A>G, 3391T>C, 6458A>T, 6558T>C, 6582G>T and 6600G>T which change amino acid or have proved functionality, and 6091C>T variant which labels gene deletion. The selected htSNPs include ten variants which reflect functionality, among htSNP combinations in FIG. 16. Positions of the variants are as shown in Table 17.

TABLE 17
Position of variants selected by htSNP
combinations according to the present invention

| | Variant | Position |
|---|---|---|
| htSNP 1 | SNP 1 | −48T > G |
| htSNP 2 | SNP 2 | 13G > A |
| htSNP 3 | SNP 5 | 567C > T |
| htSNP 4 | SNP 8 | 2134A > G |
| htSNP 5 | SNP 9 | 3391T > C |
| htSNP 6 | SNP 11 | 6458A > T |
| htSNP 7 | SNP 12 | 6558T > C |
| htSNP 8 | SNP 13 | 6582G > T |
| htSNP 9 | SNP 14 | 6600G > T |
| htSNP 10 | SNP 15 | 6091C > T |

More specifically, PCR was performed by using DNA of subjects as a template, and the amplified products were SNaPshot-analyzed. The primers used for PCR are as shown in Table 18.

The primers which amplify CYP2A6_long amplify full-length CYP2A6 gene. Thus, they can not apply to the CYP2A6 gene deletion. To determine 6091C>T labeling gene deletion, a pair of primers CYP2A6 delF and CYP2A6 delR should be used to amplify CYP2A6*4 products.

TABLE 18
Primer names and genetic sequences

| PCR product | Primer name | Genetic sequences(5′→3′) | reference |
|---|---|---|---|
| CYP2A6_long | CYP2A6 longF | CTCTCCCCTGGAACCCCCAG | 90 |
| | CYP2A6 longR | GCACTTATGTTTTGTGAGACATCAGAGACAA | 91 |
| CYP2A6*4 | CYP2A6 delF | AGAATCTACCCTTGAGCCAGCA | 102 |
| | CYP2A6 delR | TGTAAAATGGGCATGAACGCCC | 103 |

Reaction conditions with respect to the PCR products are as shown in Table 19.

TABLE 19
PCR reaction conditions

| PCR products | Reaction conditions |
|---|---|
| CYP2A6_long | 94° C. 1 min, (98° C. 20 sec, 62° C. 30 sec, 72° C. |
| | 7 min 30 sec) 30 cycles, 72° C. 10 min |
| CYP2A6*4 | 94° C. 5 min, (94° C. 30 sec, 58° C. 30 sec, 72° C. |
| | 1 min 20 sec) 35 cycles, 72° C. 7 min |

The remaining primers and dNTP which do not react to the amplified PCR products may affect the SNaPshot analysis. To remove the remaining primers and dNTP, 5 μl PCR product was mixed with 2 μl ExoSAP-IT (manufactured by USB) to react at 37° C. for 30 minutes, and then at 80° C. for another 15 minutes to deactivate the remaining enzymes. The enzyme-processed product was used to make multiplex SNaPshot reactant by using the primers in Table 20 to perform PCR thereto. The multiplex SNaPshot reactant and the PCR reaction conditions are shown in Tables 21 and 22.

TABLE 20
Primer names and genetic sequences thereof

| Primer name | Genetic sequences (5′→3′) | References |
|---|---|---|
| Mu_-48T > G | GGCTGGGGTGGTTTGCCTTT | 92 |
| Mu_13G > A_F | TTTTTTTTTTTTTTTTTTTTTTTTTTTTTTTTTTTTTTCTACCACC | 93 |
| | ATGCTGGCCTCA | |
| Mu_567C > T_R | TTTTTTTTTTTTTTTTTTTTTTTTTTTTTTTTTTTTTTTTTTTGAA | 94 |
| | GGTGGCTTGCTCGCCTC | |
| Mu_2134A/G_R | TTTTTTGACAGGAACTCTTTGTCCT | 95 |
| Mu_3391T/C_F | TTTTTTTTTTCCCAGCTCTATGAGATGTTC | 96 |
| Mu_6458A > T_R | TTTTTTTTTTTTTTTCAGGCCTTCTCCGAAACAGT | 97 |
| Mu_6558T/C_F | TTTTTTTTTTTTTTTTTTTTCTCCCAGTCACCTAAGGACA | 98 |
| Mu_6582G > T_F | TTTTTTTTTTTTTTTTTTTTTTTTTTTTTTTTTTCGTGTCCCCCAA | 99 |
| | ACACGTGG | |
| Mu_6600G/T_R | TTTTTTTTTTTTTTTTTTTTTTTTTGGAAGCTCATGGTGTAGTTT | 100 |
| Multi2A6/ | CAGTCATATTTGCAAGTGT | 101 |
| 7_6091C > T_F | | |

TABLE 21
Multiplex SNaPshot reactant

| | | | Volume |
|---|---|---|---|
| Composition | | | (/sample) |
| SNaPshot Multiplex Ready Reaction Mix | | | 2 |
| (ABI) | | | |
| ½ term buffer solution+ | | | 3 |
| Enzyme-processed PCR product | | | 3 |
| | Primer name | concentration | |
| Primers | Mu_−48T > G | 20 pM | 0.1 |
| | Mu_13G > A_F | 20 pM | 0.1 |
| | Mu_567C > T_R | 20 pM | 0.1 |
| | Mu_2134A/G_R | 20 pM | 0.1 |
| | Mu_3391T/C_F | 20 pM | 0.1 |
| | Mu_6458A > T_R | 20 pM | 0.1 |
| | Mu_6558T/C_F | 20 pM | 0.1 |
| | Mu_6582G > T_F | 20 pM | 0.1 |
| | Mu_6600G/T_R | 20 pM | 0.1 |

| | |
|---|---|
| Distilled water | 1.4 |
| Total | 10 |

(Composition of “+” ½ term buffer solution: 200 mM Tris-HCl, 5 mM MgCl2, pH9; Nucleic Acids Research, 30(15): 74, 2002)

TABLE 22

| PCR product | Reaction condition |
|---|---|
| SNaPshot product | (96° C. 10 sec, 50° C. 5 sec, 60° C. 30 sec) 40 cycles |

After the reaction was completed, 1 μl SAP (USB) was mixed with 10 μl SNaPshot product to react at 37° C. for 60 minutes and at 65° C. for 15 minutes, to thereby remove remaining ddNTP. Then, 0.5 μl reactant, 9.3 μl Hi-Di formamide (ABI) and 0.2 μl GeneScan-LIZ size standard material (ABI) were mixed to denature at 95° C. for five minutes. Then, the compound was analyzed by 3100 Genetic Analyzer (ABI). The analysis results are shown in FIGS. 22 to 30. The analysis of the genotypes is as shown in Table 23.

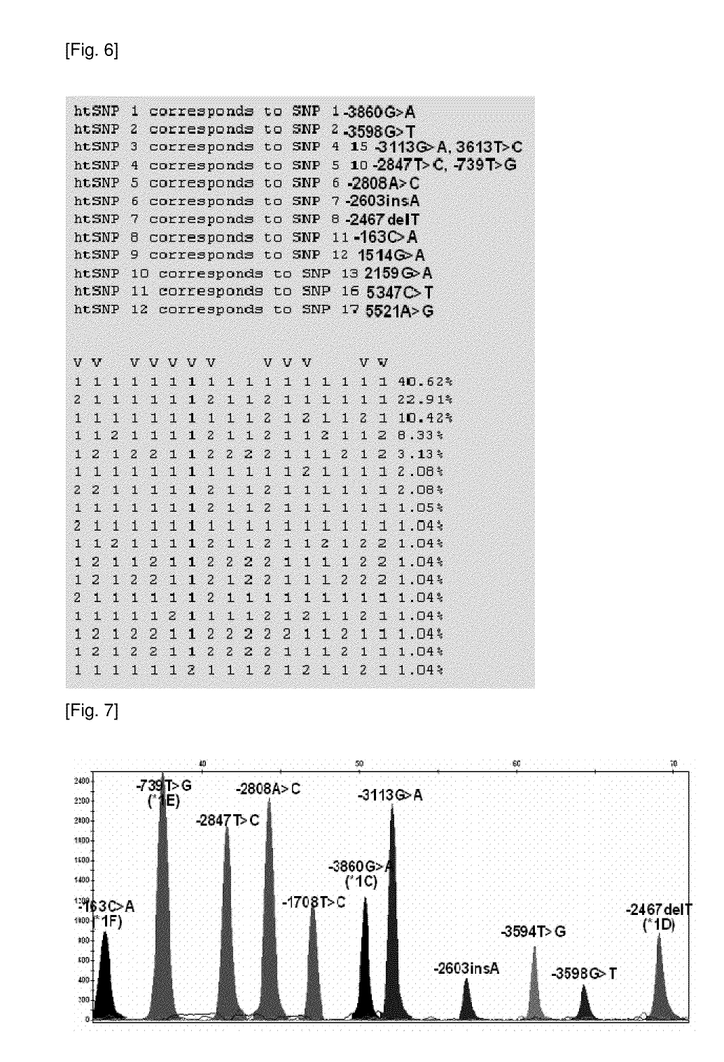

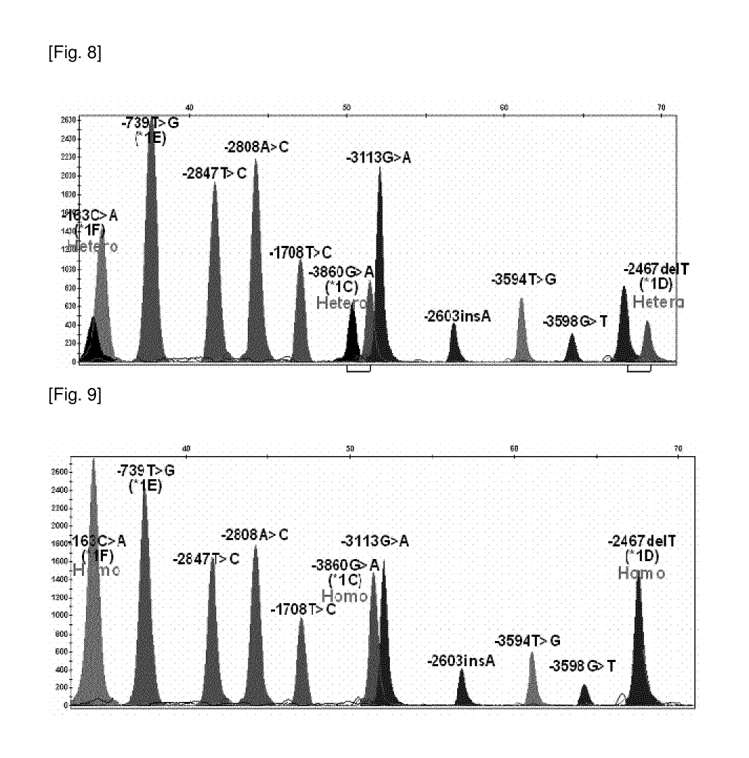

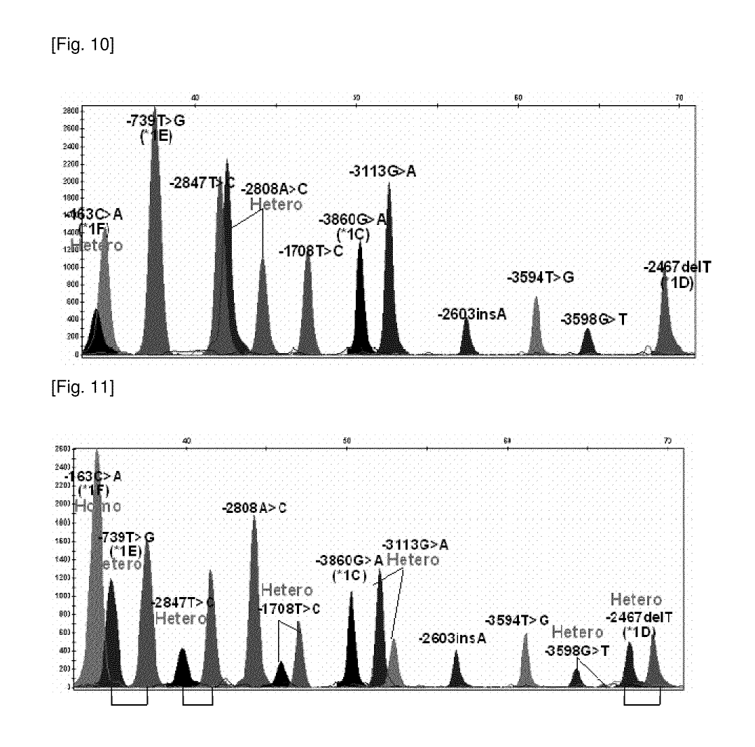

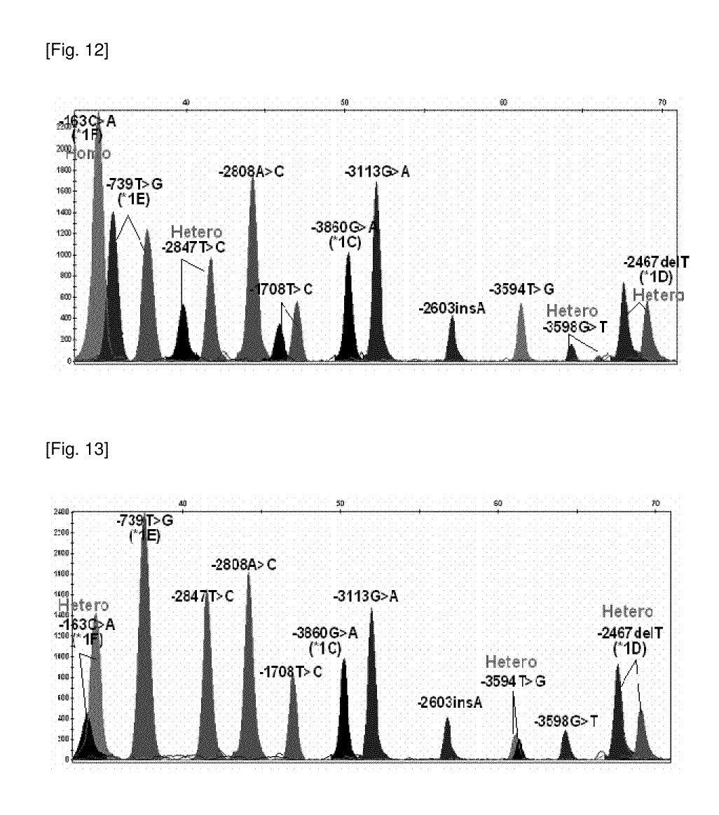

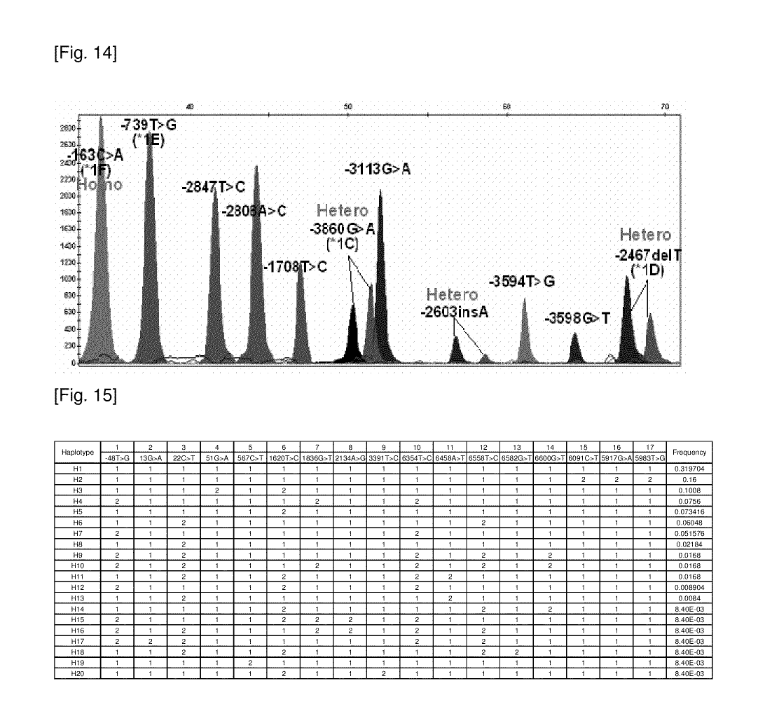

TABLE 23

| Sample # | T-48G | G13A | C567T | A2134G | T3391C | A6458T | T6558C | G6582T | G6600T | genotype |
|---|---|---|---|---|---|---|---|---|---|---|
| FIG. 8 | TG | GG | CC | AG | TT | AA | TC | GG | GG | |
| FIG. 9 | TT | GG | CT | AA | TT | AA | TT | GG | GG | |
| FIG. 10 | TT | GG | CC | AA | TT | AT | TC | GG | GG | |
| FIG. 11 | TG | GA | CC | AA | TT | AA | TC | GG | GG | |
| FIG. 12 | —T | —G | —C | —A | —C | —A | —T | —G | —G | *4/*11 |
| FIG. 13 | —G | —G | —C | —G | —T | —A | —T | —G | —G | *4/*15 |
| FIG. 14 | TG | GG | CC | AA | TT | AA | TC | GG | GT | |
| FIG. 15 | —T | —G | —C | —A | —T | —T | —T | —G | —G | *4 |
| FIG. 16 | —T | —G | —C | —A | —T | —A | —C | —T | —G | *4 |

As shown in FIGS. 22 to 30, colors and sizes of peaks are identical in each SNP. The colors and sizes of the peaks vary depending on the functional variants of the CYP2A6 gene, thereby easily identifying wild types, variants (hetero) having hetero allele and variant (homo) having homo allele. In graphs in FIGS. 22 to 30, axis X refers to moving distance of primers in an automated DNA sequencer due to length differences of primers, and axis Y refers to intensity of fluorescence emitted by a fluorescent material having specific wavelengths included in respective bases.

FIGS. 31 and 32 illustrate SNaPshot analysis which is performed together with the gene investigation in FIGS. 22 to 30, to thereby investigate CYP2A6 gene deletion other than genetic variants in FIGS. 22 to 30. FIG. 31 illustrates a CYP2A6 gene which is normally present in homologous chromosomes, and FIG. 32 illustrates a CYP2A6 which is not present in one chromosome and has only one gene.

Fifty samples were analyzed by the SNaPshot analysis developed according to the present invention and full-length sequences thereof were analyzed. According to the analysis, genotyping results were 100% identical. That means, the method according to the present invention has high reproducibility and is accurate.

Thus, the functional variants of the CYP2A6 gene may be easily determined by the analysis method according to the present invention in cost and time effective manner.

The method determines ten CYP2A6 haplotypes mainly found in Koreans and simultaneously determines the CYP2A6 genotypes by the combination at high speed. As the genetic variants found in Koreans are included, the analysis method is very accurate in determining the genotypes. Also, the method may analyze almost all of genotypes of Japanese having very similar genetic property with Koreans. Also, it is thought that the method may be used to determine CYP2A6 genotypes of Chinese within a range of 90% and more.

<CYP2D6>

### Exemplary Embodiment 9

### Determining Genotype of CYP2D6 Gene in Koreans

<9-1> Separation of Genomic DNA

Genomic DNA was separated from blood samples collected from 174 Koreans, by using a genomic DNA separation kit (Qiagen).

<9-2> Amplification of CYP2D6 Gene and Full-Length Sequencing

Fifty-one samples which were chosen randomly from the total of 174 genomic DNA samples separated according to the exemplary embodiment <9-1> were used as a template. The PCR was performed with a pair of primers to amplify nine exons and 1.8 kb promoters of a human CYP2D6 gene.

TABLE 24
Primers used for gene amplification

| Primer name | Genetic sequences (5′→3′) | References |
|---|---|---|
| CYP505 | CACTGGCTCCAAGCATGGCAG | 106 |
| 3′2D6 | ACTGAGCCCTGGGAGGTGGTA | 107 |

The PCR was performed at 94° C. for one minute, at 98° C. for ten seconds, at 64° C. for 30 seconds and at 72° C. for seven minutes for 30 cycles, and finally at 72° C. for ten minutes. As a result, PCR product which is 6,569 bp was generated (refer to Table 25).

TABLE 25
Position of primers and size of PCR product

| Primer name | Position | Size of PCR product |
|---|---|---|
| CYP505 | −1848~−1828 | 6,569 bp |
| 3′2D6 | 4732~4749 | |

The amplified PCR product was used as a template, and genetic sequences of the amplified CYP2D6 gene was analyzed by using a total of 13 primers in Table 26.

TABLE 26
Primers used for full-length sequencing

| Primer name | Genetic sequence (5′→3′) | Reference |
|---|---|---|
| CYP505 | CACTGGCTCCAAGCATGGCAG | 106 |
| 3′2D6 | ACTGAGCCCTGGGAGGTAGGTA | 107 |
| CYP507 | AACGTTCCCACCAGATTTC | 108 |
| CYP509 | GTAAGTGCCAGTGACAGATAAG | 109 |
| 2d6-11 | AGGATCCTTTGTTCAGGATATGTTGC | 110 |
| 2d6-12 | CACCAAGTACCCCACTTCCC | 111 |
| 2d6-1 | CATGTGGACTTCCAGAACACACC | 112 |
| 2d6-2 | GGTTCAAACCTTTTGCACTG | 113 |
| 2d6-3 | GTCGTGCTCAATGGGCTG | 114 |
| 2d6-4 | AAGGTGGATGCACAAAGAGT | 115 |
| 2d6-5 | GACCTAGCTCAG GAGGGACT | 116 |
| 2d6-6 | AGCTGGATGAGCTGCTAACT | 117 |
| 2d6-7 | CCTGACCTCCTCCAACATAG | 118 |
| 2d6-8 | CACCTAGTCCTCAATGCCAC | 119 |
| 2d6-9 | GAGTCTTGCAGGGGTATCAC | 120 |

<9-3> Individual Analysis According to Each Genotype

As for the remaining 123 genomic DNA samples separated according to the exemplary embodiment <9-1>, genotypes of variants *2A, *5, *2N, *10B, *14B, *18, *21B, *41A, *49, *52, and *60 which are mainly found in Asians were individually analyzed.

### a) Analysis of CYP2D6*5

To determine the CYP2D6*5 genotype, the PCR was performed by using primers in Table 27. The PCR was performed at 94° C. for one minute, at 98° C. for ten seconds, at 64° C. for 30 seconds and at 72° C. for five minutes for 30 cycles, and then at 72° C. for 10 minutes. As a result, as for a wild type, 5.1 kb PCR product including nine exons was amplified. As for the CYP2D6*5 variants, 3.5 kb PCR products were amplified.

TABLE 27
Genetic sequences and positions of primers and sizes of PCR products

| | | | | Size of PCR |
|---|---|---|---|---|
| Primer name | Genetic sequences (5′→3′) | References | Position | product |
| 5′2D6 | CCAGAAGCCTTTGCAGGCTTC | 121 | 1279~1300 | 5.1 kb |
| 3′2D6 | ACTGAGCCCTGGGAGGTGGTA | 107 | 6350~6371 | |
| 5′2D6*5 | CACCAGGCACCTGTACTCCTC | 122 | 7396~7416 | 3.5 kb |
| 3′2D6*5 | CAGGCATGAGCTAAGGCACCCAGAC | 123 | 9353~9377 | |

### b) Analysis of CYP2D6*2N

To determine the CYP2D6*2N genotype, the PCR was performed by using primers in Table 28. The PCR was performed at 94° C. for one minute, at 98° C. for ten seconds, at 64° C. for 30 seconds and at 72° C. for eight minutes for 30 cycles, and then at 72° C. for ten minutes. As a result, as for a CYP2D6*2N variant, 7.8 kb PCR product was generated.

TABLE 28
Genetic sequences and positions of primers and size of PCR product

| | | | | Size of PCR |
|---|---|---|---|---|
| Primer name | genetic Sequence (5′→3′) | reference | position | product |
| 4268Cnew | TGGGTGTTTGCTTTCCTGGTGAC | 124 | 4245~4268 | 7.8 kb |
| Primer 10B | GTGGTGGGGCATCCTCAGT | 125 | 302~321 | |

### C) Analysis of CYP2D6*2 and *41

The CYP2D6*2 genotype and CYP2D6*41 genotype include identical variants (−1235A>G; −740C>T; −678G>A; gene conversion to CYP2D7 in intron 1); 1661G>C; 2850C>T; 4180G>C) except −1584C>G variant. The genetic sequence of the variant of gene conversion to CYP2D7 in the intron 1 was analyzed with AS-PCR method (Johanson, Molecular Pharmacology, 46:452-459, 1994) by using primers in Table 29.

If the gene conversion to a CYP2D7 gene in the intron 1 occurs, the PCR is performed with a primer 9 having a reference 129 and a primer 10B having a reference 125 to generate an amplified product. If the CYP2D6*2 genotype and CYP2D6*41 genotype are normal, an amplified product is generated only when the PCR is performed with a combination of a primer 9 having a reference 129 and a primer 10 having a reference 130. Thus, the gene conversion to the CYP2D7 gene in the intron 1 may be determined by presence of the amplified product generated by the PCR with the combination of the primer 9 and the primer 10B.

The PCR was performed at 94° C. for five minutes, at 94° C. for 30 seconds, at 64° C. for 30 seconds and at 72° C. for 30 seconds for 35 cycles, and then at 72° C. for 10 minutes. A −1584C>G variant was pyrosequenced with a sequencing primer in Table 29. It was determined that −1584G is a CYP2D6*2 genotype and −1584C is a CYP2D6*41 genotype.

TABLE 29
Genetic sequence of primers

| variant | Primer name | Genetic sequence (5′→3′) | Reference |
|---|---|---|---|
| −1584C > G | F | Biotin-TCACCCCAGGAATTCAAGAC | 126 |
| | R | GGCTTCAAGCAATTCTCCTG | 127 |
| | Pyrosequencing | GTATTTTTTGTAGAGACC | 128 |
| | primer | | |

### d) Analysis of CYP2D6*10B, *14, *18 and *49 Genotypes

The CYP2D6*10B, *14, *18 and *49 genotypes were analyzed by PCR-RFLP method (Johanson, Molecular Pharmacology, 46:452-459, 1994; Wang, Drug Metabolism and Dispososition, 27:385-388, 1998; and Geadigk, Pharmacogenetics, 9:669-682, 1999). The primers used are as shown in Table 30, and experiment conditions are as shown in Table 31.

TABLE 30
Genetic sequences and positions of primers according
to each genotype

| Genotype | Primer name | Genetic sequence (5′→3′) | Reference | Position |
|---|---|---|---|---|
| *10 | Primer 9 | ACCAGCCCCCTCCACCGG | 129 | −196~197 |
| | Primer 10 | TCTGGTAGGGGAGCCTCA | 130 | 302~321 |
| | Primer 10B | GTGGTGGGGCATCCTCAGT | 125 | 302~321 |
| | Primer e | GTGGATGGTGGGGCTAATGCCTT | 131 | 1637~1659 |
| *14, *49 | Primer f | CAGAGACTCCTCGGTCTCTCGCT | 132 | 2124~2102 |
| *18 | 5′4213 | GCATCCTAGAGTCCAGTCC | 133 | 5371~5389 |
| | 3′4213 | CCTGTCTCAGCGGCCAGGCGGTGGG | 134 | 5985~6015 |

TABLE 31
restriction enzymes and RFLP phases according to each genotype

| | Size of PCR | Restriction | RFLP phase of | RFLP phase of |
|---|---|---|---|---|
| Genotype | product (bp) | enzymes | wild type (bp) | variant (bp) |
| *10B | 534 | HphI | 474 + 60 | 376 + 98 + 60 |
| *14b | 486 | MspI | 279 + 207 | 486 |
| *18 | 645 or 654 | MwoI | 348 + 258 + 39 | 272 + 258 + |
| | | | | 85 + 39 |
| *49 | 486 | Sau3A I | 347 + 142 | 286 + 142 + 61 |

### e) Analysis of CYP2D6*21, *52 and *60 Genotypes

The CYP2D6*21, *52 and *60 genotypes were analyzed by PCR-pyrosequencing. Genetic sequences of primers used for the analysis are as shown in Table 32.

TABLE 32
Genetic sequences of primers according to each genotype

| Genotype | Primer name | Genetic sequence (5′→3′) | Reference |
|---|---|---|---|
| *21B | F | biotin-TGGTGTAGGTGCTGAATGCTGT | 135 |
| | R | AGCCACTCTCACCTTCTCCATC | 136 |
| | Pyrosequencing | TCAGGTCTCGGGGGGG | 137 |
| | primer | | |
| *52 | F | Biotin-AGGCAACGACACTCATCACC | 138 |
| | R | GATACCCCTGCAAGACTCCA | 139 |
| | Pyrosequencing | GGCATCCAGGAAGTGT | 140 |
| | primer | | |
| *60 | F | biotin-ATCTCCCACCCCCAGGAC | 149 |
| | R | AGGGAGGCGATCACGTTG | 150 |
| | Pyrosequencing | GGCGATCACGTTGCT | 151 |
| | primer | | |

Data which were generated according to the exemplary embodiments <9-2> and <9-3> were analyzed based on genetic sequences of the CYP2D6 gene disclosed in GenBank Accession No. M33388. Frequencies of each allele are as shown in Table 33.

TABLE 33
Haplotypes of a CYP2D6 gene found in Koreans

| haplotype | Activity | | Frequency of |
|---|---|---|---|
| (allele) | in vivo | in vitro | allele |
| *1A | Normal | Normal | 31 |
| *2A | Normal (dx, d, s) | | 10 |
| *2XN (*2N) | Incr (d) | | 1.4 |
| *5 | None | | 6.3 |
| *10B | Decr (d) | Decr (b) | 42.2 |
| *14B | None (d) | | 0.86 |
| *18 | None (d) | Decr (d) | 0.57 |
| *21B | None | | 0.86 |
| *41A | Decr (s) | | 3.7 |
| *49 | | Decr (dx) | 2.58 |
| *52 | | Incr (dx) | 0.29 |
| *60 | | | 0.29 |

In Table 33,
“Normal” refers to a normal state,
“Incr” refers to increase,
“Decr” refers to decrease and
“None” is no activity.
Marks in the bracket are an abbreviation of labeling drugs used for the analysis:
b, bufuralol;
d, debrisoquine;
dx, dextromethorphan;
s, sparteine.

After genes were selected centering on 12 genotypes mainly found in Koreans, variants of each genotype based on Cytochrome P450 (CYP) Allele Nomenclature Committee (http://www.cypalleles.ki.se/cyp2d6.htm) are shown in Table 24. In Table 24, “1” refers to a wild type and “2” is a variant.

TABLE 34

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| SNP | −1584 | −1426 | −1245 | −1237-36 | −1235 | −1028 | −1000 | −740 | −678 | −377 | 100 | 214 | 221 | 223 | 227 | 232 |
| allele | C | C | insGA | insAA | A | T | G | C | G | A | C | G | C | C | T | G |
| *1 | 1 | 1 | 1 | 1 | 1 | 1 | 1 | 1 | 1 | 1 | 1 | 1 | 1 | 1 | 1 | 1 |
| *2A | 2 | 1 | 1 | 1 | 2 | 1 | 1 | 2 | 2 | 1 | 1 | 2 | 2 | 2 | 2 | 2 |
| *2XN | 2 | 1 | 1 | 1 | 2 | 1 | 1 | 2 | 2 | 1 | 1 | 2 | 2 | 2 | 2 | 2 |
| *5 | 1 | 1 | 1 | 1 | 1 | 1 | 1 | 1 | 1 | 1 | 1 | 1 | 1 | 1 | 1 | 1 |
| *10B | 1 | 2 | 1 | 2 | 2 | 1 | 2 | 1 | 1 | 1 | 2 | 1 | 1 | 1 | 1 | 1 |
| *14B | 2 | 1 | 1 | 1 | 1 | 1 | 1 | 2 | 2 | 1 | 1 | 2 | 2 | 2 | 2 | 2 |
| *1B | 1 | 1 | 1 | 1 | 1 | 1 | 1 | 1 | 1 | 1 | 1 | 1 | 1 | 1 | 1 | 1 |
| *21B | 2 | 1 | 1 | 1 | 2 | 1 | 1 | 2 | 2 | 1 | 1 | 2 | 2 | 2 | 2 | 2 |
| *41A | 1 | 1 | 1 | 1 | 2 | 1 | 1 | 2 | 2 | 1 | 1 | 2 | 2 | 2 | 2 | 2 |
| *49 | 1 | 2 | 1 | 1 | 2 | 1 | 2 | 1 | 1 | 1 | 2 | 1 | 1 | 1 | 1 | 1 |
| *52 | 1 | 2 | 2 | 1 | 2 | 2 | 2 | 1 | 1 | 2 | 2 | 1 | 1 | 1 | 1 | 1 |
| *60 | 1 | 1 | 1 | 1 | 1 | 1 | 1 | 1 | 1 | 1 | 1 | 1 | 1 | 1 | 1 | 1 |

| | | | | | | | | | | | | | | | | 32 | 33 |
|---|---|---|---|---|---|---|---|---|---|---|---|---|---|---|---|---|---|
| | 17 | 18 | 19 | 20 | 21 | 22 | 23 | 24 | 25 | 26 | 27 | 28 | 29 | 30 | 31 | CYP2D6 | CYP2D6 |
| SNP | 233 | 245 | 1039 | 1611 | 1661 | 1758 | 1887 | 2573 | 2850 | 2988 | 3877 | 4180 | 4125-4133 | 4388 | 4401 | deletion | duplication |
| allele | A | A | C | T | G | G | insTA | insC | C | G | G | G | ins9bp | C | C | | |
| *1 | 1 | 1 | 1 | 1 | 1 | 1 | 1 | 1 | 1 | 1 | 1 | 1 | 1 | 1 | 1 | 1 | 1 |
| *2A | 2 | 2 | 1 | 1 | 2 | 1 | 1 | 1 | 2 | 1 | 1 | 2 | 1 | 1 | 1 | 1 | 1 |
| *2XN | 2 | 2 | 1 | 1 | 2 | 1 | 1 | 1 | 2 | 1 | 1 | 2 | 1 | 1 | 1 | 1 | 2 |
| *5 | 1 | 1 | 1 | 1 | 1 | 1 | 1 | 1 | 1 | 1 | 1 | 1 | 1 | 1 | 1 | 2 | 1 |
| *10B | 1 | 1 | 2 | 1 | 2 | 1 | 1 | 1 | 1 | 1 | 1 | 2 | 1 | 1 | 1 | 1 | 1 |
| *14B | 2 | 2 | 1 | 1 | 2 | 2 | 1 | 1 | 2 | 1 | 1 | 2 | 1 | 1 | 1 | 1 | 1 |
| *1B | 1 | 1 | 1 | 1 | 1 | 1 | 1 | 1 | 1 | 1 | 1 | 1 | 2 | 1 | 1 | 1 | 1 |
| *21B | 2 | 2 | 1 | 1 | 2 | 1 | 1 | 2 | 2 | 1 | 1 | 2 | 1 | 1 | 1 | 1 | 1 |
| *41A | 2 | 2 | 1 | 1 | 2 | 1 | 1 | 1 | 2 | 2 | 1 | 2 | 1 | 1 | 1 | 1 | 1 |
| *49 | 1 | 1 | 2 | 2 | 2 | 1 | 1 | 1 | 1 | 1 | 1 | 2 | 1 | 1 | 1 | 1 | 1 |
| *52 | 1 | 1 | 2 | 1 | 2 | 1 | 1 | 1 | 1 | 1 | 2 | 2 | 1 | 2 | 2 | 1 | 1 |
| *60 | 1 | 1 | 1 | 1 | 1 | 1 | 2 | 1 | 1 | 1 | 1 | 1 | 1 | 1 | 1 | 1 | 7 |

### Exemplary Embodiment 10

### Selection and Verification of htSNPs

To determine 12 CYP2D6 genotypes that are found in Koreans according to the exemplary embodiment 9, analysis of all 33 variants in Table 34 is not cost and time effective. Thus, htSNPs, tagging genetic variants, are selected with detailed information on the haplotypes to determine the genotypes efficiently. The htSNPs are required to mark each haplotype accurately, and include various combinations. The htSNP combinations which are an optimal tagging set were selected with SNPtagger software (http://www.well.ox.ac.uk/˜xiayi/haplotype). Examples of the selected htSNP combinations are shown in FIGS. 34 to 39. The selected htSNP combinations are an optimal tagging set, in which “1” refers to a wild type and “2” is a variant, and “V” marks the selected htSNPs.

The selected combinations were analyzed with Matlab software (version 7.1, The Math Works Inc., USA) whether to determine diplotype genotypes without overlapping each other. Then, the htSNP combinations were determined.

According to the verification results, the diplotype genotypes can be determined without overlapping each other. That is, the htSNP combinations selected according to the present invention are not identical to each other, and the analysis for determining the genotype was not incorrect at all.

### Exemplary Embodiment 11

### SNaPshot Analysis

The SNaPshot analysis, one of high-speed genotyping technologies of a CYP2D6 gene, was performed by using the htSNP combinations selected according to the exemplary embodiment 10. The htSNP combinations in FIG. 34 were selected for the analysis. Positions of variants included in htSNPs are as shown in Table 35. In the case of htSNP1 to htSNP3, the genotype can be determined if one SNP of various variants is analyzed. As for htSNP9, nine bases (GTGCCCACT) are inserted and repeated. Thus, the genotype can be determined as one of 4125th base to 4133th base is analyzed and compared with a genetic sequence of a wild type gene.

TABLE 35
Position of variants selected by htSNP
combinations according to the present invention

| htSNP | Variant | Position |
|---|---|---|
| htSNP 1 | SNP 2, 7, 19 | −1426 C > T |
| htSNP 2 | SNP 3, 6, 10, 27, 30, 31 | 3877 G > A |
| htSNP 3 | SNP 8, 9, 12, 13, 14, 15, 16, 17, | 2850 C > T |
| | 18, 25 | |
| htSNP 4 | SNP 20 | 1611 T > A |
| htSNP 5 | SNP 22 | 1758 G > A |
| htSNP 6 | SNP 23 | 1887insTA |
| htSNP 7 | SNP 24 | 2573insC |
| htSNP 8 | SNP 26 | 2988 G > A |
| htSNP 9 | SNP 29 | 4125-4133 ins9 bp |
| htSNP 10 | SNP 32 | Deletion |
| htSNP 11 | SNP 33 | Duplication |

The CYP2D6 gene was amplified with the same method as in the exemplary embodiment <9-2> to generate approximately 6.7 kb product. To determine CYP2D6*5, CYP-REP-Del was amplified by using primer CYP2D6_{—}3 (5′ACCTCTCTGGGCCCTCAGGGA-3′) having a reference 154 and a primer 3′2D6*5 having a reference 123. The PCR was performed at 94° C. for one minute, at 98° C. for ten seconds, at 64° C. for 30 seconds, and at 72° C. for three minutes for 30 cycles, and finally at 72° C. for ten minutes. As a result, 6,569 bp PCR product was generated.

The remaining primers and dNTP which do not react to the amplified PCR product may affect the SNaPshot analysis. To remove the remaining primers and dNTP, 5 μl PCR product was mixed with 2 μl ExoSAP-IT (manufactured by USB) to react at 37° C. for 30 minutes, and then at 80° C. for another 15 minutes to thereby deactivate the remaining enzymes. The enzyme-processed product was mixed with a 3 μl template (mixture of 2 μl CYP2D6 gene having 6.7 kb and 1 μl CYP-REP-DEL having 3.6 kb), 1 μl SNaPshot Multiplex Reach Reaction Mix (ABI), 4 μl ½ term buffer solution (200 mM Tris HCI, 5 mM MgCl2, pH 9) and Pooled SnaPshot primer to make the overall reactant of 10 μl. Then, the PCR was performed to the reactant at 96° C. for ten seconds, at 50° C. for five seconds, at 60° C. for 30 seconds for 40 cycles. The processing concentration of the used Pooled SNaPshot primer is shown in Table 36.

TABLE 36
Genetic sequence and processing
concentration of Pooled SNaPshot primer

| | | concentration | |
|---|---|---|---|
| Primer name | Genetic sequences (5′→3′) | (M) | reference |
| 2D6 − 1426R | GCCACCACGTCTAGCTTTTT | 0.05 | 141 |
| 2D6 + 1611R (P30) | TTTTTTTTTTGGGCCCATAGCGCGCCA | 0.3 | 142 |
| | GGA | | |
| 2D6 + 1758 | CGCCTTCGCCAACCACTCC | 0.2 | 143 |
| 2D6 + 2573 (P38) | TTTTTTTTTTTTTTTTTGGGACCCAGC | 0.02 | 144 |
| | CCAGCCCCCCC | | |
| 2D6 + 2850R (P55) | TTTTTTTTTTTTTTTTTTTTTTTTTTT | 0.06 | 145 |
| | TTTTTTTTCAGGTCAGCCACCACTATG | | |
| | C | | |
| 2D6 + 2988 (P39) | TTTTTTTTTTTTTTTTTTTTAGTGCAG | 0.3 | 146 |
| | GGGCCGAGGGAG | | |
| 2D6 + 3877 (P45) | TTTTTTTTTTTTTTTTTTTTTTTTTCT | 0.3 | 147 |
| | GGGCATCCAGGAAGTGTT | | |
| 2D6 + 4125 (P50) | TTTTTTTTTTTTTTTTTTTTTTTTTTT | 0.04 | 148 |
| | TTTCAGCTTCTCGGTGCCCACTG | | |
| 2D6 + 1887R (P60) | TTTTTTTTTTTTTTTTTTTTTTTTTTT | 0.2 | 152 |
| | TTTTTTTTTTTTTAGGGAGGCGATCAC | | |
| | GTTGCT | | |
| 2D6 − 5R (P65) | TTTTTTTTTTTTTTTTTTTTTTTTTTT | 0.05 | 153 |
| | TTTTTTTTTTTTTTTTTTCTCGTCACT | | |
| | GGTCAGGGGTC | | |

After the reaction was completed, 10 μl reactant was mixed with 1 μl SAP (USB Corporation) to react at 37° C. for one hour and at 65° C. for 15 minutes. Then, 0.5 μl reactant, 0.2 μl LIZ120 (ABI) and 9.3 μl Hi-DI formamide (ABI) were mixed to be placed on a 96 well plate. After reacting at 95° C. for two minutes, the samples were analyzed by 3100 gene analyzer (ABI). The analysis result is as shown in FIG. 40.

As shown in FIG. 40, colors and sizes of peaks were identical according to each SNP. The wild type and the variant are identified clearly.

To analyze the CYP2D6 gene duplication with SNaPshot analysis, CYP-REP-Dup was amplified with the same method to generate a 3.3 kb PCR product, except usage of Dup-F_{—}2 (5′-CCTCACCACAGGACTGGCCACC-3′) having a reference 155 and Dup-R (5′-CACGTGCAGGGCACCTAGAT-3′) having a reference 156. To remove the remaining primers from the PCR product, 5 μl PCR product was mixed with 2 μl ExoSAP-IT (USB) to react at 37° C. for 30 minutes. Then, the PCR product was reacted at 80° C. for 15 minutes to deactivate the remaining ExoSAP-IT. The enzyme-processed PCR product was mixed with 3 μl template, 1 μl SNaPshot multiplex ready reaction mix, 4 μl ½ term buffer solution and SNaPshot primer having a reference 157 (CYP2D6-5R, 5′-CTCGTCACTGGTCAGGGGTC-3′) to make a 10 μl reactant to perform SNaPshot reaction with the same condition. The reactant was analyzed by 3100 gene analyzer. The analysis result is shown in FIG. 41.

As shown therein, colors and sizes of peaks are identical according to the SNPs. The wild type and the variant are identified clearly.

Fifty samples which include wild type and genetic variants were validated by sequencing. The result was 100% identical. That is, the method according to the present invention has high reproducibility and is accurate.

The method determines 12 CYP2D6 haplotypes mainly found in Koreans and at the same time determines CYP2D6 genotypes by the combinations at high speed. As the genetic variants found in Koreans are included, the method is very accurate in determining the genotypes. The method can be employed to determine genotypes of Japanese having very similar genetic property to Koreans. It is thought from the results that the method may be used to determine CYP2D6 genotypes of Chinese within a range of 90% and more.

### Exemplary Embodiment 12

### Determining Genotypes with Gene Chip

<12-1> Fabrication of Zip Code Chip

Fabrication of Probe

The probe is designed to have a complementary genetic sequence with ZipCode used for ASPE PCR reaction. Ten bp nucleotide sequence (5′-CAG GCC AAGT-3′) are inserted to 3′ as a spacer to induce hybridization with targets.

Twenty-four bp Zip Code oligonucleotide is included in 5′ of the spacer. The genetic sequence of the probe (cZip Code) is as shown in Table 37. Here, the bold letters in Table 37 are ten spacer sequences (FIG. 43).

TABLE 37

| Probe | | |
|---|---|---|
| name | Genetic sequence (5′→3′) | reference |
| cZip2 | CAGGCCAAGTATCTTGCGCGGCAGCTCG | 158 |
| | TCGACCG | |
| cZip7 | CAGGCCAAGTGTGGTCCATCACAAACAG | 159 |
| | GGAGTCG | |
| cZip8 | CAGGCCAAGTCTTGAGCGATGACGGACG | 160 |
| | GGAAAAG | |
| cZip9 | CAGGCCAAGTAAGTTGGGGATCTGTAGA | 161 |
| | CCCAGCC | |
| cZip14 | CAGGCCAAGTGGATTGCACCGTCAGCAC | 162 |
| | CACCGAG | |
| cZip15 | CAGGCCAAGTTCCCAGGACGGCGCTGGC | 163 |
| | ACGTTGA | |
| cZip16 | CAGGCCAAGTCGGCGTCCACGTCGAGTT | 164 |
| | CCTTCGC | |
| cZip19 | CAGGCCAAGTTTCGGGGAAACTCCGCAC | 165 |
| | CGCCACG | |
| cZip20 | CAGGCCAAGTTAGGTTTGCCAGTGCGTT | 166 |
| | GGATCG | |
| cZip21 | CAGGCCAAGTTCGACAACCCGGTTGGAG | 167 |
| | GATTCAG | |
| cZip22 | CAGGCCAAGTCCAAAAGCTTTACGCCAG | 168 |
| | CGCCGAA | |
| cZip24 | CAGGCCAAGTAGATCGGTGAGCAGTTCA | 169 |
| | AAGCCGG | |
| cZip27 | CAGGCCAAGTGGGTATCCGTTCGGTGTT | 170 |
| | GCGTAGT | |
| cZip31 | CAGGCCAAGTTGGTGCTGGCGCAGACCT | 171 |
| | TTGTCTC | |
| cZip32 | CAGGCCAAGTACCGCGCAAATGGACAGT | 172 |
| | GTGGCCA | |
| cZip33 | CAGGCCAAGTGACCCCAACTTGACACGT | 173 |
| | CGCAAGG | |
| cZip40 | CAGGCCAAGTCGTAAGCCTCGTCAGCTA | 174 |
| | TCCGGGG | |
| cZip41 | CAGGCCAAGTCCAAACGCACCCCAACCT | 175 |
| | GTCCGGA | |
| cZip44 | CAGGCCAAGTCGGCGGTGGCATTGTCAC | 176 |
| | TGCTGCT | |
| cZip50 | CAGGCCAAGTGCAGTTCGTGGCCATGGT | 177 |
| | GACCGCT | |
| cZip56 | CAGGCCAAGTCGTTGTGGTAGCGGCACT | 178 |
| | GGTGGTG | |
| cZip61 | CAGGCCAAGTCTGGGTGTGGGTGCTCGT | 179 |
| | ACGCCGA | |
| cZip101 | CAGGCCAAGTCGGCACATAGGACGGGGT | 180 |
| | TCAGATA | |
| cZip102 | CAGGCCAAGTGAACAAGATTGGTCCTGG | 181 |
| | AGGTGCG | |
| cZip104 | CAGGCCAAGTTCGGATGGCGTTCAGTAG | 182 |
| | GAGAAGG | |
| cZip106 | CAGGCCAAGTACACTCTCCATGCGGTAG | 183 |
| | ACCTGAC | |
| cZip109 | CAGGCCAAGTGAACCTAATGAAGACGGG | 184 |
| | GGGTGCT | |

### 2) Spotting and Fixing Probe

GAPSII glass slide which is manufactured by Corning and coated with amine was used to manufacture a chip. The glass slide was spotted with OmniGrid100 spotter by using a SMP4XP pin. The spotting condition is 22° C. and 54% humidity. Twenty-seven probes were double-spotted, respectively. After the spotting process, UV of 7,500 μJ/cm² was emitted to the glass slide to fix the probes.

### 3) Gene Chip for Zip Code Test

Eleven genotypes CYP2D6 *1, *2, *5, *10B, *14A, *14B, *18, *21, *41, *49 and *2N were verified by using nine genotype tags (SNP, marked in bold letters in Table 37) of a CYP2D6 gene.

TABLE 38

| Allele of CYP2D6 | Base change |
|---|---|
| *1 | N/A (wt) |
| *2 | 1584C > G; 1235A > G; 2850C > T; 4180G > C |
| *5 | CYP2D6 deleted |
| *10B | 100C > T; 4180G > C |
| *14A | 100C > T; 1758G > A; 2850C > T; 4180G > C |
| *14B | 1758G > A; 2850C > T; 4180G > C |
| *18 | 4125-4133insGTGCCCACT |
| *21 | 1584C > G; 1235A > G; 2573insC; 2850C > T |
| *41 | 1584C; 1235A > G; 2850C > T; 4180G > C |
| *49 | 1235A > G; 100C > T; 1611T > A; 4180G > C |
| *2N | CYP2D6 duplicated |

<12-2> Fabrication of Targets

1) Long PCR

Two micro liter CYP2D6 genomic DNA sample, 1×LA buffer solution, 2.5 mM MgCl2, 0.4 mM dNTP, 0.2 pmol/μl primers in Table 16, LA tag DNA polymerase (TAKARA: cat. No. RR002A) of 2.5 units and deionized water were mixed to make 50 μl. The mixture was then denatured once at 94° C. for one minute, and at 98° C. for ten seconds, at 64° C. for 30 seconds, at 72° C. for six minute for 30 cycles and then for another one minute to be amplified (FIG. 44). (Three types of first PCR products were generated from a CYP2D6 gene for *5, *2N allele and other allele. The condition is the same as above.)

TABLE 39
Genetic sequence of long PCR primers

| | | Genetic sequence | |
|---|---|---|---|
| Gene | Primer | (5′→3′) | Reference |
| CYP2D6 | cyp505 | CACTGGCTCCAAGCATGGCAG | 106 |
| | 3 D6 | ACTGAGCCCTGGGAGGTAGGTA | 107 |
| CYP2D6 | CYP2D6_3 | ACCTCTCTGGGCCCTCAGGGA | 154 |
| *5 | 3′2D6 *5 | CAGGCATGAGCTAAGGCACCCA | 123 |
| | | GAC | |
| CYP2D6 | Dup-F_2 | CCTCACCACAGGACTGGCCACC | 155 |
| *2N | Dup-R | CACGTGCAGGGCACCTAGAT | 156 |

2) Multiplex PCR

The generated long PCR product of 0.5 μl, 1× amplitaq buffer solution, 0.2 mM dNTP, respective primers of 0.5 pmol/μl and Ampli taq gold (Applied Biosystems: cat. No. N8080242) of 0.5 unit and deionized water were mixed to make 10 μl. The mixture was denatured once at 94° C. for five minutes, reacted at 94° C. for 45 seconds, at 57° C. for 45 seconds, at 72° C. for one minute for 30 cycles and at 72° C. for another one minute to be amplified. The second PCR includes multiplex PCR. The PCR product was amplified into four sets as shown in Table 40. The genetic sequences of the primers are as shown in Table 41.

TABLE 40
Multiplex PCR set

| Set | Template | Position | PCR product |
|---|---|---|---|
| 1 | 1st PCR product of CYP2D6 | −1584C > G | 502 bp |
| | | 100C > T | 460 bp |
| | | 1611T > A | 347 bp |
| | | 2850C > T | 477 bp |
| 2 | 1st PCR product of CYP2D6 | 1758G > A | 468 bp |
| | | 2573insC | 495 bp |
| | | 4125-4133ins9 | 484 bp |
| 3 | 1st PCR product of CYP2D6 *5 | *5 allele | 222 bp |
| 4 | 1st PCR product of CYP2D6 | *2N allele | 222 bp |
| | *2N | | |

TABLE 41
Genetic sequence of multiplex PCR primers
(genomic PCR primers)

| | primer | Genetic sequence | |
|---|---|---|---|
| Position | name | (5′→3′) | reference |
| −1584C > G | −1584 F1 | GCTGCCATACAATCCACCTG | 185 |
| | −1584 R1 | GCTCACTACAACCTTCACCTC | 186 |
| 100C > T | 100 F2 | GTCCTGCCTGGTCCTCTG | 187 |
| | 100 R2 | CTTGCCCTACTCTTCCTTGG | 188 |
| 1611T > A | 5′1611 | GTGGGCAGAGACGAGGTG | 189 |
| | 3′1611 | CGGAGTGGTTGGCGAAGG | 190 |
| 2850C > T | 1758 F1 | CTTCTCCGTGTCCACCTTG | 191 |
| | 1758 R1 | TGTCCTTTCCCAAACCCATC | 192 |
| 1758G > A | 5′ 2573 | GTCCAGGTGAACGCAGAG | 193 |
| | 3′ 2573 | CGGCAGAGAACAGGTCAG | 194 |
| 2573insC | 5′ 2850 | CAGAGATGGAGAAGGTGAGAG | 195 |
| | 3′ 2850 | TGGAGGAGGTCAGGCTTAC | 196 |
| 4125- | 4125 F2 | ACTCATCACCAACCTGTCATC | 197 |
| 4133ins9 | 4125 R2 | GGAACTACCACATTGCTTTAT | 198 |
| | | TG | |
| *5 allele | D&D-F1 | ACCTCTCTGGGCCCTCA | 199 |
| | D&D-R1 | ATGCCACCTCCTCCTTCTC | 200 |
| *2N allele | D&D-F1 | ACCTCTCTGGGCCCTCA | 199 |
| | D&D-R1 | ATGCCACCTCCTCCTTCTC | 200 |

3) ASPE (Allele Specific Primer Extension) Reaction

The generated multiplex PCR product of 6 μl, 1× amplitaq buffer solution, Cy5 dUTP (GeneChem) of 10 μM, respective ASPE primers of 125 nM, AmpliTaq gold (Applied Biosystems: cat. No. N8080242) of 1 unit, 1× Band doctor (Solgent) and deionized water were mixed to make 20 μl. The mixture was denatured once at 94° C. for five minutes, at 94° C. for 30 seconds, at 60° C. for one minute, at 72° C. for one minute for 30 cycles to be amplified (refer to FIG. 45). The ASPE reaction sets and genetic sequences of ASPE primers are as shown in Tables 42 and 43.

TABLE 42
ASPE reaction sets

| Set | Template | Position |
|---|---|---|
| 1 | 2nd PCR product of CYP2D6 1 set | −1584C > G |
| | | 100C > T |
| | | 1611T > A |
| | | 2850C > T |
| | | Positive control group |
| 2 | 2nd PCR product of CYP2D6 2 set | 1758G > A |
| | | 2573insC |
| | | 4125-4133ins9 |
| 3 | 2nd PCR product of CYP2D6 3 set | *5 allele |
| 4 | 2nd PCR product of CYP2D6 4 set | *2N allele |

TABLE 43
Genetic sequence of ASPE primers

| Position | Primer name | Genetic sequence (5′→3′) | reference |
|---|---|---|---|
| −1584C > G | −1584(C)zip15 | TCAACGTGCCAGCGCCGTCCTGGGAGCTAATTTTG | 201 |
| | | TATTTTTTGTAGAGACCG | |
| | −1584(G)zip16 | GCGAAGGAACTCGACGTGGACGCCGGCTAATTTTG | 202 |
| | | TATTTTTTGTAGAGACCC | |
| 100C > T | 100(C)Zip27 | ACTACGCAACACCGAACGGATACCCCGCTGGGCTG | 203 |
| | | CACGCTACC | |
| | 100(T)Zip2 | CGGTCGACGAGCTGCCGCGCAAGATCGCTGGGCTG | 204 |
| | | CACGCTACT | |
| 1611T > A | 1611(T)zip40 | CCCCGGATAGCTGACGAGGCTTACGCCCATAGCGC | 205 |
| | | GCCAGGAA | |
| | 1611(A)zip44 | AGCAGCAGTGACAATGCCACCGCCGCCCATAGCGC | 206 |
| | | GCCAGGAT | |
| 1758G > A | 1758(G)Zip101 | ATCTGAACCCCGTCCTATGTGCCGCCTTCTGCCCA | 207 |
| | R | TCACCCACC | |
| | 1758(A)zip109 | AGCACCCCCCGTCTTCATTAGGTTCCCTTCTGCCC | 208 |
| | | ATCACCCACT | |
| 2573insC | 2573(G)Zip9 | GGCTGGGTCTACAGATCCCCAACTTGTCAGGTCTC | 209 |
| | | GGGGGGGC | |
| | 2573(C)Zip41 | TCCGGACAGGTTGGGGTGCGTTTGGGTCAGGTCTC | 210 |
| | | GGGGGGGG | |
| 2850C > T | 2850(C)zip61 | TCGGCGTACGAGCACCCACACCCAGGAACAGGTCA | 211 |
| | | GCCACCACTATGCG | |
| | 2850(T)Zip31 | GAGACAAAGGTCTGCGCCAGCACCAGAACAGGTCA | 212 |
| | | GCCACCACTATGCA | |
| 4125- | 4125- | CTGAATCCTCCAACCGGGTTGTCGAGCTTCTCGGT | 213 |
| 4133ins9 | 4133Zip21 | GCCCACTGGA | |
| | 4125- | TTCGGCGCTGGCGTAAAGCTTTTGGGCTTCTCGGT | 214 |
| | 4133insZip22 | GCCCACTGTG | |
| *5 | 6(A)zip106 | GTCAGGTCTACCGCATGGAGAGTGTGCCCTCAGGG | 215 |
| allele | | ATGCTGCTGTA | |
| | 7(C)zip102 | CGCACCTCCAGGACCAATCTTGTTCCCCTCAGGGA | 216 |
| | | TGCTGCTGTC | |
| *2N | 7(C)zip32 | TGGCCACACTGTCCATTTGCGCGGTCCTCAGGGAT | 217 |
| allele | | GCTGCTGTC | |
| | 6(A)zip19 | CGTGGCGGTGCGGAGTTTCCCCGAACCTCAGGGAT | 218 |
| | | GCTGCTGTA | |
| Positive | 2D6pc- | CTCGGTGGTGCTGACGGTGCAATCCCCAACATGGT | 219 |
| control | 1460(zip14) | GAAACCCTATCTCTAC | |
| group | | | |

4) PCR Purification

One to four sets of PCR products which are generated by ASPE reaction were pooled and purified by Qiagene purification kit (Qiagen: ca.no.28106) according to manuals of the manufacturer. The final elution volume is 50 μl.

The purified PCR products were dried to be one to two micro liters by using a speed vacuum concentrator (module 4080C, manufactured by BioTron).

5) Prehybridization of Chip

Prehybridization buffer solution (25% formamide, 5×SSC, 0.1% SDS and 10 mg/ml BSA) was heated at 42° C. Then, the chip was dipped into the buffer solution, and cultured at 42° C. for 30 minute or more. The chip is then cleansed three times with distilled water, put into a conical tube, and dried for five minutes at 800 rpm by a centrifugal separator.

Then, the prehybridization buffer solution (25% formamide, 5×SSC, 0.1% SDS, 0.5 mg/ml poly A, 25 μg/ml Cot-1 DNA, 10% dextran sulfate) was preheated at 42° C. The dried sample was melted in the prehybridization buffer solution. The melted sample was put in a 0.5 ml PCR tube to be heated at 95° C. for five minutes. A piece of 3M paper was put in a hybridation chamber, and 3×SSC of 20 μl was dropped thereinto. After the heated sample was loaded to the prehybridized chip, the chip was assembled into the chamber and hybridized at 42° C. overnight.

The chip was then cleansed once for ten minutes by 2×SSC 0.1% SDS solution preheated to 50° C., and cleansed four times for one minute each at room temperatures. The cleansed chip was immediately put into a conical tube and dried for five minutes at 800 rpm by a centrifugal separator.

6) Analysis

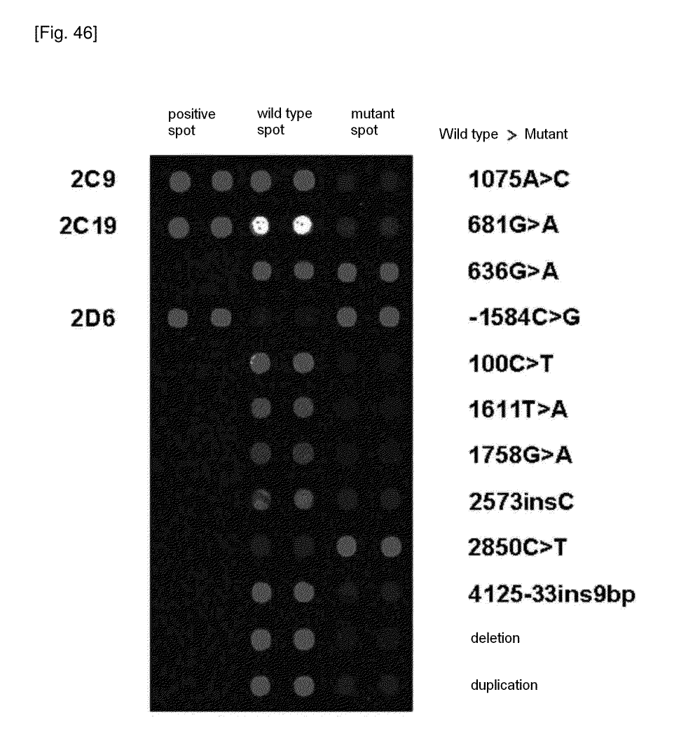

The prepared chip was scanned by GenePix 4100B scanner manufactured by Axon with output wavelength of about 650 nm. Intensity of fluorescent signals in the scanned image was analyzed by GenePix Pro 6.0 software. The analysis result is shown in FIG. 46 and Table 44.

TABLE 44

| | | | | Genetic | | Se- |
|---|---|---|---|---|---|---|
| Wild type | Mutant | In- | In- | se- | | quencing |
| spot | spot | tensity | tensity | quence | allele | result |
| −1584C | −1584G | 336 | 24343 | GG | 2D6 | *2/*2 |
| 100C | 100T | 32411 | 155 | CC | *2/*2 | |
| 1611T | 1611A | 6753 | 228 | TT | | |
| 1758G | 1758A | 3812 | 370 | WW | | |
| 2573WT | 2573insC | 5865 | 842 | WW | | |
| 2850C | 2850T | 803 | 13919 | TT | | |
| 4125WT | 4125ins9bp | 13044 | 608 | WW | | |
| del A | del C | 18326 | 381 | WW | | |
| dup CTG | dup ACA | 12457 | 553 | WW | | |

| | | |
|---|---|---|
| Positive | PC2D6 | 28948 |
| control | | |
| group | | |

As a result, the variants of the CYP2D6 gene analyzed by the gene chip were identical to those analyzed by sequencing.

<PXR>

### Exemplary Embodiment 13

### Determining Genotype of PXR Gene in Koreans

<13-1> Amplification of PXR Gene

After blood was collected from 54 healthy subjects, DNA was separated from the blood by a genomic DNA separation kit manufactured by Qiagen. The entire genetic sequences of a PXR gene were analyzed by ABI Genetic Analyzer 3130XL. As a result, six of 18 functional variants that have been reported until now were found. The PXR gene includes nine exons and is approximately 38 kb long. The PXR gene was divided into ten fragments centering on the exons having functional variants, to perform PCR thereto. The primers used for each PCR is as shown in Table 45. A, T, G and C in genetic sequences written in the present specification refer to adenine, thymine, guanine and cytosine.

TABLE 45
Primers for amplifying PXR gene and genetic sequences thereof

| PCR products | Primer name | Genetic sequence (5′→3′) | reference |
|---|---|---|---|
| PXR_5′UTR.1 | *PXR_5′UTR.1.f | CCCAGCAGTGAGCTGTGTAA | 221 |
| | *PXR_5′UTR.1.r | AGCTGAGGGCTCTTTCCTCT | 222 |
| PXR_5′UTR.2 | *PXR_5′UTR.2.f | GCACCTGCTGCTAGGGAATA | 223 |
| | *PXR_5′UTR.2.r | CTCCATTGCCCCTCCTAAGT | 224 |
| PXR_exon1 | *PXR_exon1.f | CCCCTTTTCCTGTGTTTTTG | 225 |
| | *PXR_exon1.r | CAACATTAAGTGATTGTTTTCATGC | 226 |
| PXR_exon2 | PXR_exon2F | AACAATTCCAACCCCCATTC | 227 |
| | PXR_exon2R | GGGAGCCATTTATATCCCAGA | 228 |
| PXR_exon3 | PXR_exon3F | ACTCCCACCTACACCCTTCCC | 229 |
| | PXR_exon3R | CTCTGGGAGATGGAGGGAG | 230 |
| PXR_exon4 | PXR_exon4F | AGGGGAGAATTGCTTGTCAC | 231 |
| | PXR_exon4R | AAGCTAGGCAGTTCCCCAGT | 232 |
| PXR_exon5 | PXR_exon5F | CAAGCAGGGATGTGTGTGAC | 233 |
| | PXR_exon5R | TTGGTGTCAGAAGACCCTCC | 234 |
| PXR_exon6~8 | PXR_exon6F | GGTTGTGAGGGGAGAGATGA | 235 |
| | PXR_exon8R | AAAAACACAAGCAAACAGGGGG | 236 |
| PXR_exon9 | PXR_exon9F | AAGCCTTGTCTCTTGGCTGA | 237 |
| | PXR_exon9R | TGGGCCATCTGGGGTCTATG | 238 |
| PXR_exon9.2 | *PXR_exon9.2.f | ATGTCAGAAGCTTGGCATGA | 239 |
| | *PXR_exon9.2.r | CCCACATTATTTTCCCCAGA | 240 |

*primers cited from article [Pharmacogenetics, 11:555-572 (2001)]

Positions of the primers and sizes of the PCR products are shown in Table 46. Positions of nucleotide are written according to naming method of article [HUMAN MUTATION 11:1.3 (1998)].

TABLE 46
Positions of primers and sizes of PCR products

| | | | | Size of PCR |
|---|---|---|---|---|
| PCR product | Primer name | Reference | Position | product |
| PXR_5′UTR.1 | PXR_5′UTR.1.f | 221 | −25706~25687 | 645 bp |
| | PXR_5′UTR.1.r | 222 | −25081~25062 | |
| PXR_5′UTR.2 | PXR_5′UTR.2.f | 223 | −25157~25138 | 576 bp |
| | PXR_5′UTR.2.r | 224 | −24601~24582 | |
| PXR_exon1 | PXR_exon1.f | 225 | −24601~24582 | 637 bp |
| | PXR_exon1.r | 226 | −24102~24078 | |
| PXR_exon2 | PXR_exon2F | 227 | −187~168 | 543 bp |
| | PXR_exon2R | 228 | IVS2 + 138~IVS + 158 | |
| PXR_exon3 | PXR_exon3F | 229 | IVS3 − 169~IVS − | 504 bp |
| | | | 149 | |
| | PXR_exon3R | 230 | IVS3 + 183~IVS + 201 | |
| PXR_exon4 | PXR_exon4F | 231 | IVS4 − 141~IVS4 − | 722 bp |
| | | | 121 | |
| | PXR_exon4R | 232 | IVS4 + 374~IVS4 + 393 | |
| PXR_exon5 | PXR_exon5F | 233 | IVS5 − 260~IVS5 − | 650 bp |
| | | | 241 | |
| | PXR_exon5R | 234 | IVS5 + 96~IVS5 + 115 | |
| PXR_exon6~8 | PXR_exon6F | 235 | IVS6 − 176~IVS6 − | 1214 bp |
| | | | 157 | |
| | PXR_exon8R | 236 | IVS8 + 164~IVS8 + 185 | |
| PXR_exon9 | PXR_exon9F | 237 | IVS − 123~IVS9 − | 656 bp |
| | | | 104 | |
| | PXR_exon9R | 238 | exon9 + 514~exon9 + | |
| | | | 533 | |
| PXR_exon9.2 | PXR_exon9.2.f | 239 | exon9 + 725~exon9 + | 739 bp |
| | | | 744 | |
| | PXR_exon9.2.r | 240 | IVS9 + 28~IVS9 + 46 | |

Reaction conditions with respect to each PCR fragment are as shown in Table 47.

TABLE 47
PCR reaction conditions

| PCR products | Reaction conditions |
|---|---|
| PXR_5′UTR.1 | 94° C. 4 min, (94° C. 30 sec, 55° C. 30 sec, 72° C. 35 |
| | sec) 35 cycles, 72° C. 5 min |
| PXR_5′UTR.2 | 94° C. 4 min, (94° C. 30 sec, 55° C. 30 sec, 72° C. 35 |
| | sec) 35 cycles, 72° C. 5 min |
| PXR_exon1 | 94° C. 4 min, (94° C. 30 sec, 55° C. 30 sec, 72° C. 35 |
| | sec) 35 cycles, 72° C. 5 min |
| PXR_exon2 | 94° C. 4 min, (94° C. 30 sec, 50° C. 30 sec, 72° C. 30 |
| | sec) 35 cycles, 72° C. 5 min |
| PXR_exon3 | 94° C. 4 min, (94° C. 30 sec, 54° C. 30 sec, 72° C. 30 |
| | sec) 35 cycles, 72° C. 5 min |
| PXR_exon4 | 94° C. 4 min, (94° C. 30 sec, 54° C. 30 sec, 72° C. 30 |
| | sec) 40 cycles, 72° C. 5 min |
| PXR_exon5 | 94° C.4 min, (94° C. 30 sec, 54° C. 30 sec, 72° C. 30 |
| | sec) 35 cycles, 72° C.5 min |
| PXR_exon6~8 | 94° C. 4 min, (94° C. 30 sec, 54° C. 30 sec, 72° C. |
| | 1 min 15 sec) 35 cycles, 72° C. 5 min |
| PXR_exon9 | 94° C. 4 min, (94° C. 30 sec, 55° C. 30 sec, 72° C. 35 |
| | sec) 35 cycles, 72° C. 5 min |
| PXR_exon9.2 | 94° C. 4 min, (94° C. 30 sec, 55° C. 30 sec, 72° C. 40 |
| | sec) 35 cycles, 72° C.5 min |

<13-2> PCR Product Sequencing

The genetic sequence of each PCR product generated according to the exemplary embodiment <13-1> was analyzed by an automated DNA sequencer and primers having references 131 to 150.

After being compared with genetic sequences of a wild type PXR gene (reference 130), a total of 22 SNPs were found. Among them, six SNPs are included in 18 functional variants that have been reported until now. Twenty-two SNPs are as shown in Table 48, and the reported functional variants are marked in #.

TABLE 48
Variants of PXR gene found in Koreans

| SNP | Position | rs number | Frequency (%) |
|---|---|---|---|
| −25564G > A | upstream | rs12721602 | 1.9 |
| #−25385C > T | upstream | rs3814055 | 16.7 |
| −24840A > G | upstream | | 0.9 |
| −24622A > T | upstream | | 2.8 |
| −24446C > A | upstream | rs2276705 | 2.8 |
| −24381A > C | upstream | | 21.3 |
| #−24113G > A | 5′ UTR | | 21.3 |
| 120A > G | intron 2 | | 31.5 |
| 155A > G | intron 2 | | 31.5 |
| 178A > T | intron 2 | | 0.9 |
| 2883T > G | intron 3 | rs3732356 | 3.7 |
| 4500G > A | intron 4 | | 0.9 |
| 4760G > A | intron 4 | rs3732357 | 67.6 |
| #7635A > G | intron 5 | rs6785049 | 51.9 |
| 7675C > T | intron 5 | rs6797879 | 7.4 |
| 7958C > G | intron 6 | | 0.9 |
| #8055C > T | intron 6 | rs2276707 | 37.0 |
| 8635C > A | intron 8 | | 10.2 |
| 9976G > A | 3′ UTR | rs3732358 | 0.9 |
| 10719A > G | 3′ UTR | | 5.6 |
| #11156A > C | 3′ UTR | | 48.1 |
| #11193T > C | 3′ UTR | | 48.1 |

As shown in Table 48, seven variants of the PXR gene were found in promoters, and remainders were found in 3′UTR and introns. Variants which cause amino acid substitution were not discovered.

### Exemplary Embodiment 14

### Determining Haplotypes of PXR Functional Variants

Six functional variants of the PXR gene whose functionality was investigated in the exemplary embodiment 13 may affect functionality of the PXR gene depending on combinations thereof.

Thus, the present inventors analyzed the haplotypes of variants found according to the exemplary embodiment 13 with SNPAlyze of DYNCOM. As a result, at least 14 haplotypes, which have 1% frequency and above, were confirmed as shown in Table 49.

TABLE 49

| haplotype | −25385C > T | −24113G > A | 7635A > G | 8055C > T | 11156A > C | 11193T > C | frequency |
|---|---|---|---|---|---|---|---|
| 1 | C | G | A | C | A | T | 0.3673 |
| 2 | C | G | 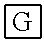 | 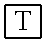 | 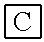 | 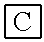 | 0.2388 |
| 3 | C | G | 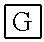 | C | A | T | 0.0694 |
| 4 | C | 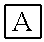 | 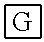 | 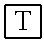 | 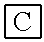 | 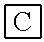 | 0.0528 |
| 5 | C | G | 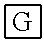 | C | 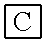 | 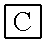 | 0.0463 |
| 6 | 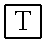 | 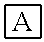 | A | C | A | T | 0.045 |
| 7 | 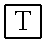 | 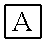 | 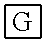 | 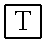 | 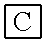 | 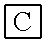 | 0.0329 |
| 8 | C | 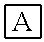 | 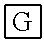 | C | 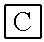 | 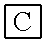 | 0.0278 |
| 9 | 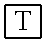 | G | 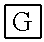 | 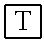 | 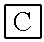 | 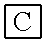 | 0.0257 |
| 10 | 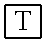 | G | A | C | A | T | 0.0203 |
| 11 | 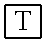 | 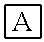 | 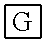 | C | 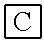 | 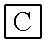 | 0.019 |
| 12 | 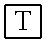 | 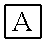 | A | C | 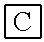 | 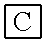 | 0.018 |
| 13 | C | G | A | 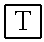 | 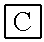 | 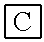 | 0.0134 |
| 14 | C | 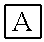 | A | C | A | T | 0.0108 |

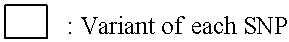

### Exemplary Embodiment 15

### Selection and Verification of htSNPs

It has been reported that several haplotypes, combination of SNPs of the PXR gene, possibly affect activity of the PXR gene. Detailed information on the produced haplotypes can be checked by a minimum marker. The minimum marker is called htSNP which is required to mark the haplotypes accurately and includes several combinations. To select the htSNP combinations, an optimal tagging set, 14 haplotypes selected in the exemplary embodiment 14 were sequenced by SNPtagger software (http://www.well.ox.ac.uk/˜xiayi/haplotype).

As a result, the htSNP combinations were selected as shown in FIG. 47. The selected htSNP combinations are one of optimal tagging sets, in which “1” refers to a wild type, “2” is a variant and “V” means selected htSNPs. The selection of htSNP combinations may vary other than the htSNP combinations in FIG. 47.

The found combinations were analyzed by Matlab software (version 7.1, The Math Works Inc., US) to determine diplotypes and genotypes without overlapping each other. The analysis results were used to determine the combinations.

According to the verification results, diplotypes and genotypes can be determined without overlapping each other. That means, the htSNP combinations selected according to the present invention are not identical to each other and the analysis for determining the genotypes was not incorrect at all.

### Exemplary Embodiment 16

### Rapid Search of Functional Variants in PXR Gene

Among the six functional variants in the PXR gene in Koreans found according to the exemplary embodiment 13, the SNaPshot analysis was performed to search functional variants affecting the PXR gene functionality at high speed. The PCR was performed by using DNA of subjects as a template, and the amplified products were SNaPshot-analyzed. The primers used for the PCR are as shown in Table 50.

TABLE 50

| PCR product | Primer name | Genetic Sequence (5′→3′) | reference |
|---|---|---|---|
| PXR_5′UTR.1 | *PXR_5′UTR.1.f | CCCAGCAGTGAGCTGTGTAA | 221 |
| | *PXR_5′UTR.1.r | AGCTGAGGGCTCTTTCCTCT | 222 |
| PXR_exon1 | *PXR_exon1.f | CCCCTTTTCCTGTGTTTTTG | 225 |
| | *PXR_exon1.r | CAACATTAAGTGATTGTTTTCATGC | 226 |
| PXR_exon6~8 | PXR_exon6F | GGTTGTGAGGGGAGAGATGA | 235 |
| | PXR_exon6R | AGCCACCTGTGGATGGTAAC | 241 |
| PXR_exon9.2 | *PXR_exon9.2.f | ATGTCAGAAGCTTGGCATGA | 239 |
| | *PXR_exon9.2.r | CCCACATTATTTTCCCCAGA | 240 |

Reaction conditions with respect to the PCR products are as shown in Table 51.

TABLE 51
PCR reaction conditions

| PCR product | Reaction conditions |
|---|---|
| PXR_5′UTR.1 | 94° C.4 min, (94° C.30 sec, 55° C.30 sec, 72° C.35 sec) |
| | 35 cycles, 72° C.5 min |
| PXR_exon1 | 94° C.4 min, (94° C.30 sec, 55° C.30 sec, 72° C.35 sec) |
| | 35 cycles, 72° C.5 min |
| PXR_exon6 | 94° C.4 min, (94° C.30 sec, 54° C.30 sec, 72° C.30 sec) |
| | 35 cycles, 72° C.5 min |
| PXR_exon9.2 | 94° C.4 min, (94° C.30 sec, 55° C.30 sec, 72° C.40 sec) |
| | 35 cycles, 72° C.5 min |

The four amplified PCR products are mixed in the same amount. The remaining primers and dNTP which do not react to the mixed PCR products may affect the SNaPshot analysis. To remove the remaining primers and dNTP, 5 μl PCR product was mixed with 2 μl ExoSAP-IT (manufactured by USB) to react at 37° C. for 30 minutes, and then at 80° C. for another 15 minutes to deactivate the remaining enzymes. The enzyme-processed product was used to make multiplex SNaPshot reactant with the primers in Table 52 to perform PCR thereto. The multiplex SNaPshot reactant and the PCR reaction conditions are shown in Tables 53 and 54.

TABLE 52
Primers and genetic sequence thereof

| Primer name | Genetic sequence (5′→3′) | Reference |
|---|---|---|
| 25385C > T_F (48) | TTTTTTTTTTTTTTTTTTTTTTTTTTTTTTTTTTGGCAATC | 242 |
| | CCAGGTT | |
| 24113G > A_F (44) | TTTTTTTTTTTTTTTTTTTTTTTTGTCTCCTCATTTCTAGG | 243 |
| | GTG | |
| 7635A > G_F (28) | TTTTTTTTCCATCCTCCCTCTTCCTCTC | 244 |
| 8055C > T_F (32) | TTTTTTTTTTTTCTGAGAAGCTGCCCCTCCAT | 245 |
| 11156A > C_F (36) | TTTTTTTTTTTTTTTTTATAAGGCATTCCACACCTA | 246 |
| 11193T > C_R (40) | TTTTTTTTTTTTTTTTTTTTATTCCTTTTGCCTTGATTTG | 157 |

TABLE 53
Multiplex SNaPshot reactant

| | | | Volume |
|---|---|---|---|
| composition | | | (/sample) |
| SNaPshot Multiplex Ready Reaction Mix (ABI) | | | 2 |
| ½ term buffer solution | | | 3 |
| Enzyme-processed PCR product | | | 3 |
| | Primer name | Concentration | |
| primers | −25385C > T_F (48) | 20 uM | 0.1 |
| | −24113G > A_F (44) | 30 uM | 0.1 |
| | 7635A > G_F (28) | 3 uM | 0.1 |
| | 8055C > T_F (32) | 7 uM | 0.1 |
| | 11156A > C_F (36) | 5 uM | 0.1 |
| | 11193T > C_R (40) | 10 uM | 0.1 |

| | |
|---|---|
| Distilled water | 1.4 |
| Total | 10 |

TABLE 54

| PCR product | Reaction conditions |
|---|---|
| SNaPshot product | (96° C. 10 sec, 50° C. 5 sec, 60° C. 30 sec) 40 cycles |

After the reaction was completed, 10 μl SNaPshot product was mixed with 1 μl SAP (USB) to react at 37° C. for one hour, and at 65° C. for 15 minutes to thereby remove [F]ddNTP. Then, 0.5 μl reactant, 9.3 μl Hi-Di formamide (ABI) and 0.2 μl GeneScan-LIZ size standard material (ABI) were mixed to be denatured at 95° C. for five minutes. The mixture was then analyzed by 3130XL Genetic Analyzer (ABI). The analysis result is shown in FIGS. 48 to 50.

As shown in FIGS. 48 to 50, colors and positions of peaks differ depending on the functional variants of the PXR gene to thereby easily identify wild types, variants (hetero) having a hetero allele and variants (homo) having homo allele. Thus, the analysis method according to the present invention may be used to analyze the functional variants in the PXR gene in a cost and time effective manner.

<UGT1A>

### Exemplary Embodiment 17

### Selection of Genetic Variants of UGT1A Genes in Koreans

Step 1) Separation of Genomic DNA

After blood was collected from 50 Koreans, genomic DNA was separated from the blood samples with a genomic DNA separation kit (Qiagen).

Step 2) Amplification of UGT1A Genes and Full-Length Sequencing

The fifty genomic DNA samples separated at step 1 were used as templates. The PCR was performed by using each of a pair of primers in Table 55 to amplify the UGT1A genes. Gene names and positions of the amplified UGT1A genes, name of used primers, genetic sequences of primers, size of primers and PCR reaction conditions are as shown in Table 55.

TABLE 55

| | | | | | Annealing |
|---|---|---|---|---|---|
| | | | Refer- | Size | temperature |
| Gene | Position | Primer name | ence | (bp) | (° C.) |
| UGT1A1 | Promoter | UGT1A1 P-For | 248 | 588 | 63 |
| | | UGT1A1 P-Rev | 249 | | |
| | exon | UGT1A1-For | 250 | 1042 | 61 |
| | | UGT1A1-Rev | 251 | | |
| UGT1A3 | Promoter | UGT1A3 P-For | 252 | 740 | 60 |
| | | UGT1A3 P-Rev | 253 | | |
| | exon | UGT1A3-For | 254 | 1203 | 61 |
| | | UGT1A3-Rev | 255 | | |
| UGT1A4 | Promoter | UGT1A4 P-For | 256 | 789 | 60 |
| | | UGT1A4 P-Rev | 257 | | |
| | exon | UGT1A4-For | 258 | 1183 | 61 |
| | | UGT1A4-Rev | 259 | | |
| UGT1A5 | Promoter | UGT1A5 P-For | 260 | 780 | 63 |
| | | UGT1A5 P-Rev | 261 | | |
| | exon | UGT1A5-For | 262 | 1171 | 61 |
| | | UGT1A5-Rev | 263 | | |
| UGT1A6 | Promoter | UGT1A6 P-For | 264 | 610 | 57 |
| | | UGT1A6 P-Rev | 265 | | |
| | exon | UGT1A6-For | 266 | 1164 | 61 |
| | | UGT1A6-Rev | 267 | | |
| UGT1A7 | Promoter | UGT1A7 P-For | 268 | 590 | 67 |
| | | UGT1A7 P-Rev | 269 | | |
| | exon | UGT1A7-For | 270 | 1278 | 61 |
| | | UGT1A7-Rev | 271 | | |
| UGT1A8 | Promoter | UGT1A8 P-For | 272 | 616 | 63 |
| | | UGT1A8 P-Rev | 273 | | |
| | exon | UGT1A8-For | 274 | 1330 | 61 |
| | | UGT1A8-Rev | 275 | | |
| UGT1A9 | Promoter | UGT1A9 P-For | 276 | 1249 | 58 |
| | | UGT1A9 P-Rev | 277 | | |
| | exon | UGT1A9-For | 278 | 1082 | 59 |
| | | UGT1A9-Rev | 279 | | |
| UGT1A10 | Promoter | UGT1A10 P-For | 280 | 620 | 65.2 |
| | | UGT1A10 P-Rev | 281 | | |
| | exon | UGT1A10-For | 282 | 1254 | 61 |
| | | UGT1A10-Rev | 283 | | |
| UGT1A | exon | Exon2-For | 284 | 329 | 58 |
| | | Exon2-Rev | 285 | | |
| UGT1A | exon | Exon3-For | 286 | 366 | 57 |
| | | Exon3-Rev | 287 | | |
| UGT1A | exon | Exon4-For | 288 | 479 | 61 |
| | | Exon4-Rev | 289 | | |
| UGT1A | exon | Exon5-For | 290 | 424 | 58 |
| | | Exon5-Rev | 291 | | |

Step 3) Analysis of Variants of UGT1A Genes

Full-length sequences of the UGT1A gene amplified at step 2 were analyzed by known 3130× Genetic Analyzer (Applied Biosystems). The analysis result was compared with genetic sequences of wild type UGT1A genes (GenBank accession No.: NT_{—}005120). The result is shown in Tables 56 and 57.

TABLE 56

| | | Nucleic | | Types | | | |
|---|---|---|---|---|---|---|---|
| | | acid | Amino acid | Wild | | | |
| Gene | position | variant | variant | type | Hetero | Homo | Frequency |
| UGT1A8 | exon 1 | A711C | T237T | 47 | 1 | 2 | 0.05 |
| | | A765G | T255T | 46 | 2 | 2 | 0.06 |
| UGT1A10 | exon 1 | C605T | T202I | 49 | 1 | 0 | 0.01 |
| | | C693T | A231A | 45 | 4 | 1 | 0.06 |
| UGT1A9 | promoter | T-440C | | 1 | 1 | 48 | 0.97 |
| | | C-331T | | 1 | 1 | 48 | 0.97 |
| | | −118insT | | 6 | 27 | 17 | 0.61 |
| | exon 1 | G588T | G196G | 97 | 3 | 0 | 0.015 |
| | | T726G | Y242X | 99 | 1 | 0 | 0.005 |
| UGT1A7 | promoter | T-382C | | 44 | 6 | 0 | 0.06 |
| | | C-341T | | 45 | 5 | 0 | 0.05 |
| | | T-57G | | 36 | 13 | 1 | 0.15 |
| | exon 1 | C33A | P11P | 33 | 14 | 3 | 0.2 |
| | | T387G | N129K | 18 | 25 | 7 | 0.39 |
| | | C391A | R131K | 18 | 25 | 7 | 0.39 |
| | | G392A | | 18 | 25 | 7 | 0.39 |
| | | T622C | W208R | 32 | 15 | 3 | 0.21 |
| | | T701C | I234T | 49 | 1 | 0 | 0.01 |
| | | G756A | L252L | 37 | 12 | 1 | 0.14 |

TABLE 57

| | | Nucleic | Amino | Types | | | fre- |
|---|---|---|---|---|---|---|---|
| | | acid | acid | Wild | | | quen- |
| Gene | position | variant | variant | type | hetero | homo | cy |
| UGT1A6 | pro- | G-427C | | 39 | 10 | 1 | 0.12 |
| | moter | | | | | | |
| | exon 1 | T19G | S7A | 34 | 15 | 1 | 0.17 |
| | | C105T | D35D | 46 | 4 | 0 | 0.04 |
| | | A315G | L105L | 43 | 7 | 0 | 0.07 |
| | | T480C | A160A | 49 | 1 | 0 | 0.01 |
| | | A541G | T181A | 36 | 14 | 0 | 0.14 |
| | | A552C | R184S | 33 | 16 | 1 | 0.18 |
| | | G627T | V209V | 46 | 4 | 0 | 0.04 |
| UGT1A5 | pro- | C-369T | | 42 | 8 | 0 | 0.08 |
| | moter | −246insC | | 42 | 8 | 0 | 0.08 |
| | exon 1 | T143C | L48S | 35 | 14 | 1 | 0.16 |
| | | C150G | D50E | 35 | 14 | 1 | 0.16 |
| | | T188C | L62P | 35 | 14 | 1 | 0.16 |
| | | C424A | H142N | 35 | 14 | 1 | 0.16 |
| | | C473G | A158G | 35 | 14 | 1 | 0.16 |
| | | C645T | L215L | 35 | 14 | 1 | 0.16 |
| | | C657T | A219A | 35 | 14 | 1 | 0.16 |
| | | C673T | H225Y | 35 | 14 | 1 | 0.16 |
| | | C742A | L248I | 35 | 14 | 1 | 0.16 |
| | | G745C | V249L | 35 | 14 | 1 | 0.16 |
| | | G775C | G259R | 35 | 14 | 1 | 0.16 |
| | | T783C | F261F | 35 | 14 | 1 | 0.16 |
| | | T792C | D264D | 25 | 19 | 6 | 0.31 |
| UGT1A4 | pro- | C-457T | | 37 | 12 | 1 | 0.14 |
| | moter | G-419A | | 37 | 12 | 1 | 0.14 |
| | | C-219T | | 37 | 12 | 1 | 0.14 |
| | | G-163A | | 37 | 12 | 1 | 0.14 |
| | exon 1 | C31T | R11W | 49 | 1 | 0 | 0.01 |
| | | T142G | L48V | 39 | 10 | 1 | 0.12 |
| | | C292T | Q98X | 49 | 1 | 0 | 0.01 |
| | | G804A | P268P | 35 | 14 | 1 | 0.16 |
| | intron 1 | C910T | | 38 | 11 | 1 | 0.13 |
| UGT1A3 | pro- | A-486G | | 48 | 2 | 0 | 0.02 |
| | moter | A-204G | | 25 | 19 | 6 | 0.31 |
| | | T-66C | | 25 | 19 | 6 | 0.31 |
| | exon 1 | T31C | W11R | 36 | 13 | 1 | 0.15 |
| | | G81A | E27E | 36 | 13 | 1 | 0.15 |
| | | C133T | R45W | 46 | 4 | 0 | 0.04 |
| | | T140C | V47A | 46 | 3 | 1 | 0.05 |
| | | A477G | A159A | 46 | 4 | 0 | 0.04 |
| | intron 1 | A918T | | 32 | 18 | 0 | 0.18 |
| UGT1A1 | pro- | C-364T | | 39 | 7 | 4 | 0.15 |
| | moter | G-64C | | 48 | 2 | 0 | 0.02 |
| | | −39insTA | | 39 | 8 | 3 | 0.14 |
| | exon 1 | G211A | G71R | 43 | 7 | 0 | 0.07 |
| | | C233T | T78M | 49 | 1 | 0 | 0.01 |
| | | C686A | P229Q | 48 | 2 | 0 | 0.02 |
| | intron 2 | T6893C | | 48 | 2 | 0 | 0.02 |
| | exon 5 | T1456G | Y486D | 49 | 1 | 0 | 0.01 |

Step 17-1) Selection of Functional Variants in UGT1A Genes

Variants which are reportedly related to increase or decrease in enzyme activity were selected based on polymorphism of the UGT1A genes found in 50 Koreans at step 3. The selected variants are shown in Table 58. Even thought it is not determined at step 3, G766A variant in a UGT1A9 gene is reportedly a functional variant in Japanese. Thus, G766A variant is included in Table 58. “Truncated protein” refers to protein whose translation is suspended due to mutants.

TABLE 58

| | | Nucleic | Activity of related | | |
|---|---|---|---|---|---|
| | | acid | enzyme reported | | |
| Gene | allele | variant | In vivo | In vitro | Frequency |
| UGT1A1 | 1A1*28 | −39insTA | Decreased | Decreased | 0.13 |
| | 1A1*6 | G211A | Decreased | Decreased | 0.06 |
| | | C233T | | Decreased | 0.01 |
| | 1A1*27 | C686A | Decreased | Decreased | 0.02 |
| UGT1A6 | 1A6*3a | T19G | | | 0.18 |
| | 1A6*5 | A541G | | | 0.14 |
| | 1A6*9 | A552C | | | 0.18 |
| UGT1A9 | 1A9*22 | −118insT | | Increased | 0.61 |
| | 1A9*4 | T726G | | truncated | 0.003 (n = 150) |
| | | | | protein | |
| | 1A9*5 | G766A | | Decreased | N.D |
| UGT1A7 | | T387G | | | 0.39 |
| | | C391A | | | 0.39 |
| | | G392A | | | 0.39 |
| | 1A7*4 | T622C | | Decreased | 0.21 |
| | | T701C | | Decreased | 0.01 |
| UGT1A4 | 1A4*4 | C31T | | | 0.01 |
| | 1A4*3b | T142G | | Decreased | 0.12 |
| | | C292T | | deleted | 0.01 |
| UGT1A3 | | A17G | | | |
| | | T31C | | | 0.15 |
| | 1A3*4 | C133T | | Decreased | 0.04 |
| | | T140C | | | 0.05 |

Step 17-2) Selection of Polymorphisms Related to Drug Sensitivity of UGT1A Gene

Polymorphisms of UGT1A1, UGT1A6 and UGT1A9 genes which are known to be involved in metabolism of irinotecan, an anti-cancer medicine for colon cancer, were selected based on polymorphism of UGT1A genes found in 50 Koreans at step 3, and are shown in Table 59. Even though a G766A variant in a UGT1A9 gene was not found at step 3, it is reportedly a functional variant in Japanese, and added to Table 59.

TABLE 59

| Gene | allele | Nucleic acid variant | Amino acid variant |
|---|---|---|---|
| UGT1A1 | 1A1*6 | G211A | G71R |
| | | C233T | T78M |
| | 1A1*27 | C686A | P229Q |
| UGT1A6 | 1A6*3a | T19G | S7A |
| | 1A6*5 | A541G | T181A |
| | 1A6*9 | A552C | R184S |
| UGT1A9 | 1A9*4 | T726G | Y242X |
| | 1A9*5 | G766A | D256N |

### Exemplary Embodiment 18

### Analysis of Functional Variants in UGT1A Genes and Polymorphisms Related to Drug Sensitivity

18-1) Analysis of Functional Variants in UGT1A Genes

The blood which was collected from subjects having wild types, variants having hetero allele and variants having homo allele of the UGT1A gene was investigated for functional variants.

Genetic sequences of UGT1A1, UGT1A3, UGT1A4, UGT1A6, UGT1A7 and UGT1A9 genes were amplified with the same methods as steps 1 and 2 according to the exemplary embodiment 17. Five micro liter PCR product of each UGT1A gene is mixed with 2 μl ExoSAP-IT (manufactured by USB) to react at 37° C. for 30 minutes to remove the remaining primers. Then, the generated reactant is reacted at 80° C. for another 15 minutes to deactivate the remaining ExoSAP-IT. The 2 μl reactant is then mixed with 1 μl SNaPshot Multiplex Ready Reaction Mix (ABI), 4 μl half term buffer solution (composition: 200 mM Tris HCl, 5 mM MgCl2, pH 9) and each SNaPshot primers in Table 60 to produce a SNaPshot reaction solution. Here, the total amount of the reactant is 10 μl.

TABLE 60

| | Nucleic | | | |
|---|---|---|---|---|
| | acid | | | Concentration |
| Gene | variant | Primer name | Reference | (pmol) |
| UGT1A1 | G211A | 1A1_G211A_F | 295 | 2 |
| | C233T | 1A1_C233T_R | 296 | 2 |
| | C686A | 1A1_C686A_R | 297 | 2 |
| UGT1A6 | T19G | 1A6_T19G_F | 298 | 2 |
| | A541G | 1A6_A541G_R | 299 | 2 |
| | A552C | 1A6_A552C_R | 300 | 2 |
| UGT1A9 | −118insT | 1A9_−118insT_R | 301 | 2 |
| | T726G | 1A9_T726G_R | 302 | 2 |
| | G766A | 1A9_G766A_F | 303 | 2 |
| UGT1A7 | T387G | 1A7_T387G_F | 304 | 2 |
| | C391A | 1A7_C391A_F | 305 | 2 |
| | G392A | 1A7_G392A_R | 306 | 2 |
| | T622C | 1A7_T622C_R | 307 | 2 |
| | T701C | 1A7_T701C_F | 308 | 2 |
| UGT1A4 | C31T | 1A4_C31T_R | 309 | 2 |
| | T142G | 1A4_T142G_R | 310 | 2 |
| | C292T | 1A4_C292T_F | 311 | 2 |
| UGT1A3 | T31C | 1A3_T31C_R | 312 | 2 |
| | C133T | 1A3_C133T_R | 313 | 2 |
| | T140C | 1A3_T140C_F | 314 | 2 |

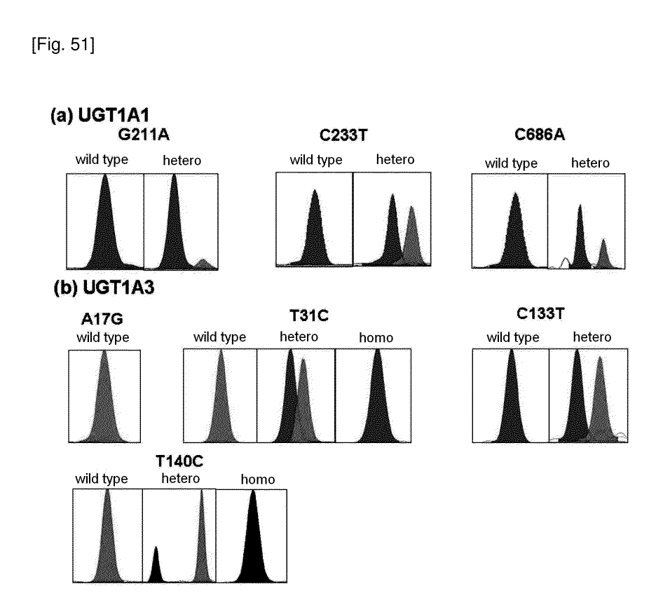

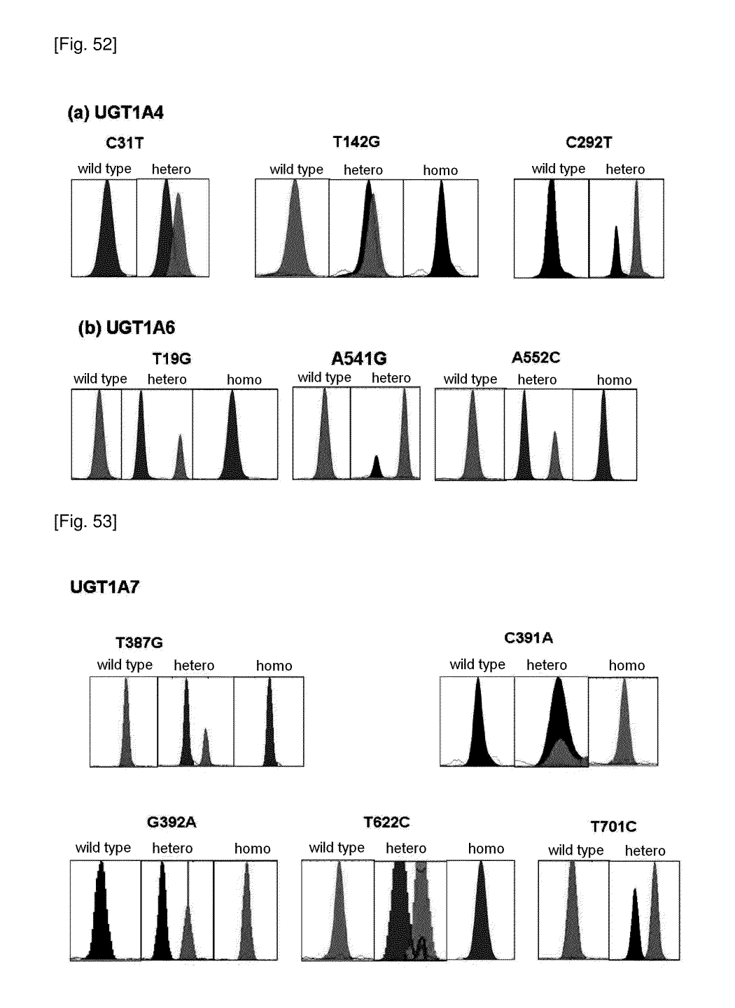

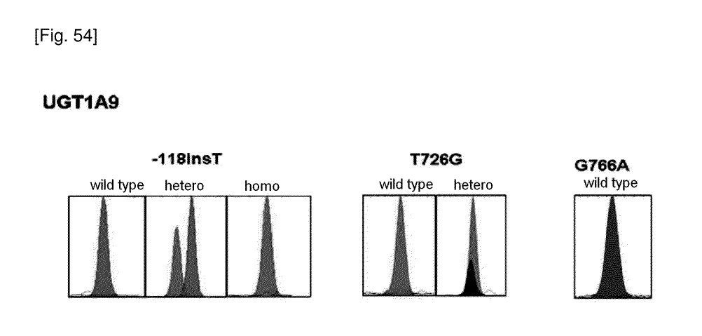

The PCR was performed to each reaction solution for 40 cycles under conditions (at 96° C. for ten seconds, at 50° C. for five seconds and at 60° C. for 30 seconds). After the reaction, 10 μl reaction solution was mixed with 1 μl SAP (shrimp alkaline phosphatase) (USB) to react at 37° C. for one hour and at 65° C. for 15 minutes. Point five micro liter reactant solution is mixed with 0.2 μl LIZ120 (ABI) and 9.3 μl Hi-Di formamide (ABI) to be placed on a 96 well plate. The reaction samples are reacted at 95° C. for two minutes, and then analyzed by 3130× Genetic Analyzer (Applied Biosystems). The analysis result is shown in FIGS. 51 to 54.

As shown in FIGS. 51 to 54, colors and positions of peaks differ depending on functional variants of each UGT1A gene to easily identify wild types, variants (hetero) having hetero allele and variants (homo) having homo allele. It was confirmed that the sizes and types of the peaks are identical 100% with the result in Table 55 obtained by sequencing according to the exemplary embodiment 17. Thus, the analysis method according to the present invention may be used to analyze the functional variants in UGT1A genes in a cost and time effective manner.

The SNaPshot analysis cannot be performed to a −39insTA genotype of a UGT1A1 gene, since it does not correspond to SNP. The variant of the −39insTA genotype was determined by PCR-pyrosequencing. Genetic sequences of primers used for the analysis are shown in Table 61. A primer UGT1A1*28 F has a biotin attached to 5′end (refer to reference 202). The primers used for pyrosequencing are referred to from article [Clin Chem., July; 49 (7):1182-5, 2003].

More specifically, PCR products which are generated by primers having references 202 and 203 were used as templates. Primers for sequencing a reference 204 are reacted to determine presence of variants with a pyrosequencer.

The generated PCR products were mixed with a 37 μl binding buffer, pH 7.6 (composition: 10 mM Tris-HCI, 2 M NaCI, 1 mM EDTA and 0.1% Tween20) and 3 μl Streptavidin Sepharose™ High performance (Amersham Bioscience). Then, the mixture was placed on a 96 well plate to react for five minutes at room temperatures at 14,000 rpm. Point three micro liter primer (100 pmol) having a reference 204 was mixed with 100 μl 1× annealing buffer, pH 7.6, (composition: 20 mM Tris acetate and 2 mM MgAc2) to be placed on a 96 well plate. The reacted sample was processed by a vacuum Prep Tool, heated at 90° C. for three minutes and cooled at room temperatures. Enzyme mixtures, substrate mixture, dATP, dCTP, dGTP and dTTP which are provided by Pyro Gold Reagent kit (Biotage) are put into the cooling plate to determine variants with a pyrosequencer.

TABLE 61

| Genotype | Primer name | Reference |
|---|---|---|
| −39insTA in | UGT1A1*28 F | 292 |
| UGT1A1 gene | UGT1A1*28 R | 293 |
| | UGT1A1*28 pyrosequencing primer | 294 |

18-2) Analysis of Functional Variants in UGT1A Genes

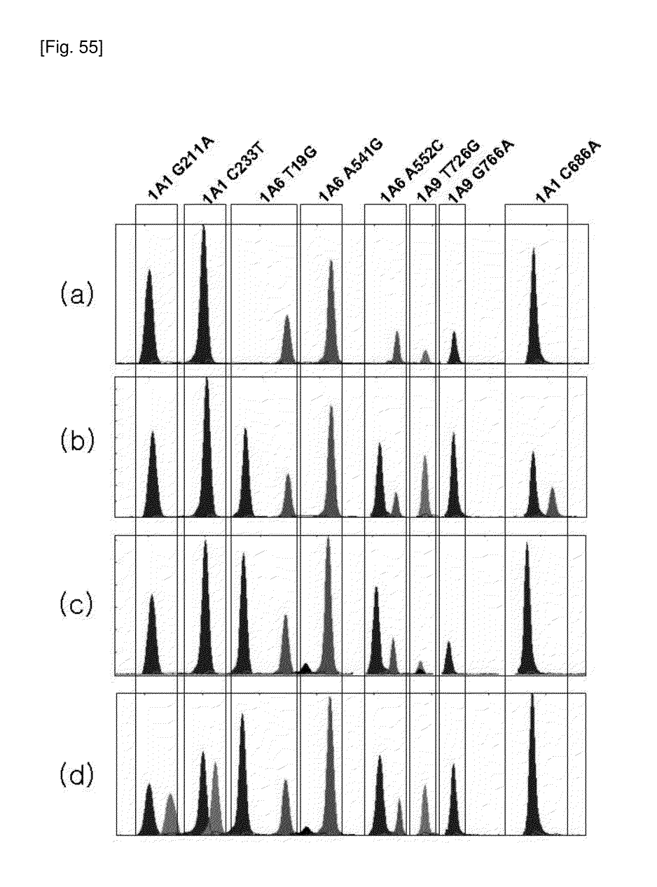

A process which is identical to the SNaPshot analysis in 18-1), except usage of primers in Table 62 instead of primers in Table 60, was performed to analyze polymorphisms of UGT1A genes related to irinotecan sensitivity. The analysis result is shown in FIG. 55. T-repeated genetic sequences in different length were attached to 5′end of primers in Table 62 to vary the length of the primers.

TABLE 62

| | | | | Concen- |
|---|---|---|---|---|
| | Nucleic acid | | | tration |
| Gene | variant | Primer name | References | (M) |
| UGT1A1 | G211A | 1A1_G211A_F | 315 | 0.1 |
| | C233T | 1A1_C233T_R(T8) | 316 | 0.1 |
| | C686A | 1A1_C686A_R(T40) | 317 | 0.1 |
| UGT1A6 | T19G | 1A6_T19G_F(T12) | 318 | 0.1 |
| | A541G | 1A6_A541G_R(T16) | 319 | 0.5 |
| | A552C | 1A6_A552C_R(T24) | 320 | 0.1 |
| UGT1A9 | T726G | 1A9_T726G_R(T28) | 321 | 0.1 |
| | G766A | 1A9_G766A_F(T32) | 322 | 0.1 |

As a result, several SNPs related to irinotecan sensitivity of UGT1A genes are easily identified at once. It was confirmed that the sizes and types of peaks are identical 100% with the result in Table 55 obtained by sequencing according to the exemplary embodiment 17.

### INDUSTRIAL APPLICABILITY

As described above, the analysis method of functional variants of CYP1A2, CYP2A6, CYP2D6, PXR and UGT1A genes or polymorphism related to drug sensitivity according to the present invention may be used to determine the functional variants of CYP1A2, CYP2A6, CYP2D6, PXR and UGT1A genes or polymorphisms related to drug sensitivity of UGT1A genes by using an optimal search set based on polymorphisms of CYP1A2, CYP2A6, CYP2D6, PXR and UGT1A genes of Koreans that have not been determined yet. The present invention may be applicable to determine genotypes of CYP1A2, CYP2A6, CYP2D6, PXR and UGT1A genes in Asians such as Japanese and Chinese having similar genetic property to Koreans, as well as Koreans.

Although a few exemplary embodiments of the present invention have been shown and described, it will be appreciated by those skilled in the art that changes may be made in these exemplary embodiments without departing from the principles and spirit of the invention, the scope of which is defined in the appended claims and their equivalents.

What is claimed is:

1. A method of selecting htSNPs of a human CYP2D6 gene, the method comprising:
(a) collecting a biological sample from humans;
(b) extracting nucleic acid from the sample collected at operation (a);
(c) performing PCR with a primer which amplifies a human CYP2D6 gene or a fragment thereof by using the nucleic acid extracted at operation (b) as a template;
(d) determining a presence of variants in genetic sequences of a PCR product obtained at operation (c);
(e) determining a haplotype from the genetic sequences of the PCR product that is determined to have the variant at operation (d); and
(f) sequencing the haplotype determined at operation (e) with SNPtagger software and selecting htSNPs.

2. The method according to claim 1, wherein the biological sample at operation (a) is selected from blood, skin cells, mucous cells and hair.

3. The method according to claim 1, wherein the primer at operation (c) comprises a genetic sequence selected from references 106, 107, 121 to 127, 129 to 136, 138, 139, 149 and 150.

4. The method according to claim 1, wherein the variant at operation (d) is selected from SNP, gene deletion and gene duplication.

5. The method according to claim 1, wherein the determining the presence of the variants at operation (d) comprises determining the presence of the variants with one of sequencing, electrophoretic analysis and RFLP analysis.

6. The method according to claim 1, further comprising repeating the operations (a) to (d).

7. A method of determining a genotype of a human CYP2D6 gene, the method comprising:
(a) collecting a biological sample from humans;
(b) extracting nucleic acid from the sample collected at operation (a);
(c) performing PCR with a primer which amplifies a human CYP2D6 gene or a fragment thereof by using the nucleic acid extracted at operation (b) as a template; and
(d) determining a presence of at least 11 variants in a CYP2A6 gene including one from −1426C>T, 100C>T and 1039C>T; one from −1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 2988G>A; 4125-4133insGTGCCCACT; 2D6 deletion; and 2D6 duplication.

8. The method according to claim 7, wherein the biological sample at operation (a) is selected from blood, skin cells, mucous cells and hair.

9. The method according to claim 7, wherein the primer at operation (c) comprises a genetic sequence selected from references 106, 107, 121 to 127, 129 to 136, 138, 139, 149 and 150.

10. The method according to claim 7, wherein the operation (d) comprises determining a presence of variants including one from −1426C>T, 100C>T and 1039C>T; one from 1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 2988G>A; 4125-4133insGTGCCCACT; 2D6 deletion; and 2D6 duplication.

11. The method according to claim 7, wherein the operation (d) comprises determining a presence of variants including one from −1584C>G; −1426C>T, 100C>T and 1039C>T; one from 1611T>A; 1758G>A; 2573insC; −740C>T, −678G>A, 214G>C, 221C>A, 223C>G, 227T>C, 232G>C, 233A>C, 245A>G and 2850C>T; one from −1245insGA, −1028T>C, −377A>C, 3877G>A, 4388C>T and 4401C>T; 4125-4133insGTGCCCACT; 2D6 deletion; and 2D6 duplication.

12. The method according to claim 7, wherein the operation (d) comprises determining a presence of variants including one from −1426C>T, 100C>T and 1039C>T; one from 1584C>G; −1028T>C, −377A>G, 3877G>A, 4388C>T and 4401C>T; one from −740C>T, −678G>A, 214G>C, 221C>A, 223C>G, 227T>C, 232G>C, 233A>C, 245A>G and 2850C>T; 1611T>A; 1758G>A; 1887insTA; 2573insC; 4125-4133insGTGCCCACT; 2D6 deletion; and 2D6 duplication.

13. The method according to claim 7, wherein the operation (d) comprises determining a presence of variants including one from −1584C>G; −1426C>T, 100C>T and 1039C>T; one from 1611T>A; 1758G>A; 2573insC; −740C>T, −678G>A, 214G>C, 221C>A, 223C>G, 227T>C, 232G>C, 233A>C, 245A>G and 2850C>T; one from −1245insGA, −1028T>C, −377A>G, 3877G>A, 4388C>T and 4401C>T; 4125-4133insGTGCCCACT; −1235A>G; 1887insTA; 2D6 deletion; and 2D6 duplication.

14. The method according to claim 7, wherein the operation (d) comprises determining a presence of variants including one from −1426C>T, 100C>T and 1039C>T; one from 1028T>C, −377A>G, 3877G>A, 4388C>T and 4401C>T; one from 1611T>A; 1661G>C and 4180G>C; 1758G>A; 1887insTA; 2573insC; 2988G>A; 4125-4133insGTGCCCACT; −1235A>G; 1887insTA; 2D6 deletion; and 2D6 duplication.

15. The method according to claim 7, wherein the operation (d) comprises determining a presence of variants including one from −1584C>G; −1426C>T, 100C>T and 1039C>T; one from 1611T>A; 1758G>A; 2573insC; −740C>T, −678G>A, 214G>C, 221C>A, 223C>G, 227T>C, 232G>C, 233A>C, 245A>G and 2850C>T; one from −1245insGA, −1028T>C, −377A>G, 3877G>A, 4388C>T and 4401C>T; 1887insTA; 2988G>A; 4125-4133insGTGCCCACT; 2D6 deletion; and 2D6 duplication.

16. The method according to claim 7, wherein the determining the presence of the variants at operation (d) comprises determining the presence of the variants with SNaPshot analysis.

17. The method according to claim 16, wherein the SNaPshot analysis is performed with a primer which has a base right next to a SNP as 3′end, has a genetic sequence annealed adjacent to the SNP site, and has a T base added to 5′end.

18. The method according to claim 17, wherein the primer comprises genetic sequences selected from references 141 to 148, 152 and 153.

19. A method of determining a human CYP2D6 gene by using a gene chip, the method comprising:
(a) extracting a gene to be investigated and performing multiplex PCR to receive a PCR product having a SNP circumference to be identified;
(b) performing ASPE reaction to an ASPE (allele specific primer extension) primer to identify a specific base of each allele;
(c) mixing the reactant to the gene chip; and
(d) analyzing the chip.

20. The method according to claim 19, wherein the gene chip comprises a probe which has a genetic sequence with references 158 to 184.


SEQUENCE LISTING (115 sequences)

SEQ ID NO: 1 - DNA, 6597 bp, Homo sapiens
cactggctccaagcatggcagctgccatacaatccacctgtagagggcccggtcctcctgtcctcagtggatgatcccgtagaagtccagagctcggcagctgccctcccacaaaagacaggattttgaaagcagcaagagagaagagacgtatcaggtagtcacagtggctcaggcctgtaatcccagcactttgggaggcccaggtgggaggatcgcttcaccccaggaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgaggctggcgcatttcccagctggaatccggtgtcgaagtggggggcggggaccgcacctgtgctgtaagctcagtgtgggtggcgcggggcccgcggggtcttccctgagtgcaaaggcggtcagggtgggcagagacgaggtggggcaaagccctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggct

SEQ ID NO: 2 - DNA, 21 bp, Artificial Sequence
  CYP505 primer
cactggctccaagcatggcag

SEQ ID NO: 3 - DNA, 21 bp, Artificial Sequence
  3′2D6 primer
actgagccctgggaggtggta

SEQ ID NO: 4 - DNA, 19 bp, Artificial Sequence
  CYP507 primer
aacgttcccaccagatttc

SEQ ID NO: 5 - DNA, 22 bp, Artificial Sequence
  CYP509 primer
gtaagtgccagtgacagataag

SEQ ID NO: 6 - DNA, 26 bp, Artificial Sequence
  2d6-11 primer
aggatcctttgttcaggatatgttgc

SEQ ID NO: 7 - DNA, 20 bp, Artificial Sequence
  2d6-12 primer
caccaagtaccccacttccc

SEQ ID NO: 8 - DNA, 23 bp, Artificial Sequence
  2d6-1 primer
catgtggacttccagaacacacc

SEQ ID NO: 9 - DNA, 20 bp, Artificial Sequence
  2d6-2 primer
ggttcaaaccttttgcactg

SEQ ID NO: 10 - DNA, 18 bp, Artificial Sequence
  2d6-3 primer
gtcgtgctcaatgggctg

SEQ ID NO: 11 - DNA, 20 bp, Artificial Sequence
  2d6-4 primer
aaggtggatgcacaaagagt

SEQ ID NO: 12 - DNA, 20 bp, Artificial Sequence
  2d6-5 primer
gacctagctcaggagggact

SEQ ID NO: 13 - DNA, 20 bp, Artificial Sequence
  2d6-6 primer
agctggatgagctgctaact

SEQ ID NO: 14 - DNA, 20 bp, Artificial Sequence
  2d6-7 primer
cctgacctcctccaacatag

SEQ ID NO: 15 - DNA, 20 bp, Artificial Sequence
  2d6-8 primer
cacctagtcctcaatgccac

SEQ ID NO: 16 - DNA, 20 bp, Artificial Sequence
  2d6-9 primer
gagtcttgcaggggtatcac

SEQ ID NO: 17 - DNA, 21 bp, Artificial Sequence
  5′2D6 primer
ccagaagcctttgcaggcttc

SEQ ID NO: 18 - DNA, 21 bp, Artificial Sequence
  5′2D6*5 primer
caccaggcacctgtactcctc

SEQ ID NO: 19 - DNA, 25 bp, Artificial Sequence
  3′2D6*5 primer
caggcatgagctaaggcacccagac

SEQ ID NO: 20 - DNA, 23 bp, Artificial Sequence
  4268Cnew primer
tgggtgtttgctttcctggtgac

SEQ ID NO: 21 - DNA, 19 bp, Artificial Sequence
  Primer 10B
gtggtggggcatcctcagt

SEQ ID NO: 22 - DNA, 20 bp, Artificial Sequence
  F primer for -1584C>G
tcaccccaggaattcaagac

SEQ ID NO: 23 - DNA, 20 bp, Artificial Sequence
  R primer for -1584C>G
ggcttcaagcaattctcctg

SEQ ID NO: 24 - DNA, 18 bp, Artificial Sequence
  pyrosequencing primer for -1584C>G
gtattttttgtagagacc

SEQ ID NO: 25 - DNA, 18 bp, Artificial Sequence
  Primer 9
accagccccctccaccgg

SEQ ID NO: 26 - DNA, 18 bp, Artificial Sequence
  Primer 10
tctggtaggggagcctca

SEQ ID NO: 27 - DNA, 23 bp, Artificial Sequence
  Primer e
gtggatggtggggctaatgcctt

SEQ ID NO: 28 - DNA, 23 bp, Artificial Sequence
  Primer f
cagagactcctcggtctctcgct

SEQ ID NO: 29 - DNA, 19 bp, Artificial Sequence
  5′4213 primer
gcatcctagagtccagtcc

SEQ ID NO: 30 - DNA, 25 bp, Artificial Sequence
  3′4213 primer
cctgtctcagcggccaggcggtggg

SEQ ID NO: 31 - DNA, 22 bp, Artificial Sequence
  F primer for *21B genotype
tggtgtaggtgctgaatgctgt

SEQ ID NO: 32 - DNA, 22 bp, Artificial Sequence
  R primer for *21B genotype
agccactctcaccttctccatc

SEQ ID NO: 33 - DNA, 16 bp, Artificial Sequence
  pyrosequencing primer for *21B genotype
tcaggtctcggggggg

SEQ ID NO: 34 - DNA, 20 bp, Artificial Sequence
  F primer for *52 genotype
aggcaacgacactcatcacc

SEQ ID NO: 35 - DNA, 20 bp, Artificial Sequence
  R primer for *52 genotype
gatacccctgcaagactcca

SEQ ID NO: 36 - DNA, 16 bp, Artificial Sequence
  pyrosequencing primer for *52 genotype
ggcatccaggaagtgt

SEQ ID NO: 37 - DNA, 20 bp, Artificial Sequence
  2D6-1426R primer
gccaccacgtctagcttttt

SEQ ID NO: 38 - DNA, 30 bp, Artificial Sequence
  2D6+1611R (P30) primer
ttttttttttgggcccatagcgcgccagga

SEQ ID NO: 39 - DNA, 19 bp, Artificial Sequence
  2D6+1758 primer
cgccttcgccaaccactcc

SEQ ID NO: 40 - DNA, 38 bp, Artificial Sequence
  2D6+2573 (P38) primer
tttttttttttttttttgggacccagcccagccccccc

SEQ ID NO: 41 - DNA, 55 bp, Artificial Sequence
  2D6+2850R (P55) primer
tttttttttttttttttttttttttttttttttttcaggtcagccaccactatgc

SEQ ID NO: 42 - DNA, 39 bp, Artificial Sequence
  2D6+2988 (P39) primer
ttttttttttttttttttttagtgcaggggccgagggag

SEQ ID NO: 43 - DNA, 45 bp, Artificial Sequence
  2D6+3877 (P45) primer
tttttttttttttttttttttttttctgggcatccaggaagtgtt

SEQ ID NO: 44 - DNA, 50 bp, Artificial Sequence
  2D6+4125 (P50) primer
ttttttttttttttttttttttttttttttcagcttctcggtgcccactg

SEQ ID NO: 45 - DNA, 18 bp, Artificial Sequence
  F primer for *60 genotype
atctcccacccccaggac

SEQ ID NO: 46 - DNA, 18 bp, Artificial Sequence
  R primer for *60 genotype
agggaggcgatcacgttg

SEQ ID NO: 47 - DNA, 15 bp, Artificial Sequence
  pyrosequencing primer for *60 genotype
ggcgatcacgttgct

SEQ ID NO: 48 - DNA, 60 bp, Artificial Sequence
  2D6+1887R (P60) primer
ttttttttttttttttttttttttttttttttttttttttagggaggcgatcacgttgct

SEQ ID NO: 49 - DNA, 65 bp, Artificial Sequence
  2D6-5R (P65) primer
tttttttttttttttttttttttttttttttttttttttttttttctcgtcactggtcaggggtc

SEQ ID NO: 50 - DNA, 21 bp, Artificial Sequence
  CYP2D6_3 primer
acctctctgggccctcaggga

SEQ ID NO: 51 - DNA, 22 bp, Artificial Sequence
  Dup-F_2 primer
cctcaccacaggactggccacc

SEQ ID NO: 52 - DNA, 20 bp, Artificial Sequence
  Dup-R primer
cacgtgcagggcacctagat

SEQ ID NO: 53 - DNA, 20 bp, Artificial Sequence
  CYP2D6-5R primer
ctcgtcactggtcaggggtc

SEQ ID NO: 54 - DNA, 35 bp, Artificial Sequence
  cZip2 probe
caggccaagtatcttgcgcggcagctcgtcgaccg

SEQ ID NO: 55 - DNA, 35 bp, Artificial Sequence
  cZip7 probe
caggccaagtgtggtccatcacaaacagggagtcg

SEQ ID NO: 56 - DNA, 35 bp, Artificial Sequence
  cZip8 probe
caggccaagtcttgagcgatgacggacgggaaaag

SEQ ID NO: 57 - DNA, 35 bp, Artificial Sequence
  cZip9 probe
caggccaagtaagttggggatctgtagacccagcc

SEQ ID NO: 58 - DNA, 35 bp, Artificial Sequence
  cZip14 probe
caggccaagtggattgcaccgtcagcaccaccgag

SEQ ID NO: 59 - DNA, 35 bp, Artificial Sequence
  cZip15 probe
caggccaagttcccaggacggcgctggcacgttga

SEQ ID NO: 60 - DNA, 35 bp, Artificial Sequence
  cZip16 probe
caggccaagtcggcgtccacgtcgagttccttcgc

SEQ ID NO: 61 - DNA, 35 bp, Artificial Sequence
  cZip19 probe
caggccaagtttcggggaaactccgcaccgccacg

SEQ ID NO: 62 - DNA, 34 bp, Artificial Sequence
  cZip20 probe
caggccaagttaggtttgccagtgcgttggatcg

SEQ ID NO: 63 - DNA, 35 bp, Artificial Sequence
  cZip21 probe
caggccaagttcgacaacccggttggaggattcag

SEQ ID NO: 64 - DNA, 35 bp, Artificial Sequence
  cZip22 probe
caggccaagtccaaaagctttacgccagcgccgaa

SEQ ID NO: 65 - DNA, 35 bp, Artificial Sequence
  cZip24 probe
caggccaagtagatcggtgagcagttcaaagccgg

SEQ ID NO: 66 - DNA, 35 bp, Artificial Sequence
  cZip27 probe
caggccaagtgggtatccgttcggtgttgcgtagt

SEQ ID NO: 67 - DNA, 35 bp, Artificial Sequence
  cZip31 probe
caggccaagttggtgctggcgcagacctttgtctc

SEQ ID NO: 68 - DNA, 35 bp, Artificial Sequence
  cZip32 probe
caggccaagtaccgcgcaaatggacagtgtggcca

SEQ ID NO: 69 - DNA, 35 bp, Artificial Sequence
  cZip33 probe
caggccaagtgaccccaacttgacacgtcgcaagg

SEQ ID NO: 70 - DNA, 35 bp, Artificial Sequence
  cZip40 probe
caggccaagtcgtaagcctcgtcagctatccgggg

SEQ ID NO: 71 - DNA, 35 bp, Artificial Sequence
  cZip41 probe
caggccaagtccaaacgcaccccaacctgtccgga

SEQ ID NO: 72 - DNA, 35 bp, Artificial Sequence
  cZip44 probe
caggccaagtcggcggtggcattgtcactgctgct

SEQ ID NO: 73 - DNA, 35 bp, Artificial Sequence
  cZip50 probe
caggccaagtgcagttcgtggccatggtgaccgct

SEQ ID NO: 74 - DNA, 35 bp, Artificial Sequence
  cZip56 probe
caggccaagtcgttgtggtagcggcactggtggtg

SEQ ID NO: 75 - DNA, 35 bp, Artificial Sequence
  cZip61 probe
caggccaagtctgggtgtgggtgctcgtacgccga

SEQ ID NO: 76 - DNA, 35 bp, Artificial Sequence
  cZip101 probe
caggccaagtcggcacataggacggggttcagata

SEQ ID NO: 77 - DNA, 35 bp, Artificial Sequence
  cZip102 probe
caggccaagtgaacaagattggtcctggaggtgcg

SEQ ID NO: 78 - DNA, 35 bp, Artificial Sequence
  cZip104 probe
caggccaagttcggatggcgttcagtaggagaagg

SEQ ID NO: 79 - DNA, 35 bp, Artificial Sequence
  cZip106
caggccaagtacactctccatgcggtagacctgac

SEQ ID NO: 80 - DNA, 35 bp, Artificial Sequence
  cZip109 probe
caggccaagtgaacctaatgaagacggggggtgct

SEQ ID NO: 81 - DNA, 20 bp, Artificial Sequence
  -1584 F1 primer
gctgccatacaatccacctg

SEQ ID NO: 82 - DNA, 21 bp, Artificial Sequence
  -1584 R1 primer
gctcactacaaccttcacctc

SEQ ID NO: 83 - DNA, 18 bp, Artificial Sequence
  100 F2 primer
gtcctgcctggtcctctg

SEQ ID NO: 84 - DNA, 20 bp, Artificial Sequence
  100 R2 primer
cttgccctactcttccttgg

SEQ ID NO: 85 - DNA, 18 bp, Artificial Sequence
  5′1611 primer
gtgggcagagacgaggtg

SEQ ID NO: 86 - DNA, 18 bp, Artificial Sequence
  3′1611 primer
cggagtggttggcgaagg

SEQ ID NO: 87 - DNA, 19 bp, Artificial Sequence
  1758 F1 primer
cttctccgtgtccaccttg

SEQ ID NO: 88 - DNA, 20 bp, Artificial Sequence
  1758 R1 primer
tgtcctttcccaaacccatc

SEQ ID NO: 89 - DNA, 18 bp, Artificial Sequence
  5′ 2573 primer
gtccaggtgaacgcagag

SEQ ID NO: 90 - DNA, 18 bp, Artificial Sequence
  3′ 2573 primer
cggcagagaacaggtcag

SEQ ID NO: 91 - DNA, 21 bp, Artificial Sequence
  5′ 2850 primer
cagagatggagaaggtgagag

SEQ ID NO: 92 - DNA, 19 bp, Artificial Sequence
  3′ 2850 primer
tggaggaggtcaggcttac

SEQ ID NO: 93 - DNA, 21 bp, Artificial Sequence
  4125 F2 primer
actcatcaccaacctgtcatc

SEQ ID NO: 94 - DNA, 23 bp, Artificial Sequence
  4125 R2 primer
ggaactaccacattgctttattg

SEQ ID NO: 95 - DNA, 17 bp, Artificial Sequence
  D&D-F 1 primer
acctctctgggccctca

SEQ ID NO: 96 - DNA, 19 bp, Artificial Sequence
  D&D-R 1 primer
atgccacctcctccttctc

SEQ ID NO: 97 - DNA, 53 bp, Artificial Sequence
  -1584(C)zip15 primer
tcaacgtgccagcgccgtcctgggagctaattttgtattttttgtagagaccg

SEQ ID NO: 98 - DNA, 53 bp, Artificial Sequence
  -1584(G)zip16 primer
gcgaaggaactcgacgtggacgccggctaattttgtattttttgtagagaccc

SEQ ID NO: 99 - DNA, 44 bp, Artificial Sequence
  100(C)Zip27 primer
actacgcaacaccgaacggataccccgctgggctgcacgctacc

SEQ ID NO: 100 - DNA, 44 bp, Artificial Sequence
  100(T)Zip2 primer
cggtcgacgagctgccgcgcaagatcgctgggctgcacgctact

SEQ ID NO: 101 - DNA, 43 bp, Artificial Sequence
  1611(T)zip40 primer
ccccggatagctgacgaggcttacgcccatagcgcgccaggaa

SEQ ID NO: 102 - DNA, 43 bp, Artificial Sequence
  1611(A)zip44 primer
agcagcagtgacaatgccaccgccgcccatagcgcgccaggat

SEQ ID NO: 103 - DNA, 44 bp, Artificial Sequence
  1758(G)Zip101R primer
atctgaaccccgtcctatgtgccgccttctgcccatcacccacc

SEQ ID NO: 104 - DNA, 45 bp, Artificial Sequence
  1758(A)zip109 primer
agcaccccccgtcttcattaggttcccttctgcccatcacccact

SEQ ID NO: 105 - DNA, 43 bp, Artificial Sequence
  2573(G)Zip9 primer
ggctgggtctacagatccccaacttgtcaggtctcgggggggc

SEQ ID NO: 106 - DNA, 43 bp, Artificial Sequence
  2573(C)Zip41 primer
tccggacaggttggggtgcgtttgggtcaggtctcgggggggg

SEQ ID NO: 107 - DNA, 49 bp, Artificial Sequence
  2850(C)zip61 primer
tcggcgtacgagcacccacacccaggaacaggtcagccaccactatgcg

SEQ ID NO: 108 - DNA, 49 bp, Artificial Sequence
  2850(T)Zip31 primer
gagacaaaggtctgcgccagcaccagaacaggtcagccaccactatgca

SEQ ID NO: 109 - DNA, 45 bp, Artificial Sequence
  4125-4133Zip21 primer
ctgaatcctccaaccgggttgtcgagcttctcggtgcccactgga

SEQ ID NO: 110 - DNA, 45 bp, Artificial Sequence
  4125-4133insZip22 primer
ttcggcgctggcgtaaagcttttgggcttctcggtgcccactgtg

SEQ ID NO: 111 - DNA, 46 bp, Artificial Sequence
  6(A)zip106 primer
gtcaggtctaccgcatggagagtgtgccctcagggatgctgctgta

SEQ ID NO: 112 - DNA, 45 bp, Artificial Sequence
  7(C)zip102 primer
cgcacctccaggaccaatcttgttcccctcagggatgctgctgtc

SEQ ID NO: 113 - DNA, 44 bp, Artificial Sequence
  7(C)zip32 primer
tggccacactgtccatttgcgcggtcctcagggatgctgctgtc

SEQ ID NO: 114 - DNA, 44 bp, Artificial Sequence
  6(A)zip19 primer
cgtggcggtgcggagtttccccgaacctcagggatgctgctgta

SEQ ID NO: 115 - DNA, 51 bp, Artificial Sequence
  2D6pc-1460(zip14) primer
ctcggtggtgctgacggtgcaatccccaacatggtgaaaccctatctctac